SEQUENCE LISTING (49 sequences)

SEQ ID NO: 1 - DNA, 504 bp, Artificial Sequence
  Description of Artificial Sequence Synthetic mini-intein derived from the full-length Mycobacterium tuberculosis recA intein sequence
gccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaac

SEQ ID NO: 2 - protein, 168 aa, Artificial Sequence
  Description of Artificial Sequence Synthetic mini-intein derived from the full-length Mycobacterium tuberculosis recA intein sequence
ALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHN

SEQ ID NO: 3 - DNA, 504 bp, Artificial Sequence
  Description of Artificial Sequence Synthetic TOPO modified intein sequence from SEQ ID NO 1
gccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggtccttgtacacaac

SEQ ID NO: 4 - protein, 168 aa, Artificial Sequence
  Description of Artificial Sequence Synthetic TOPO modified intein sequence from SEQ ID NO 2
ALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVLVHN

SEQ ID NO: 5 - DNA, 531 bp, Artificial Sequence
  Description of Artificial Sequence Synthetic GATEWAY modified intein sequence from SEQ ID NO 1
gccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccgacaagtttgtacaaaaaagcaggcagcgcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaac

SEQ ID NO: 6 - protein, 177 aa, Artificial Sequence
  Description of Artificial Sequence Synthetic GATEWAY modified intein sequence from SEQ ID NO 2
ALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPTSLYKKAGSARVQALADALDDKFLHDMLAEELRYSVIREVLPTRRARTFGLEVEELHTLVAEGVVVHN

SEQ ID NO: 7 - DNA, 525 bp, Artificial Sequence
  Description of Artificial Sequence Synthetic TOPO and GATEWAY modified intein sequence from SEQ ID NO 1
gccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccgacaagtttgtacaaaaaagcaggcagcgcgcgcgtgcaggcgctcgcggatgccctggatgacaaattcctgcacgacatgctggcggaagaactccgctattccgtgatccgagaagtgctgccaacgcggcgggcacgaacgttcggcctcgaggaactgcacaccctcgtcgccgaaggggtccttgtacacaac

SEQ ID NO: 8 - protein, 177 aa, Artificial Sequence
  Description of Artificial Sequence Synthetic TOPO and GATEWAY modified intein sequence from SEQ ID NO 2
ALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPTSLYKKAGSARVQALADALDDKFLHDMLAEELRYSVIREVLPTRRARTFGLEVEELHTLVAEGVLVHN

SEQ ID NO: 9 - DNA, 6262 bp, Artificial Sequence
  Description of Artificial Sequence Synthetic pET-GWMIT sequence
caaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccgacaagtttgtacaaaaaagcaggcagcgcgcgcgtgcaggcgctcgcggatgccctggatgacaaattcctgcacgacatgctggcggaagaactccgctattccgtgatccgagaagtgctgccaacgcggcgggcacgaacgttcggcctcgaggaactgcacaccctcgtcgccgaaggggttcttgtacacaacnnnnmnnnnnnnnnnnnnnnnnnnnggtaagcctatccctaaccctctcctcggtctcgattctacgcgtaccggtcatcatcaccatcaccattgagtttgatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatg

SEQ ID NO: 10 - DNA, 6261 bp, Artificial Sequence
  Description of Artificial Sequence Synthetic pET-GWTMIT sequence
caaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccgacaagtttgtacaaaaaagcaggcagcgcgcgcgtgcaggcgctcgcggatgccctggatgacaaattcctgcacgacatgctggcggaagaactccgctattccgtgatccgagaagtgctgccaacgcggcgggcacgaacgttcggcctcgaggaactgcacaccctcgtcgccgaaggggtccttgtacacaacnnnnnnnnnnnnnnnnnnnnnnnnggtaagcctatccctaaccctctcctcggtctcgattctacgcgtaccggtcatcatcaccatcaccattgagtttgatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagggtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatg

SEQ ID NO: 11 - DNA, 6240 bp, Artificial Sequence
  Description of Artificial Sequence Synthetic pET-TMIT sequence
caaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacggacgttcggcctcgaggtcgaggaactgcacaccctcgtcgccgaaggggtccttgtacacaacnnnnnnnnnnnnnnnnnnnnnnnnggtaagcctatccctaaccctctcctcggtctcgattctacgcgtaccggtcatcatcaccatcaccattgagtttgatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatg

SEQ ID NO: 12 - DNA, 27 bp, Artificial Sequence
  Description of Artificial Sequence Synthetic primer
acaagtttgtacaaaaaagcaggcagc

SEQ ID NO: 13 - DNA, 25 bp, Artificial Sequence
  Description of Artificial Sequence Synthetic primer
acaagtttgtacaaaaaagcaggca

SEQ ID NO: 14 - DNA, 24 bp, Artificial Sequence
  Description of Artificial Sequence Synthetic primer
caactttattatacaaagttggca

SEQ ID NO: 15 - DNA, 25 bp, Artificial Sequence
  Description of Artificial Sequence Synthetic primer
gttcaactttattatacaaagttgt

SEQ ID NO: 16 - DNA, 22 bp, Artificial Sequence
  Description of Artificial Sequence Synthetic primer
caacttttctatacaaagttgt

SEQ ID NO: 17 - DNA, 27 bp, Artificial Sequence
  Description of Artificial Sequence Synthetic primer
gttcaacttttctatacaaagttggca

SEQ ID NO: 18 - DNA, 24 bp, Artificial Sequence
  Description of Artificial Sequence Synthetic primer
caacttttctatacaaagttggca

SEQ ID NO: 19 - DNA, 25 bp, Artificial Sequence
  Description of Artificial Sequence Synthetic primer
gttcaacttttctatacaaagttgt

SEQ ID NO: 20 - DNA, 22 bp, Artificial Sequence
  Description of Artificial Sequence Synthetic primer
caacttttgtatacaaagttgt

SEQ ID NO: 21 - DNA, 27 bp, Artificial Sequence
  Description of Artificial Sequence Synthetic primer
gttcaacttttgtatacaaagttggca

SEQ ID NO: 22 - DNA, 24 bp, Artificial Sequence
  Description of Artificial Sequence Synthetic primer
caacttttgtatacaaagttggca

SEQ ID NO: 23 - DNA, 25 bp, Artificial Sequence
  Description of Artificial Sequence Synthetic primer
gttcaacttttgtatacaaagttgt

SEQ ID NO: 24 - DNA, 22 bp, Artificial Sequence
  Description of Artificial Sequence Synthetic primer
caactttttcgtacaaagttgt

SEQ ID NO: 25 - DNA, 27 bp, Artificial Sequence
  Description of Artificial Sequence Synthetic primer
gttcaactttttcgtacaaagttggca

SEQ ID NO: 26 - DNA, 24 bp, Artificial Sequence
  Description of Artificial Sequence Synthetic primer
caactttttcgtacaaagttggca

SEQ ID NO: 27 - DNA, 25 bp, Artificial Sequence
  Description of Artificial Sequence Synthetic primer
gttcaactttttcgtacaaagttgt

SEQ ID NO: 28 - DNA, 27 bp, Artificial Sequence
  Description of Artificial Sequence Synthetic primer
agcctgcttttttatactaacttgagc

SEQ ID NO: 29 - DNA, 27 bp, Artificial Sequence
  Description of Artificial Sequence Synthetic primer
gttcagcttttttatactaagttggca

SEQ ID NO: 30 - DNA, 27 bp, Artificial Sequence
  Description of Artificial Sequence Synthetic primer
agcctgcttttttatactaagttggca

SEQ ID NO: 31 - DNA, 27 bp, Artificial Sequence
  Description of Artificial Sequence Synthetic primer
gttcagcttttttatactaacttgagc

SEQ ID NO: 32 - DNA, 25 bp, Artificial Sequence
  Description of Artificial Sequence Synthetic primer
agcctgcttttttgtacaaacttgt

SEQ ID NO: 33 - DNA, 27 bp, Artificial Sequence
  Description of Artificial Sequence Synthetic primer
gttcagcttttttgtacaaagttggca

SEQ ID NO: 34 - DNA, 27 bp, Artificial Sequence
  Description of Artificial Sequence Synthetic primer
agcctgcttttttgtacaaagttggca

SEQ ID NO: 35 - DNA, 25 bp, Artificial Sequence
  Description of Artificial Sequence Synthetic primer
gttcagcttttttgtacaaacttgt

SEQ ID NO: 36 - DNA, 25 bp, Artificial Sequence
  Description of Artificial Sequence Synthetic primer
acccagctttcttgtacaaagtggt

SEQ ID NO: 37 - DNA, 27 bp, Artificial Sequence
  Description of Artificial Sequence Synthetic primer
gttcagctttcttgtacaaagttggca

SEQ ID NO: 38 - DNA, 27 bp, Artificial Sequence
  Description of Artificial Sequence Synthetic primer
acccagctttcttgtacaaagttggca

SEQ ID NO: 39 - DNA, 25 bp, Artificial Sequence
  Description of Artificial Sequence Synthetic primer
gttcagctttcttgtacaaagtggt

SEQ ID NO: 40 - DNA, 22 bp, Artificial Sequence
  Description of Artificial Sequence Synthetic primer
caactttattatacaaagttgt

SEQ ID NO: 41 - DNA, 27 bp, Artificial Sequence
  Description of Artificial Sequence Synthetic primer
gttcaactttattatacaaagttggca

SEQ ID NO: 42 - DNA, 22 bp, Artificial Sequence
  Description of Artificial Sequence Synthetic primer
caactttttggtacaaagttgt

SEQ ID NO: 43 - DNA, 27 bp, Artificial Sequence
  Description of Artificial Sequence Synthetic primer
gttcaactttttggtacaaagttggca

SEQ ID NO: 44 - DNA, 24 bp, Artificial Sequence
  Description of Artificial Sequence Synthetic primer
caactttttggtacaaagttggca

SEQ ID NO: 45 - DNA, 25 bp, Artificial Sequence
  Description of Artificial Sequence Synthetic primer
gttcaactttttggtacaaagttgt

SEQ ID NO: 46 - DNA, 22 bp, Artificial Sequence
  Description of Artificial Sequence Synthetic primer
caactttttaatacaaagttgt

SEQ ID NO: 47 - DNA, 27 bp, Artificial Sequence
  Description of Artificial Sequence Synthetic primer
gttcaactttttaatacaaagttggca

SEQ ID NO: 48 - DNA, 24 bp, Artificial Sequence
  Description of Artificial Sequence Synthetic primer
caactttttaatacaaagttggca

SEQ ID NO: 49 - DNA, 25 bp, Artificial Sequence
  Description of Artificial Sequence Synthetic primer
gttcaactttttaatacaaagttgt

(19) United States
(12) Patent Application Publication
(10) Pub. No.: US 2009/0098611 A1
(43) Pub. Date: Apr. 16, 2009

(54) Self-cleaving affinity tags and methods of use

(76) Inventors: David W. Wood, Princeton, NJ (US); Judy Hsii, Ashburn, VA (US); Seachol Oak, San Marcos, CA (US); Lydia Contreras, Ithaca, NY (US); Jonathan Chesnut, Carlsbad, CA (US)

Correspondence Address: KATTEN MUCHIN ROSENMAN LLP, 575 MADISON AVENUE, NEW YORK, NY, 10022-2585, US

(21) Appl. No.: 10/591,029

(22) PCT Filed: Feb. 24, 2005
(86) PCT No.: PCT/US05/05763

§ 371 Date: Sep. 20, 2007

Related U.S. Application Data
(60) Provisional application No. 60/548,092, filed on Feb. 27, 2004.

(51) Int. Cl. C12P 19/34; C07H 21/04; C12N 15/64; C12N 9/00; C12N 1/21

(52) U.S. Cl. 435/91.1; 536/23.1; 435/320.1; 435/252.33; 435/183

(57) ABSTRACT

The present invention relates to compositions and methods relating to development of modified inteins comprising one or more topoisomerase recognition sequences and the corresponding topoisomerase proteins and/or one or more recombination sites and the corresponding recombination proteins systems for use in affinity-based protein expression systems.

### BACKGROUND OF THE INVENTION

1. Field of the Invention

The present invention relates to the fields of biotechnology and molecular biology. In particular, the present invention relates to characterization of the self-cleaving activity of modified inteins and their use in facilitating the purification of proteins expressed from vectors employing recombination and/or topoisomerase proteins in their construction. The present invention also relates to cloning nucleic acid fragments using such vectors engineered to contain such modified mutant self-cleaving inteins using recombinational cloning methods such as those employing recombination and/or topoisomerase proteins.

2. Related Art

### Conventional Nucleic Acid Cloning

The cloning of nucleic acid segments currently occurs as a daily routine in many research labs and as a prerequisite step in many genetic analyses. The purpose of these clonings is various, however, two general purposes can be considered: (1) the initial cloning of nucleic acid from large DNA or RNA segments (chromosomes, YACs, PCR fragments, mRNA, etc.), done in a relative handful of known vectors such as pCR2.1, pUC, pGem, pBlueScript, and (2) the subcloning of these nucleic acid segments into specialized vectors for functional analysis. A great deal of time and effort is expended both in the transfer of nucleic acid segments from the initial cloning vectors to the more specialized vectors. This transfer is called subcloning.

The basic methods for cloning have been known for many years and have changed little during that time. A typical cloning protocol is as follows:

(1) digest the nucleic acid of interest with one or two restriction enzymes;

(2) gel purify the nucleic acid segment of interest when known;

(3) prepare the vector by cutting with appropriate restriction enzymes, treating with alkaline phosphatase, gel purify etc., as appropriate;

(4) ligate the nucleic acid segment to the vector, with appropriate controls to eliminate background of uncut and self-ligated vector;

(5) introduce the resulting vector into an E. coli host cell;

(6) pick selected colonies and grow small cultures overnight;

(7) make nucleic acid minipreps; and

(8) analyze the isolated plasmid on agarose gels (often after diagnostic restriction enzyme digestions) or by PCR.

The specialized vectors used for subcloning nucleic acid segments are functionally diverse. These include but are not limited to: vectors for expressing nucleic acid molecules in various organisms; for regulating nucleic acid molecule expression; for providing tags to aid in protein purification or to allow tracking of proteins in cells; for modifying the cloned nucleic acid segment (e.g., generating deletions); for the synthesis of probes (e.g., riboprobes); for the preparation of templates for nucleic acid sequencing; for the identification of protein coding regions; for the fusion of various protein-coding regions; to provide large amounts of the nucleic acid of interest, etc. It is common that a particular investigation will involve subcloning the nucleic acid segment of interest into several different specialized vectors.

As known in the art, simple subclonings can be done in one day (e.g., the nucleic acid segment is not large and the restriction sites are compatible with those of the subcloning vector). However, many other subclonings can take several weeks, especially those involving unknown sequences, long fragments, toxic genes, unsuitable placement of restriction sites, high backgrounds, impure enzymes, etc. One of the most tedious and time consuming type of subcloning involves the sequential addition of several nucleic acid segments to a vector in order to construct a desired clone. One example of this type of cloning is in the construction of gene targeting vectors. Gene targeting vectors typically include two nucleic acid segments, each identical to a portion of the target gene, flanking a selectable marker. In order to construct such a vector, it may be necessary to clone each segment sequentially, i.e., first one gene fragment is inserted into the vector, then the selectable marker and then the second fragment of the target gene. This may require a number of digestion, purification, ligation and isolation steps for each fragment cloned. Subcloning nucleic acid fragments is thus often viewed as a chore to be done as few times as possible.

Several methods for facilitating the cloning of nucleic acid segments have been described, e.g., as in the following references.

Ferguson, J., et al., Gene 16:191 (1981), disclose a family of vectors for subcloning fragments of yeast nucleic acids. The vectors encode kanamycin resistance. Clones of longer yeast nucleic acid segments can be partially digested and ligated into the subcloning vectors. If the original cloning vector conveys resistance to ampicillin, no purification is necessary prior to transformation, since the selection will be for kanamycin.

Hashimoto-Gotoh, T., et al., Gene 41:125 (1986), disclose a subcloning vector with unique cloning sites within a streptomycin sensitivity gene; in a streptomycin-resistant host, only plasmids with inserts or deletions in the dominant sensitivity gene will survive streptomycin selection.

In addition to antibiotic-mediated recombinant selection, recombinant bacteria with cloned inserts may be screened for by means of insertional inactivation of a reporter gene such as lacZα, the structural gene for N-terminus of β-galactosidase (Sambrook et al., Molecular Cloning, A Laboratory Manual, p. 1.85-p. 1.86 Cold Spring Harbor Laboratory Press, Cold Spring Harbor, N.Y.). Synthesis of LacZa from a plasmid molecule compliments the omega fragment of ΔM15lacZ so that functional β-galactosidase is generated. When the desired DNA molecule is cloned into a restriction site within the lacZα gene, this almost always results in an insertional mutation in the gene and results in the loss of β-galactosidase activity. By employing growth medium containing the chromogenic substrate X-gal, the desired recombinants containing the cloned DNA of interest appear as white colonies against a background of blue recombinant colonies containing parental non-recombinant religated vector. Unfortunately though, methods of cloning based upon insertional inactivation of a reporter gene do not select for growth of bacteria with cloned DNA. While it is possible to reduce the incidence of self ligation of parental vectors by employing alkaline phosphatase, this merely reduces but does not eliminate the large of number of colonies that still need to be screened to identify true recombinant clones.

Once a DNA fragment has been successfully cloned, the subsequent purification of the encoded polypeptide from the complex biological mixtures of the expression system often involves a series of complicated steps. Fusion-based affinity separations have helped to provide a simple means of isolating proteins of interest from complex cell extracts by virtue of highly specific interactions between fused affinity proteins and small immobilized ligands (Linder et al. Biotech Bioeng. 1998, 60, 642-647). After protein purification, the protein of interest is cleaved from the fused affinity protein at the fusion joint which has been modified to include a protease recognition sequence. The products produced from such a purification process is a mixture of the polypeptide of interest, the cleaved affinity protein, and a small amount of contaminating protease. Recent advances in recombinant protein purification include the introduction of self-cleaving protein elements called inteins, which eliminate the need for protease addition in protein purifications using fused affinity proteins.

Inteins

Protein splicing is a self-catalyzed process. It is a form of posttranslational processing that involves the excision of an intervening protein sequence from a host protein, accompanied by the concomitant joining of the flanking polypeptides. The intervening protein sequence is known as an intein, while the flanking sequences are called the exteins. In essence, inteins are the protein analogs of self-splicing RNA introns, with the exception that the former is observed in eubacteria, archaea, unicellular eukaryotes and in eukaryotic organelles. There are currently about 150 potential inteins identified (Perler, F. B. Nucleic Acids Res 30, 383-4 (2002); Paulus, H. Front Biosci 8: s1157-65 (2003)), having been found in a variety of important genes such as DNA and RNA polymerases, ATPases, proteases, RecA proteins, and other metabolic enzymes (Pietrokovski, S. Trends Genet 17(8): 465-72 (2001)).

Through sequence analysis, it was discovered that many inteins contain sequences homologous to group I intron-encoded homing endonucleases. These homing endonucleases contain the LAGLIDADG motif (Pietrokovski, S. Protein Sci 3(12): 2340-50 (1994)). It is assumed that endonucleases genes colonized group I introns as they are invasive genetic elements, thereby converting them into mobile genetic elements. The presence of homing endonucleases suggested that inteins are capable of intein homing that is quite similar to intron homing, allowing horizontal transfer of inteins (Liu, X. Q. Annu Rev Genet 34: 61-76 (2000)). With inteins now having been shown in all 3 kingdoms, this further supports the case of them being mobile genetic elements (Perler, F. B. Nucleic Acids Res 30, 383-4 (2002)).

Studies of intein structural analysis suggest that inteins are composed of an endonuclease protein domain and a self-splicing mini-intein domain. The discovery of two separate domains suggest that evolutionarily, the two activities may have evolved independently, and that an endonuclease domain is not necessary for splicing (Derbyshire, V., D. W. Wood, et al. Proc Natl Acad Sci USA 94(21): 11466-71 (1997); (Liu, X. Q. Annu Rev Genet 34: 61-76 (2000)). Experiments further supported that the endonuclease domain was not necessary and could even be deleted to yield a functional splicing mini-intein. One example is the deletion of the entire endonuclease component from the Mycobacterium tuberculosis recA gene, which reduced the 440 amino acid intein to a functional mini-intein of 168 amino acids (Derbyshire, V., D. W. Wood, et al. Proc Natl Acad Sci USA 94(21): 11466-71 (1997)).

The intein itself also contains many conserved elements that are important in its structure and the ability to self-catalyze the splicing process. Of the genetic elements considered to be inteins, most range from 400-500 amino acids. They must be in-frame insertions in a gene with the mature protein product being the same size as the homologs lacking the intein insertion. In addition, the presence of splicing sequence motifs and specific splice junctions are necessary. Ten sequence motifs consisting of blocks A-H, N2 and N4 have been defined ((Pietrokovski, S. Protein Sci 3(12): 2340-50 (1994)); Perler, F. B., G. J. Olsen, et al. Nucleic Acids Res 25(6): 1087-93 (1997)). Blocks C, D, E and H are part of the endonuclease domain and tend to be more conserved than the splicing sequence motifs (Perler, F. B. Nucleic Acids Res 30, 383-4 (2002)). It is thought that these 4 blocks (C, D, E and H) are involved in the recognition, binding and cutting of DNA (Pietrokovski, S. Protein Sci 3(12): 2340-50 (1994)). Motifs A, N2, B and N4 are usually found before the endonuclease domain. F and G are found downstream of the endonuclease domain. The requisite splice junctions for inteins are serine (Ser, S), threonine (Thr, T) or cysteine (Cys, C) at the intein N-terminus and the dipeptide histadine-asparagine (His-Asn, H—N) or histadine-glutamine (His-Gln, H-Q) at the C-terminus. Typically, the first residue after the downstream splice site must be serine, threonine, or cycsteine. Ser, Thr, Cys and Asn are necessary resides in the splicing mechanism, as they act as nucleophiles.

The first residue of most inteins is typically a cysteine, serine or threonine. This residue initiates the splicing reaction by acting as a nucleophile to create an N—S or N—O acyl rearrangement depending on the residue. This forms a linear thioester or ester intermediate. Extein ligation follows with mediation by the highly conserved cysteine, serine or threonine immediately following the intein. Acting as a nucleophile, the sidechain of this residue attacks the ester bond formed in the first step, resulting in transesterification. A branched intermediate is formed. Next, the intein is released when the asparagine at the end of the intein cyclizes to form a succinimide. Lastly, an O—N or S—N acyl rearrangement converts the ester linking the exteins to a peptide bond (Perler, F. B., M. Q. Xu, et al. Curr Opin Chem Biol 1 (3): 292-9 (1997); Liu, X. Q. Annu Rev Genet 34: 61-76 (2000); Paulus 2000; Perler, F. B. and E. Adam Curr Opin Biotechnol 11(4): 377-83 (2000); Xu, M. Q. and T. C. Evans, Jr. Methods 24(3): 257-77 (2001)). Understanding the mechanism of protein splicing gives insights into how the intein can be modified for use in biotechnology. Inteins have now been modified to have the intein cleave instead of splicing ((Liu, X. Q. Annu Rev Genet 34: 61-76 (2000); Xu, M. Q. and T. C. Evans, Jr. Methods 24(3): 257-77 (2001)). For example, when some inteins' N-terminal Cys (C1) is replaced with an Ala (A), N-terminal cleaving and splicing is eliminated with C-terminal cleavage observed (Wood, D. W., W. Wu, et al. Nat Biotechnol 17 (9): 889-92 (1999)). Replacing the Asn (N) in the C-terminal with Ala, C-terminal cleavage and splicing is terminated and N-terminal cleaveage was observed (Chong, S., Y. Shao, et al. J Biol Chem 271(36): 22159-68 (1996); Chong, S., F. B. Mersha, et al. Gene 192(2): 271-81 (1997); Chong, S., G. E. Montello, et al. Nucleic Acids Res 26(22): 5109-15 (1998); Chong, S., K. S. Williams, et al. J Biol Chem 273(17): 10567-77 (1998)). Under other conditions, cleavages at both the N- and C-terminals were observed in place of splicing (Mathys, S., T. C. Evans, et al. Gene 231 (1-2): 1-13 (1999); Southworth, M. W. and F. B. Perler Scientific World Journal 2 (1 Suppl 2): 25-6 (2002)).

It was the ability to block certain splicing steps that allowed the self-cleaving affinity tag to be developed. Wood and co-workers used the Mycobacterium tuberculosis (Mtu) RecA intein for protein purification with C-terminal cleavage of the target protein (Wood, D. W., W. Wu, et al. Nat Biotechnol 17 (9): 889-92 (1999); Wood, D. W., V. Derbyshire, et al. Biotechnol Prog 16(6): 1055-63 (2000)). Chong and colleagues developed a similar single-column purification system using the vacuolar ATPase intein subunit of Saccharomyces cerevisiae (Sce VMA intein) (Chong, S., F. B. Mersha, et al. Gene 192(2): 271-81 (1997); Chong, S., G. E. Montello, et al. Nucleic Acids Res 26(22): 5109-15 (1998)). In each case, the intein was inserted in between the affinity binding protein and the product gene. Cells were induced to overexpress precursor protein followed by conventional purification with affinity binding domains. In both cases, the product protein can then be cleaved from the intein affinity tag while on the column, allowing the recovery of the product protein without addition of protease and additional purification. With the Mtu intein system (Wood and coworkers), the intein cleaving is induced by shifting pH and temperatures. With the Sce intein system (Chong, S., F. B. Mersha, et al. Gene 192(2): 271-81 (1997); Chong, S., G. E. Montello, et al. Nucleic Acids Res 26(22): 5109-15 (1998)), intein cleaving is induced by the addition of high concentrations of thiol-containing compounds (such as dithiothreitol and/or beta-mercaptoethanol). Additional systems have now been reported that use similar strategies to both systems for inducing intein cleaving (Southworth, M. W., Amaya, K., Evans, T. C., Xu, M. Q. & Perler, F. B. (1999) Purification of proteins fused to either the amino or carboxy terminus of the Mycobacterium xenopi gyrase A intein. Biotechniques 27, 110-20). Addition of protease and extra purification steps are thus eliminated in all cases.

Wood and colleagues also experimented and characterized Mtu RecA inteins with the endonuclease domain deleted, creating mini inteins. Furthermore, they were able to create mutated rapid-splicing and cleaving varieties. Characterization showed that the mini-cleaving intein (ΔI-CM) was the most useful for protein purification (Wood, D. W., W. Wu, et al. Nat Biotechnol 17 (9): 889-92 (1999)).

PCT Application No. PCT/US00/33546 (WO 01/42509)(Published U.S. Patent Application No. US20020007051, the entire contents of which are incorporated herein by reference in its entirety) relates, inter alia, to compositions and methods for removing amino acid residues encoded by recombination sites from protein expression products by protein splicing which involves the positioning of nucleic acid sequences which encode intein splice sites on both the 5′ and 3′ end of recombination sites positioned between two coding regions. Thus, when the protein expression product is incubated under suitable conditions, amino acid residues encoded by the recombination sites are excised.

Notwithstanding the usefulness of the prior intein-based expression systems using the mini-cleaving intein (ΔI-CM), there is continuing need in the art to develop other more versatile cloning and expression systems to enhance the efficiency and full range of cloning possibilities. This invention solves these and other long felt needs by providing compositions comprising nucleotide sequences encoding modified intein or a functional derivatives or homologs thereof and methods of utilizing the modified inteins or functional derivatives or homologs thereof in prokaryotic and eukaryotic-based cloning and expression systems.

### SUMMARY OF THE INVENTION

In general, the invention relates to an in vitro and/or in vivo cloning and affinity-fusion based protein expression scheme that can be used with a variety of host cells, including prokaryotic and eukaryotic cells. The invention is based, in part, upon the fact that nucleic acid sequences encoding modified inteins with enhanced, controllable cleavage activity may be combined with one or more topoisomerase recognition sequences and the corresponding topoisomerase proteins and/or one or more recombination sites and the corresponding recombination proteins. One advantage of this combination is to achieve rapid cloning capability coupled with a rapid simple purification method for the expressed protein products.

The invention thus relates, in part, to nucleotide sequences encoding modified inteins or functional derivatives or homologs thereof comprising one or more (e.g., one, two, three, four, five, six, seven eight, etc.) topoisomerase recognition sequences and the corresponding topoisomerase proteins (e.g., a covalently linked topoisomerase) and/or one or more (e.g., one, two, three, four, five, six, seven eight, etc.) recombination sites and the corresponding recombination proteins.

In one embodiment, the nucleotide sequences encoding modified inteins or functional derivatives or homologs thereof may be adjacent to or flank the one or more topoisomerase recognition sequences and the corresponding topoisomerase proteins and/or one or more recombination sites and the corresponding recombination proteins systems. In yet another embodiment, the nucleotide sequences encoding one or more topoisomerase recognition sequences and the corresponding topoisomerase proteins and/or one or more recombination sites and the corresponding recombination proteins may be embedded within nucleotide sequences encoding the modified inteins or functional derivatives or homologs thereof. In yet another embodiment, the nucleotide sequences encoding the one or more topoisomerase recognition sequences and the corresponding topoisomerase proteins and/or one or more recombination sites and the corresponding recombination proteins may be overlapping with the nucleotide sequences encoding modified inteins or functional derivatives or homologs thereof may.

The invention further relates, in part, to nucleotide sequences encoding modified inteins or functional derivatives or homologs thereof comprising one or more topoisomerase recognition sequences and the corresponding topoisomerase proteins and/or one or more recombination sites and the corresponding recombination proteins systems, wherein the nucleotide sequence further encodes a protein of interest that has been modified to contain one or more self-cleaving sequence tags (e.g., affinity tags). In one embodiments, the nucleotide sequences encoding one or more self-cleaving sequence tags (e.g., affinity tags) are embedded within the nucleotide sequences encoding modified inteins or functional derivatives or homologs thereof either alone or in combination with flexible linker sequences of varying length (e.g., about 5-10, 5-15, 5-20, or 5-25 nucleotides, etc.). In another embodiment, the nucleotide sequences encoding one or more self-cleaving sequence tags (e.g., affinity tags) are adjacent to or flanking the nucleotide sequences encoding modified inteins or functional derivatives or homologs thereof either alone or in combination with flexible linker sequences of varying length (e.g., about 5-10, 5-15, 5-20, or 5-25 nucleotides, etc.). In any event, the insertion of the one or more self-cleaving sequence tags (e.g., affinity tags) into the context of the intein or modified intein or functional derivative or homolog thereof described herein is performed at a permissive site for insertion so as to not interfere with the ability of the intein to perform the intein-mediated cleavage reaction.

The invention also relates, in part, to utilization of modified intein nucleotide sequences or functional derivatives or homologs thereof comprising one or more topoisomerase recognition sequences and the corresponding topoisomerase proteins and/or one or more recombination sites and the corresponding recombination proteins systems in prokaryotic and eukaryotic-based cloning and expression systems. The invention relates, in part, to nucleotide sequences encoding modified inteins or functional derivatives or homologs thereof comprising one or more topoisomerase recognition sequences and the corresponding topoisomerase proteins and/or one or more recombination sites and the corresponding recombination proteins systems, wherein the nucleotide sequence further encodes a protein of interest that has been modified to contain one or more self-cleaving sequence tags (e.g., affinity tags).

The invention also relates, in part, to providing vectors comprising nucleotide sequences encoding modified inteins of the invention or a functional derivative or homolog thereof comprising one or more topoisomerase recognition sequences and the corresponding topoisomerase proteins and/or one or more recombination sites and the corresponding recombination proteins systems. In another aspect, the invention also relates, in part, to providing vectors, e.g., recombinant cloning vectors (e.g., donor, entry, destination or expression vectors), comprising a nucleotide sequence comprising the modified intein nucleotide sequences of the invention or a functional derivative or homolog thereof.

In yet another aspect, the invention provides host cells containing such a vector (e.g., donor, entry, destination or expression vectors) or host cells otherwise engineered to contain and/or express the modified intein nucleotide sequences of the invention or a functional derivative or homolog thereof comprising one or more topoisomerase recognition sequences and the corresponding topoisomerase proteins and/or one or more recombination sites and the corresponding recombination proteins systems, as well as methods for making and using such host cells, for example, to produce expression products (e.g., proteins, polypeptides, antigens, antigenic determinants, epitopes, and the like, or fragments thereof).

In yet another aspect, the invention provides methods for constructing such a vector (e.g., donor, entry, destination or expression vectors) capable of expressing the modified intein nucleotide sequences of the invention or a functional derivative or homolog thereof comprising one or more topoisomerase recognition sequences and the corresponding topoisomerase proteins and/or one or more recombination sites and the corresponding recombination proteins systems.

In yet another aspect, the invention provides methods for constructing such a vector (e.g., donor, entry, destination or expression vectors) capable of expressing a modified intein nucleotide sequence of the present invention or a functional derivative or homolog thereof comprising one or more topoisomerase recognition sequences and the corresponding topoisomerase proteins and/or one or more recombination sites and the corresponding recombination proteins systems, wherein a protein of interest encoded by a nucleotide sequence is further modified to contain a nucleotide sequence encoding one or more self-cleaving sequence tags (e.g., affinity tags), wherein the self-cleaving property of the one or more sequence tags is achieved by incorporation of the one or more modified inteins of the present invention or functional derivatives or homologs thereof.

Thus, in one aspect, the nucleotide sequence encoding the protein of interest may be further modified to contain a gene, portion of genes or a nucleotide sequence encoding one or more sequence tags (such as GUS, GST, GFP, His tags, epitope tags and the like) provided by the vectors to allow creation of populations of gene fusions with the desired product molecules cloned in the vector or allows production of a number of peptide, polypeptide or protein fusions encoded by the sequence tags provided by the vector in combination with the desired product sequences cloned in such vector. Such genes, portions of genes or nucleotide sequences encoding one or more sequence tags (e.g., affinity tags) may be used in combination with optionally suppressed stop codons to allow controlled expression of fusion proteins encoded by the sequence of interest being cloned into the vector and the vector supplied gene or nucleotide sequence encoding one or more tag sequences. In a construct, the vector may comprise one or more one or more topoisomerase recognition sequences and the corresponding topoisomerase proteins and/or one or more recombination sites and the corresponding recombination proteins systems, one or more stop codons and a nucleotide sequence encoding one or more tag sequences wherein the tag sequence (e.g., affinity tag) is further modified to comprise at least one modified intein polypeptide or a functional derivative or homolog thereof.

In one embodiment, nucleic acids encoding the tag may be adjacent to, embedded within or overlap with a TOPO® recognition site and/or a GATEWAY® recombination site. Optionally, a stop codon may be incorporated into the nucleotide sequence of the tag (e.g., affinity tag) or in the sequence of the TOPO® recognition site and/or a GATEWAY® recombination site in order to allow controlled addition of the tag nucleotide sequence (e.g., affinity tag) to the gene of interest. In any event, it is intended herein that such a gene, portion of genes or nucleotide sequence encoding the one or more sequence tag(s) (e.g., affinity tags) will not in any way affect the ability of the at least one modified intein or a functional derivative or homolog thereof to exhibit enhanced, controllable cleavage activity following expression of the protein of interest. Cleavage of the at least one one modified intein protein sequence under the appropriate conditions (e.g., temperature and pH) serves to release the sequence tag (e.g., an affinity tag) from the protein of interest.

In yet another aspect, the modified intein polypeptide sequences of the invention or functional derivatives or homologs thereof exhibit controllable cleavage and/or cleavage and splicing activity by varying one or more chemical and/or one or more physical conditions. In certain embodiments, the cleavage and/or cleavage and splicing ability of the modified intein or a functional derivative or homolog thereof may be achieved by varying one or more of pH, temperature, ionic strength and/or oxidative potential. In another embodiment, the cleavage and/or cleavage and splicing ability of the modified intein may be achieved by varying the temperature and/or pH of the intein-mediated cleavage reaction.

Thus, in a first aspect, the invention relates, in part, to intein nucleic acid molecules encoding a modified intein (for example, and not by way of limitation, SEQ ID NOs: 1, 3, 5 or 7, respectively), and the modified intein amino acid sequences encoded by the intein nucleotide sequences (for example, and not by way of limitation, SEQ ID NOs: 2, 4, 6, or 8, respectively).

The intein proteins, fragments, derivatives, and variants thereof are collectively referred to herein as “polypeptides of the invention” or “proteins of the invention.” Nucleic acid molecules encoding the polypeptides or proteins of the invention are collectively referred to as “nucleic acids of the invention.” A polypeptide of the invention exhibits at least one structural and/or functional feature. For example, in the case of the modified intein proteins of the present invention, one structural and/or functional feature is the cleaving activity of the modified intein proteins.

The invention also features nucleic acid molecules which are at least 30%, 35%, 40%, 45%, 50%, 55%, 65%, 75%, 85%, 95%, 98%, or 99% identical to the nucleotide sequences of SEQ ID NOs: 1, 3, 5, or 7, or a complement thereof. In many instances, these nucleic acid molecules will encode a polypeptide or protein which retains at least one activity of a protein encoded by a nucleic acid molecule having a nucleotide sequence shown in SEQ ID NOs: 1, 3, 5, or 7.

The invention also features nucleic acid molecules which include a nucleotide sequence encoding a protein having an amino acid sequence that is at least 40%, 45%, 50%, 55%, 60%, 65%, 75%, 85%, 95%, 98%, or 99% identical to the amino acid sequence of SEQ ID NOs: 2, 4, 6, or 8.

Also within the invention are isolated polypeptides or proteins which are encoded by a nucleic acid molecule having a nucleotide sequence that is at least about 30%, preferably 35%, 40%, 45%, 50%, 55%, 60%, 65%, 75%, 85%, 95%, 98%, or 99% identical to the nucleic acid sequence encoding SEQ ID NOs: 2, 4, 6, or 8, and isolated polypeptides or proteins which are encoded by a nucleic acid molecule having a nucleotide sequence which hybridizes under stringent hybridization conditions to a nucleic acid molecule having the nucleotide sequence of SEQ ID NOs: 1, 3, 5, or 7, or a complement thereof.

The invention also features isolated polypeptides or proteins which are encoded by a nucleic acid molecule having a nucleotide sequence that is at least about 30%, preferably 35%, 40%, 45%, 50%, 55%, 60%, 65%, 75%, 85%, 95%, 98%, or 99% identical to a nucleic acid sequence encoding SEQ ID NOs: 2, 4, 6, or 8, and isolated polypeptides or proteins which are encoded by a nucleic acid molecule having a nucleotide sequence which hybridizes under stringent hybridization conditions to a nucleic acid molecule having the nucleotide sequence of SEQ ID NOs: 1, 3, 5, or 7, or a complement thereof, wherein polypeptides or proteins will often also exhibit at least one structural and/or functional feature of a polypeptide of the invention.

An isolated nucleic acid molecule of the invention that hybridizes under stringent conditions to the sequence of SEQ ID NOs: 1, 3, 5, or 7, or a complement thereof, corresponds to a naturally-occurring nucleic acid molecule.

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues.

An isolated nucleic acid molecule encoding a variant protein can be created by introducing one or more nucleotide substitutions, additions or deletions into the nucleotide sequence of SEQ ID NOs: 1, 3, 5, or 7, such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues.

Accordingly, another aspect of the invention pertains to nucleic acid molecules encoding a polypeptide of the invention that contain changes in amino acid residues that are not essential for activity. Such polypeptides differ in amino acid sequence from SEQ ID NOs: 2, 4, 6, or 8, yet retain biological activity. In one embodiment, the isolated nucleic acid molecule includes a nucleotide sequence encoding a protein that includes an amino acid sequence that is at least about 88%, 90%, 95%, 98%, or 99% identical to the amino acid sequence of SEQ ID NOs: 2, 4, 6, or 8.

Accordingly, another aspect of the invention pertains to nucleic acid molecules encoding a polypeptide of the invention that contain changes in amino acid residues that are essential for activity. Such polypeptides differ in amino acid sequence from SEQ ID NOs: 2, 4, 6, or 8, yet retain biological activity. In one embodiment, the isolated nucleic acid molecule includes a nucleotide sequence encoding a protein that includes an amino acid sequence that is at least about 88%, 90%, 95%, 98%, or 99% identical to the amino acid sequence of SEQ ID NOs: 2, 4, 6, or 8. Thus, for example, and not by way of limitation, it is specifically intended herein that amino acids in the intein polypeptide sequence of SEQ ID NOs: 2, 4, 6, or 8 may be substituted with conservative amino acid substitutions so long as the amino acid substitutions do not affect the ability of the intein polypeptide molecule of SEQ ID NOs: 2, 4, 6, or 8 to confer the phenotype of intein-mediated cleaving. In one embodiment, the initial amino acid of the desired product protein is located in close proximity (e.g., 1, 2, 3, 4, 5, 6, 7, 8, 9, or 10, etc.) to the highly conserved histidine-asparagine dipeptide at the C-terminus of the intein (FIG. 8).

In the present invention, this requirement may be met for example, and not by way of limitation, by the modification of the intein to include the one or more Topo recognition sequences and/or Gateway recombination sequences within the coding sequence of the intein. In another embodiment, the product protein being expressed may also have additional amino acids at the N-terminus added during the cloning reaction for specific applications of the present invention. In this embodiment, additional amino acids would become part of the cleaved product protein. In the various embodiments presented infra, the cloning and expression of proteins of interest employing Topo recognition sequences can be carried out by using any of the available means for adding such Topo recognition sequences including, for example, and not by way of limitation, Directional Topo®, Topo Tools®, and Topo Cloning® reactions available from Invitrogen Corporation (Carlsbad, Calif.) (see, for example, FIGS. 11 and 12 of published U.S. Patent Application No. US20030186233, the entire contents of which are herein incorporated by reference in their entirety, as well as those representative Topo® embodiments presented on Page 14 of Appendix B, infra).

In yet another aspect of the invention, some known homologs of inteins as exemplified in Appendix A, or inteins or homologs thereof that are subsequently found to exist in organisms other than those exemplified infra, are specifically contemplated herein for use in the modified intein compositions and methods of the present invention.

In another aspect, the invention relates to starting vector (e.g., donor, entry, destination or expression vectors) nucleic acid molecules or vector (e.g., donor, entry, destination or expression vectors) product molecules of the invention which comprise a nucleotide sequence encoding at least one modified intein or a functional derivative or homolog thereof.

In certain embodiments, vector (e.g., donor, entry, destination or expression vectors) nucleic acid molecules of the invention which comprise a nucleotide sequence encoding at least one modified intein or a functional derivative or homolog thereof, a considerable number of vector components (e.g., a selectable marker (for example, a kanamycin resistance gene) cassette, an ori cassette, a promoter cassette, a tag sequence cassette, and the like) can be inserted into or used to construct vectors (e.g., donor or expression vectors) of the invention.

In embodiments of the present invention in which vector (e.g., donor, entry, destination or expression vectors) nucleic acid molecules harbor the at least one modified intein nucleotide sequence or a functional derivative or homolog thereof, the vector (e.g., donor or expression vector) nucleic acid molecule harboring the at least one modified intein nucleotide sequence or a functional derivative or homolog thereof will often be propagated in cells resistant to or otherwise capable of withstanding the lethal effects of the ccdB toxin, for example E. coli DB3.1™ cells or equivalent, in the case of ccdB-containing constructs (particularly E. coli LIBRARY EFFICIENCY® DB3.1™ Competent Cells).

In one embodiment of the invention, the vector (e.g., donor, entry, destination or expression vectors) nucleic acid molecules of the invention comprising nucleotide sequences encoding at least one modified intein, or a functional derivative or homolog thereof may also further comprise at least one other open reading frame (ORF) (e.g., one, two, three, four, five, seven, ten, twelve, or fifteen ORFs). Such vector (e.g., donor, entry, destination or expression vectors) molecules may also comprise functional sequences typically found on vectors (e.g., primer sites, transcriptional or translation sites or signals, termination sites (e.g., stop codons which may be optionally suppressed), origins of replication, and the like, and preferably comprises nucleic acid sequences that regulate gene expression including transcriptional regulatory sequences and sequences that function as internal ribosome entry sites (IRES). Preferably, at least one of the vector (e.g., donor, entry, destination or expression vectors) molecules comprise nucleotide sequences that function as a promoter. Such vector (e.g., donor, entry, destination or expression vectors) molecules may also comprise transcription termination sequences, selectable markers, restriction enzyme recognition sites, and the like.

In yet another aspect of the present invention, the vector (e.g., donor, entry, destination or expression vectors) molecules comprising nucleotide sequences encoding at least one one modified intein or a functional derivative or homolog thereof may further comprise recombination sites and the corresponding recombinant proteins for these systems may also be used in accordance with the compositions and methods of the present invention. Preferred recombination proteins and mutant or modified recombination sites for use in the invention include those previously described in U.S. Pat. Nos. 5,888,732, 6,171,861, 6,143,557, 6,270,969 and 6,277,608, and co-pending U.S. application Ser. Nos. 09/438,358 (filed Nov. 12, 1999), 09/517,466 (filed Mar. 2, 2000), 09/695,065 (filed Oct. 25, 2000), and 09/732,914 (filed Dec. 11, 2000), and the MultiSite Gateway® system previously described in co-pending U.S. application Ser. No. 10/640,422 (filed Aug. 14, 2003), the disclosures of all of which are specifically incorporated herein by reference in their entireties, as well as those associated with the Gateway® Cloning Technology available from Invitrogen Corporation (Carlsbad, Calif.).

In yet another aspect of the present invention, the vector (e.g., donor, entry, destination or expression vectors) molecules comprising nucleotide sequences encoding at least one modified intein or a functional derivative or homolog thereof may further comprise topoisomerase recognition sequences and the corresponding topoisomerase proteins for these systems may also be used in accordance with the compositions and methods of the present invention. Preferred topoisomerase recognition sequences and the corresponding topoisomerase proteins for use in the invention include those previously described in co-pending U.S. application Ser. No. 10/640,422 (filed Aug. 14, 2003), the disclosure of which is specifically incorporated herein by reference in its entirety, as well as those associated with the Gateway® Cloning Technology available from Invitrogen Corporation (Carlsbad, Calif.).

Each vector (e.g., donor, entry, destination or expression vectors) nucleic acid molecule comprising nucleotide sequences encoding at least one modified intein or a functional derivative or homolog thereof may comprise, in addition to one or more recombination sites and/or one or more topoisomerase recognition sites and/or one or more topoisomerases, a variety of nucleotide sequences (or combinations thereof) including, but not limited to, nucleotide sequences suitable for use as primer sites (e.g., sequences which a primer such as a sequencing primer or amplification primer may hybridize to initiate nucleic acid synthesis, amplification or sequencing), transcription or translation signals or regulatory sequences such as promoters and/or operators, ribosomal binding sites, topoisomerase recognition sequences (or sites), Kozak sequences, and start codons, transcription and/or translation termination signals such as stop codons (which may be optimally suppressed by one or more suppressor tRNA molecules), tRNAs (e.g., suppressor tRNAs), origins of replication, selectable markers (for example, the kanamycin resistance gene, the ampicillin resistance gene, the chloramphenicol resistance gene, the spectinomycin resistance gene or combinations thereof), and genes or portions of genes which may be used to create protein fusion (e.g., N-terminal or carboxy terminal) such as GST, GUS, GFP, sequence tags, open reading frame (orf) sequences, and any other sequence of interest which may be desired or used in various molecular biology techniques including sequences for use in homologous recombination (e.g., gene targeting).

In certain embodiments of the present invention, the at least one modified intein nucleotide sequences or a functional derivative or homolog thereof may be used in conjunction with a negative selection marker (for example, ccdB) in both conventional and recombinational-based cloning and expression systems.

In certain other embodiments of the present invention, the at least one one modified intein nucleotide sequence or a functional derivative or homolog thereof may be used in conjunction with a positive selection marker in both conventional and recombinational-based cloning and expression systems.

In certain other embodiments of the present invention, the vector (e.g., donor, entry, destination or expression vectors) nucleic acid molecules of the invention which comprise a nucleotide sequence encoding at least one one modified intein, or a functional derivative or homolog thereof, may further contain a considerable number of vector components (e.g., a selectable marker (for example, a kanamycin resistance gene)) cassette, an ori cassette, a promoter cassette, a tag sequence cassette, and the like.

In another aspect, the invention relates to a method of cloning comprising: (a) obtaining at least one nucleic acid molecule of interest to be cloned comprising one or more recombination sites; and (b) transferring all or a portion of said molecule into one or more vectors (e.g., donor or expression vectors) comprising a nucleotide sequence encoding at least one one modified intein or a functional derivative or homolog thereof located between one or more topoisomerase recognition sites and/or one or more bound (e.g., covalently bound) topoisomerases and/or one or more recombination sites. The invention further includes vectors (e.g., donor or expression vectors) prepared by such methods, compositions comprising these vectors, and methods of using these vectors.

In another aspect, the invention relates to a method of cloning comprising: (a) obtaining at least one nucleic acid molecule of interest to be cloned comprising one or more (e.g., 1, 2, 3, 4, 5, 6, 7, 8, etc.) recombination sites and/or one or more (e.g., 1, 2, 3, 4, 5, 6, 7, 8, etc.) topoisomerase recognition sites and/or one or more (e.g., 1, 2, 3, 4, 5, 6, 7, 8, etc.) bound (e.g., covalently bound) topoisomerases; and (b) transferring all or a portion of said molecule into one or more vectors (e.g., donor or expression vectors) comprising a nucleotide sequence encoding at least one (e.g., 1, 2, 3, 4, 5, 6, 7, 8, etc.) modified intein or a functional derivative or homolog thereof located between one or more (e.g., 1, 2, 3, 4, 5, 6, 7, 8, etc.) recombination sites and/or one or more (e.g., 1, 2, 3, 4, 5, 6, 7, 8, etc.) topoisomerase recognition sites and/or one or more (e.g., 1, 2, 3, 4, 5, 6, 7, 8, etc.) bound (e.g., covalently bound) topoisomerases. The invention further includes vectors (e.g., donor or expression vectors) prepared by such methods, compositions comprising these vectors, and methods of using these vectors.

In yet another aspect, the invention relates to a method of cloning comprising: (a) obtaining at least one nucleic acid molecule of interest to be cloned comprising one or more recombination sites and/or one or more topoisomerase recognition sites and/or one or more bound (e.g., covalently bound) topoisomerases; and (b) transferring all or a portion of said molecule into one or more vectors (e.g., donor or expression vectors) comprising a nucleotide sequence encoding at least one modified intein or a functional derivative or homolog thereof located adjacent to one or more recombination sites and/or one or more topoisomerase recognition sites and/or one or more bound (e.g., covalently bound) topoisomerases. The invention further includes vectors (e.g., donor or expression vectors) prepared by such methods, compositions comprising these vectors, and methods of using these vectors.

In another aspect, the invention relates to a method of cloning comprising: (a) obtaining at least one nucleic acid molecule of interest to be cloned comprising one or more recombination sites and/or one or more topoisomerase recognition sites and/or one or more bound (e.g., covalently bound) topoisomerases; and (b) transferring all or a portion of said molecule into one or more vectors (e.g., donor or expression vectors) comprising a nucleotide sequence encoding at least one modified intein or a functional derivative or homolog thereof partially overlapping with one or more recombination sites and/or one or more topoisomerase recognition sites and/or one or more bound (e.g., covalently bound) topoisomerases. The invention further includes vectors (e.g., donor or expression vectors) prepared by such methods, compositions comprising these vectors, and methods of using these vectors.

In yet another aspect of the invention, a method is provided for cloning at least one hybrid nucleic acid molecule comprising: (a) providing at least a first population of nucleic acid molecules wherein all or a portion of such molecules contain at least a first and a second recombination site; (b) providing at least a second population of nucleic acid molecules encoding at least one modified intein or a functional derivative or homolog thereof further comprising a third and a fourth recombination site either embedded within, adjacent to, or overlapping with the nucleotide sequence of the modified intein or a functional derivative or homolog thereof, and wherein either the first or the second recombination site of the first population of nucleic acid molecules is capable of recombining with either the third or the fourth recombination site of the nucleotide sequence encoding at least one modified intein, or a functional derivative or homolog thereof of the second population of nucleic acid molecules; (c) conducting a recombination reaction such that all or a portion of the molecules in the first population is recombined with one or more molecules from the second population to form a third population of hybrid nucleic acid molecules comprising a nucleotide sequence encoding at least one modified intein or a functional derivative or homolog thereof a third and a fourth recombination site are either embedded within, adjacent to, or overlapping with the nucleotide sequence of the modified intein or a functional derivative or homolog thereof; (d) cloning the third population of hybrid nucleic acid molecules; and (e) introducing the cloned hybrid nucleic acid molecules into a suitable host cell.

In yet another aspect of the invention, a method is provided for cloning at least one hybrid nucleic acid molecule comprising: (a) providing a first population of first nucleic acid molecules, wherein the first nucleic acid molecule contains a first and a second recombination site; (b) providing a second population of nucleic acid molecules comprising a vector (e.g., donor, entry, destination or expression vectors) molecule containing a nucleic acid molecule encoding at least one modified intein or a functional derivative or homolog thereof, wherein the modified intein nucleotide sequence or a functional derivative or homolog thereof further comprises a third and a fourth recombination site either embedded within, adjacent to, or overlapping with the nucleotide sequence of the modified intein or a functional derivative or homolog thereof, wherein either the first or the second recombination site of the first nucleic acid molecule is capable of recombining with either the third or the fourth recombination site of the at least one modified intein nucleotide sequence contained within the vector (e.g., donor, entry, destination or expression vectors); (c) conducting a recombination reaction such that all or a portion of the first nucleic acid molecules are recombined with all or a portion of the vector (e.g., donor, entry, destination or expression vectors) molecules comprising a nucleic acid molecule encoding at least one modified intein or a functional derivative or homolog thereof to form a third population of hybrid nucleic acid molecules comprising a nucleotide sequence encoding at least one modified intein or a functional derivative or homolog thereof a third and a fourth recombination site are either embedded within, adjacent to, or overlapping with the nucleotide sequence of the modified intein or a functional derivative or homolog thereof; (d) cloning the third population of hybrid nucleic acid molecules; and (e) introducing the cloned hybrid nucleic acid molecules into a suitable host cell.

Thus, in particular embodiments, nucleic acid molecules of the invention will be vectors (e.g., donor or expression vectors). In additional embodiments, the invention includes host cells that contain nucleic acid molecules of the invention, as well as methods for making and using such host cells, for example, to produce expression products (e.g., proteins, polypeptides, antigens, antigenic determinants, epitopes, and the like, or fragments thereof). In various embodiments, the sequence tags (e.g., affinity tags) are removed via cleavage of the at least one modified intein or a functional derivative or homolog thereof by exposure of the at least one modified intein or a functional derivative or homolog thereof under appropriate conditions (e.g., temperature and/or pH).

The nucleic acid sequences to be joined and/or cloned can be derived from any source, and can be naturally occurring and chemically or recombinantly synthesized nucleic acid molecules such as cDNA, genomic DNA, vectors, oligonucleotides, and the like. Furthermore, the nucleic acid sequences can, but need not, contain one or more functional sequences such as gene regulatory elements, origins of replication, splice sites, polyadenylation sites, open reading frames, which can encode, for example, tag sequences, detectable or selectable markers, cell localization domains, or other peptide or polypeptide, and the like. As such, the invention allows any number of nucleic acid sequences, which can be the same or different, to be linked, including, if desired, in a predetermined order or orientation or both.

For standard recombinant cloning methods using the compositions of the present invention, vector (e.g., donor, entry, destination or expression vectors) molecules produced by methods of the invention may comprise any combination of vector (e.g., donor, entry, destination or expression vectors) molecules (or portions thereof) and can be any size and be in any form (e.g., circular, linear, supercoiled, etc.), depending on the starting nucleic acid molecule or segment, the location of restriction sites on the molecule, and the desired order of combination of the nucleotide molecule or segments.

For recombination cloning methods, vector (e.g., donor, entry, destination or expression vectors) molecules produced by methods of the invention may comprise any combination of vector (e.g., donor, entry, destination or expression vectors) molecules (or portions thereof) and can be any size and be in any form (e.g., circular, linear, supercoiled, etc.), depending on the starting nucleic acid molecule or segment, the location of the recombination sites on the molecule, and the order of recombination of the sites.

Any of the vector (e.g., donor, entry, destination or expression vectors) molecules of the invention may be further manipulated, analyzed or used in any number of standard molecular biology techniques or combinations of such techniques (in vitro or in vivo). These techniques include sequencing, amplification, nucleic acid synthesis, protein or peptide expression (for example, fusion protein expression, antibody expression, hormone expression etc.), protein-protein interactions (2-hybrid or reverse 2-hybrid analysis), homologous recombination or gene targeting, and combinatorial library analysis and manipulation. The invention also relates to cloning the nucleic acid molecules of the invention (e.g., by recombinational methods) into one or more vectors (e.g., donor, entry, destination or expression vectors) or converting the nucleic acid molecules of the invention into a vector (e.g., donor, entry, destination or expression vectors) by the addition of certain functional vector sequences (e.g., origins of replication).

In one aspect, recombination and/or topoisomerase-mediated joining is accomplished in vitro and further manipulation or analysis is performed directly in vitro. Thus, further analysis and manipulation will not be constrained by the ability to introduce the molecules of the invention into a host cell and/or maintained in a host cell. Thus, less time and higher throughput may be accomplished by further manipulating or analyzing the molecules of the invention directly in vitro, although in vitro analysis or manipulation can be done after passage through host cells or can be done directly in vivo (while in the host cells).

Nucleic acid fragments flanked by recombination sites are cloned and subcloned using one or more of the Gateway® systems exemplified in the aforementioned issued U.S. patents and/or pending patent applications by replacing a selectable marker (for example, ccdB) flanked by att sites on the recipient plasmid molecule, sometimes termed the vector (e.g., donor, entry, destination or expression vectors). Desired clones are then selected by transformation of a ccdB-sensitive host strain and positive selection for a marker on the recipient molecule. Similar strategies for negative selection (e.g., use of toxic genes) can of course be used in other organisms such as thymidine kinase (TK) in mammals and insects.

The invention also provides vectors (which may be expression vectors) comprising such isolated nucleic acid molecules. In accordance with the present invention, the vectors are modified to further comprise one or more recombination sites and the corresponding recombination proteins and/or topoisomerase recognition sequences and the corresponding topoisomerase proteins embedded within, adjacent to, and/or overlapping with the at least one modified intein nucleotide sequence or a derivative or homolog thereof, and wherein the nucleotide sequence further encodes a protein of interest that has been modified to contain one or more self-cleaving sequence tags (e.g., affinity tags). Exemplary vectors that may be modified according to this aspect of the invention include, but are not limited to, pcDNAGW-DT(sc), pENTR-DT(sc), pcDNA-DEST41, pENTR/D-TOPO, pENTR/SD/D-TOPO, pcDNA3.2/V5/GWD-TOPO and pcDNA6.2/V5/GWD-TOPO, as well as other exemplary vectors disclosed infra. The invention includes vectors which are derivatives of vectors as described herein, as well as uses of these vectors in various described methods and compositions comprising these vectors.

The invention also provides host cells comprising the isolated nucleic acid molecules or vectors of the invention.

The invention also provides compositions comprising one or more nucleic acid segments and/or nucleic acid molecules described herein. Such compositions may comprise one or a number of other components selected from the group consisting of one or more other nucleic acid molecules (which may comprise nucleic acid sequences encoding one or more sequence tags (e.g, affinity tags), recombination sites, topoisomerase recognition sites, topoisomerases, etc.), one or more nucleotides, one or more polymerases, one or more reverse transcriptases, one or more recombination proteins, one or more topoisomerases, one or more buffers and/or salts, one or more solid supports, one or more polyamines, one or more vectors, one or more restriction enzymes and the like. For example, compositions of the invention include, but are not limited to, mixtures (e.g., reaction mixtures) comprising nucleotide sequences encoding modified inteins or functional derivatives or homologs thereof comprising one or more topoisomerase recognition sequences and the corresponding topoisomerase proteins and/or one or more recombination sites and the corresponding recombination proteins systems. Compositions of the invention further include at least one nucleic acid segment comprising (1) nucleotide sequences encoding modified inteins or functional derivatives or homologs thereof comprising one or more topoisomerase recognition sequences and the corresponding topoisomerase proteins and/or one or more recombination sites and the corresponding recombination proteins and (2) one or more additional components. Examples of such additional components include, but are not limited to, nucleic acid sequences encoding one or more sequence tags (e.g., affinity tags); additional nucleic acid segments, which may or may not comprise one or more topoisomerases or topoisomerase recognition sites, one or more recombination sites and the corresponding recombination proteins; buffers; salts; polyamines (e.g., spermine, spermidine, etc.); water; etc.

The invention also provides kits comprising these isolated nucleic acid molecules of the invention, which may optionally comprise one or more (e.g., one, two, three, four, five, six, etc.) additional components selected from the group consisting of one or more topoisomerases, one or more recombination proteins, one or more vectors (e.g., donor or expression vectors), one or more polypeptides having polymerase activity, and one or more host cells. Additional components for use in a kit of the present invention include one or more components selected from the group consisting of: (a) nucleic acid molecules comprising additional recombination sites; (b) one or more reagents (e.g., enzymes) having ligase activity; (c) one or more reagents (e.g., enzymes) having polymerase activity; (d) one or more reagents (e.g., enzymes) having reverse transcriptase activity; (e) one or more reagents (e.g., enzymes) having restriction endonuclease activity; (f) one or more primers; (g) one or more nucleic acid libraries; (h) one or more supports; (i) one or more buffers; (O) one or more detergents or solutions containing detergents; (k) one or more nucleotides; (l) one or more terminating agents; (m) one or more transfection reagents; (n) one or more host cells; and (o) instructions for using the kit components.

Compositions, methods and kits of the invention may be prepared and carried out using a phage-lambda site-specific recombination system. Further, such compositions, methods and kits may preferably be prepared and carried out using the GATEWAY® Recombinational Cloning System and/or the TOPO® Cloning System and/or the pENTR Directional TOPO® Cloning System, which are available from Invitrogen Corporation (Carlsbad, Calif.). The Gateway® Cloning Technology Instruction Manual (Invitrogen Corp.) describes in more detail the systems and is incorporated herein by reference in its entirety.

Other preferred embodiments of the invention will be apparent to one of ordinary skill in the art in light of what is known in the art, in light of the following drawings and description of the invention, and in light of the claims.

### BRIEF DESCRIPTION OF THE DRAWINGS/FIGURES

FIG. 1 is a schematic representation of a recombinational cloning reaction.

FIG. 2 is a schematic representation of the use of the methods of the present invention to clone two nucleic acid segments by performing an LR recombination reaction.

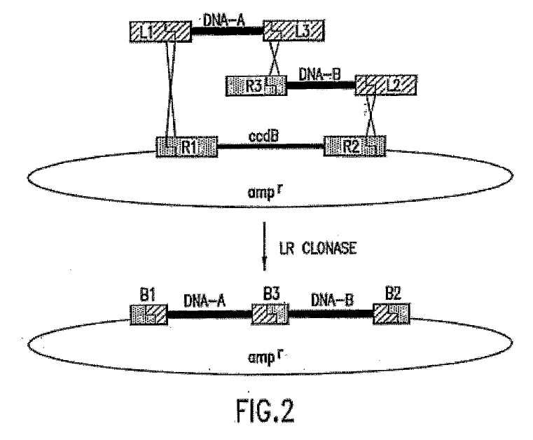

FIG. 3 is a schematic representation of the use of the methods of the present invention to clone two nucleic acid segments by joining the segments using an LR reaction and then inserting the joined fragments into a Destination Vector using a BP recombination reaction.

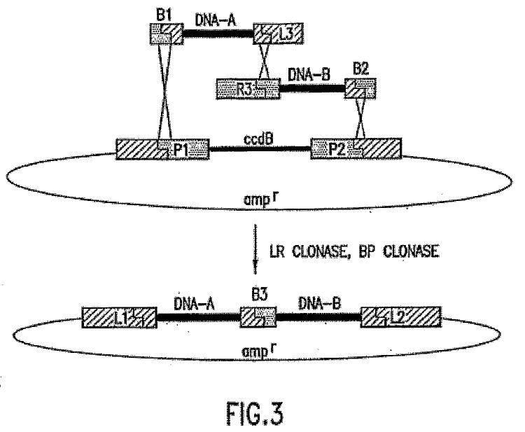

FIG. 4 is a schematic representation of the use of the methods of the present invention to clone two nucleic acid segments by performing a BP reaction followed by an LR reaction.

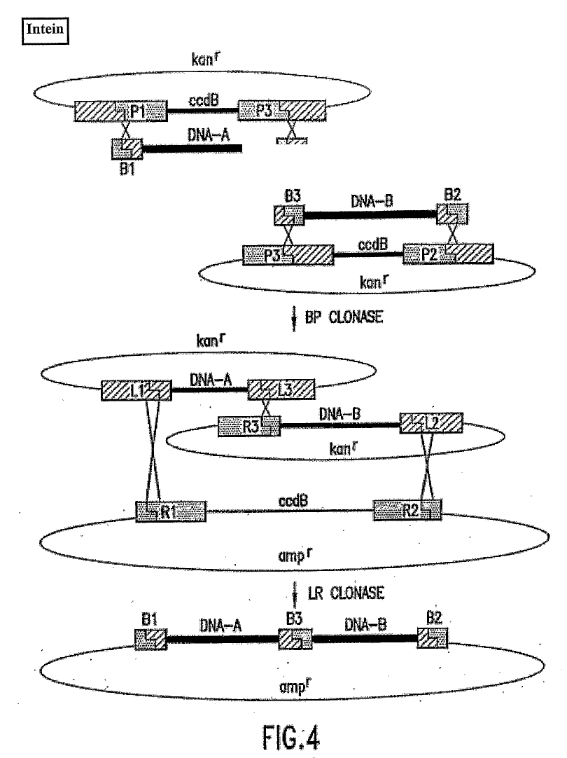

FIG. 5 represents a schematic diagram of vector pET-GWMIT.

FIG. 6 represents a schematic diagram of vector pET-GWTMIT.

FIG. 7 represents a schematic diagram of vector pET-TMIT.

FIG. 8 depicts the conserved C-terminus of a representative intein.

FIG. 9 depicts Topo recognition sequence for representative intein designs.

FIG. 10 depicts cleaving rate studies. Timecourse experiments were performed as follows: Precursor was expressed at 15° C. in ER2566 strain of E. coli for 6 hours uncleaved precursor protein was purified as described in the Materials and Methods Section of Example 1. Samples were taken at time zero, one hour, two hours, four hours, eight hours and overnight (22 hours). Each sample was then analyzed via SDS-Page, and the results are shown for the four inteins.

FIG. 11 depicts cleaving rate studies—optimal pH. Time course experiments are depicted for the Topo+Gateway double intein. The samples and time points were prepared and taken as in FIG. 10. Cleaving of the double intein is shown at 37° C. for the pH levels indicated. It can be seen that the intein cleaves significantly more rapidly at pH 6.5 than at 7.0, and is effectively complete at 8 hours. This cleaving rate is competitive with conventional affinity tag cleaving technologies involving protease addition, and is similar to that achieved with the original ΔI-CM intein.

### DETAILED DESCRIPTION OF THE INVENTION

### Definitions

In the description that follows, a number of terms used in recombinant nucleic acid technology are utilized extensively. In order to provide a clear and more consistent understanding of the specification and claims, including the scope to be given such terms, the definitions to be used are consistent with those used in issued U.S. Pat. Nos. 5,888,732, 6,171,861, 6,143,557, 6,270,969 and 6,277,608, and co-pending U.S. application Ser. Nos. 09/438,358 (filed Nov. 12, 1999), 09/517,466 (filed Mar. 2, 2000), 09/695,065 (filed Oct. 25, 2000), 09/732,914 (filed Dec. 11, 2000), 10/640,422 (filed Aug. 14, 2003)(LTI 2514), and (U.S. Patent Publication 2003/0186233), the disclosures of all of which are specifically incorporated herein by reference in their entireties, as well as those associated with the Gateway® Cloning Technology, available from Invitrogen Corporation (Carlsbad, Calif.).

Gene: As used herein, the term “gene” refers to a nucleic acid which contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. Of course, as would be clearly apparent to one skilled in the art, the transcriptional and translational machinery required for production of the gene product is not included within the definition of a gene. When the gene encodes an untranslated RNA, it includes the promoter and the nucleic acid which encodes the untranslated RNA.

Structural Gene As used herein, the phrase “structural gene” refers to a nucleic acid which is transcribed into messenger RNA that is then translated into a sequence of amino acids characteristic of a specific polypeptide.

Inteins, or Mini-Inteins or Intein Motifs, or Intein Domains: As used herein, by the terms “inteins”, or “mini-inteins” or “intein motifs”, or “intein domains”, or grammatical equivalents herein refer to a protein sequence which, during protein cleaving and/or splicing, is removed or excised from a protein precursor.

Host: As used herein, the term “host” refers to any prokaryotic or eukaryotic organism that is a recipient of a replicable expression vector, cloning vector or any nucleic acid molecule. The nucleic acid molecule may contain, but is not limited to, a structural gene, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Maniatis et al., Molecular Cloning: A Laboratory Manual, Cold Spring Harbor Laboratory, Cold Spring Harbor, N.Y. (1982).

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, and the like.

Promoter: As used herein, a promoter is an example of a transcriptional regulatory sequence, and is specifically a nucleic acid generally described as the 5′-region of a gene located proximal to the start codon or nucleic acid which encodes untranslated RNA. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

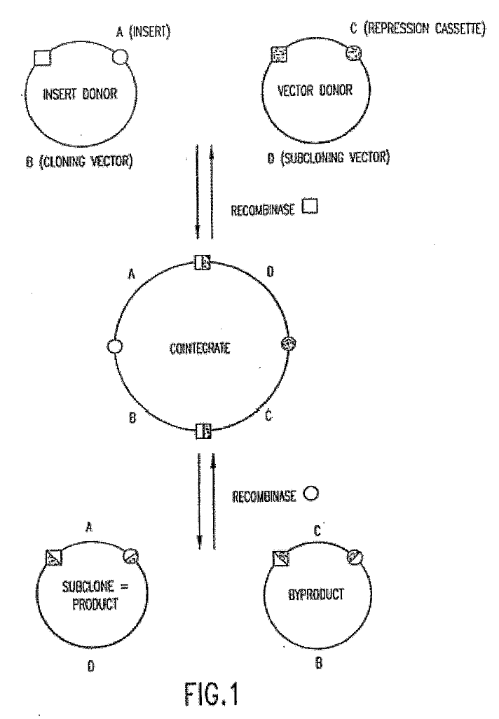

Insert: As used herein, the term “insert” refers to a desired nucleic acid segment that is a part of a larger nucleic acid molecule. In many instances, the insert will be introduced into the larger nucleic acid molecule. For example, the nucleic acid segments labeled intein in FIG. 1, are nucleic acid inserts with respect to the larger nucleic acid molecules shown therein. In most instances, the insert will be flanked by recombination sites (e.g., at least one recombination site at each end). In certain embodiments, however, the insert will only contain a recombination site on one end.

Target Nucleic Acid Molecule: As used herein, the phrase “target nucleic acid molecule” refers to a nucleic acid segment of interest, preferably nucleic acid which is to be acted upon using the compounds and methods of the present invention. Such target nucleic acid molecules preferably contain one or more genes (e.g., two, three, four, five, seven, ten, twelve, fifteen, twenty, thirty, fifty, etc.) or portions of genes.

Naturally-Occurring: As used herein, a “naturally-occurring” nucleic acid molecule refers to an RNA or DNA molecule having a nucleotide sequence that occurs in nature (e.g., encodes a natural protein).

Non-Essential: A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologues of various species may be essential for activity and thus would not be likely targets for alteration. For example, and not by way of limitation, some amino acid substitutions can slow the biological function (e.g., the cleavage reaction and/or splicing reaction) of the inteins of the present invention, but not eliminate it. Thus. specifically contemplated within the definition of non-essential amino acids are those amino acids of the inteins of the present invention that, if mutated, would not interfere with the ability of such slow-splicing or slow-cleaving or otherwise sensitive inteins with these types of mutations. Also specifically contemplated within the definition of non-essential amino acids are those amino acids of the inteins of the present invention that, if mutated, would not interfere with the ability of the inteins with cleaving mutations to be able to perform the cleaveage reaction.

Conservative Amino Acid Substitution: A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Modified Intein Nucleotide Sequence Homologs, or Functional Derivatives Thereof: As used herein, the phrase “modified intein nucleotide sequence homologs, or functional derivatives thereof” is intended to encompass at a minimum those intein nucleotide sequence regions of inteins derived from those inteins referred to or depicted in Appendix A. As defined herein, the phrase “modified intein nucleotide sequence homologs, or functional derivatives thereof” is specifically intended to also encompass the nucleotide sequences of full-length inteins and naturally occurring mini-inteins. Thus, by way of illustration, and not by way of limitation, these intein regions, domains, full length inteins and/or naturally occurring mini-inteins depicted in Appendix A correspond to intein nucleotide sequences encoding inteins domains required for protein function, such as the ability to catalyze a protein splicing reaction and/or exhibit cleavage activity under the appropriate conditions of temperature and pH.

Modified Intein Polypeptide Homologs or Functional Derivatives Thereof: As used herein, the phrases “modified intein polypeptide homologs or functional derivatives thereof” are intended to encompass at a minimum those intein nucleotide sequence regions of inteins derived from those inteins referred to or depicted in Appendix A. As defined herein, the phrase “modified intein polypeptide homologs homologs, or functional derivatives thereof” is specifically intended to also encompass the polypeptides of full-length inteins and naturally occurring mini-inteins. Thus, by way of illustration and not by way of limitation, these intein regions, domains full length inteins and/or naturally occurring mini-inteins depicted in Appendix A correspond to intein nucleotide sequences encoding inteins domains required for protein function, such as the ability to catalyze a protein splicing reaction and/or exhibit cleavage activity under the appropriate conditions of temperature and pH.

Insert Donor: As used herein in accordance with recombination-based cloning methods, the phrase “Insert Donor” refers to one of the two parental nucleic acid molecules (e.g., RNA or DNA) of the present invention which carries the Insert (see FIG. 1). The Insert Donor molecule comprises the Insert flanked on both sides with recombination sites. The Insert Donor can be linear or circular. In one embodiment of the invention, the Insert Donor is a circular nucleic acid molecule, optionally supercoiled, and further comprises a cloning vector sequence outside of the recombination signals. When a population of Inserts or population of nucleic acid segments are used to make the Insert Donor, a population of Insert Donors result and may be used in accordance with the invention.

Product: As used herein in accordance with recombination-based cloning methods, the term “Product” refers to one of the desired daughter molecules comprising the A and D sequences which is produced after the second recombination event during the recombinational cloning process (see FIG. 1). The Product contains the nucleic acid which was to be cloned or subcloned. In accordance with the invention, when a population of Insert Donors are used, the resulting population of Product molecules will contain all or a portion of the population of Inserts of the Insert Donors and preferably will contain a representative population of the original molecules of the Insert Donors.

Byproduct: As used herein in accordance with recombination-based cloning methods, the term “Byproduct” refers to a daughter molecule (a new clone produced after the second recombination event during the recombinational cloning process) lacking the segment which is desired to be cloned or subcloned (see FIG. 1).

Cointegrate: As used herein in accordance with recombination-based cloning methods, the term “Cointegrate” refers to at least one recombination intermediate nucleic acid molecule of the present invention that contains both parental (starting) molecules. Cointegrates may be linear or circular (see FIG. 1). RNA and polypeptides may be expressed from cointegrates using an appropriate host cell strain, for example E. coli DB3.1™ cells in the case of ccdB-containing constructs (particularly E. coli LIBRARY EFFICIENCY® DB3.1™ Competent Cells), and selecting for both selection markers found on the cointegrate molecule.

The presence of the ccdB gene allows negative selection of the donor and destination vectors in E. coli following recombination and transformation. The ccdB protein interferes with E. coli DNA gyrase, thereby inhibiting growth of most E. coli strains (e.g., TOP10, DH5α™). When recombination occurs (i.e. between a destination vector and an entry clone or between a donor vector and an attB PCR product), the ccdB gene is replaced by the gene of interest. Cells that take up unreacted vectors carrying the ccdB gene or by-product molecules retaining the ccdB gene will fail to grow. This allows high-efficiency recovery of the desired clones.

Recognition Sequence: As used herein in accordance with recombination-based cloning methods, the phrase “recognition sequence” refers to a particular sequence to which a protein, chemical compound, DNA, or RNA molecule (e.g., restriction endonuclease, a modification methylase, or a recombinase) recognizes and binds. In the present invention, a recognition sequence will usually refer to a recombination site. For example, the recognition sequence for Cre recombinase is loxP which is a 34 base pair sequence comprising two 13 base pair inverted repeats (serving as the recombinase binding sites) flanking an 8 base pair core sequence. (See FIG. 1 of Sauer, B., Current Opinion in Biotechnology 5:521-527 (1994).) Other examples of recognition sequences are the attB, attP, attL, and attR sequences which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis). (See Landy, Current Opinion in Biotechnology 3:699-707 (1993).) Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way. Examples of topoisomerase recognitions sites include, but are not limited to, the sequence 5′-GCAACTT-3′ that is recognized by E. coli topoisomerase III (a type I topoisomerase); the sequence 5′-(C/T)CCTT-3′ which is a topoisomerase recognition site that is bound specifically by most poxyirus topoisomerases, including vaccinia virus DNA topoisomerase I; and others that are known in the art as discussed elsewhere herein.

Recombination Proteins: As used herein in accordance with recombination-based cloning methods, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

Recombination Site: A used herein in accordance with recombination-based cloning methods, the phrase “recombination site” refers to a recognition sequence on a nucleic acid molecule which participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. (See FIG. 1 of Sauer, B., Curr. Opin. Biotech. 5:521-527 (1994).) Other examples of recognition sequences include the attB, attP, attL, and attR sequences described herein, and mutants, fragments, variants and derivatives thereof, which are recognized by the recombination protein λ Int and by the auxiliary proteins integration host factor (IHF), FIS and excisionase (Xis). (See Landy, Curr. Opin. Biotech. 3:699-707 (1993).) Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site which flanked by recombination sites. For all embodiments involving recombination, the first and second recombination sites do not substantially recombine with each other and the third and fourth recombination sites do not substantially recombine with each other. By the phrase “recombination sites do not substantially recombine with each other”, it is intended that less than about 1, 2, 3, 4, or 5% of the recombination reactions occur between the recited recombination sites. By way of example, and not by way of limitation, less than about 1, 2, 3, 4, or 5% of the recombination reactions occur between the first and second sites and between the third and fourth sites. Also, for all embodiments involving recombination, the first and third recombination site is capable of recombining with the second and fourth recombination site, respectively. Thus, by way of illustration, and not by way of limitation, a first nucleic acid molecule used in the invention may comprise at least a first and second recombination site and a second nucleic acid molecule may comprise at least a third and fourth recombination site, wherein the first and second sites do not recombine with each other and the third and fourth sites do not recombine with each other, although the first and third and/or the second and fourth sites may recombine.

Recombinational Cloning: As used herein, the phrase “recombinational cloning” refers to a method, such as that described in U.S. Pat. Nos. 5,888,732 and 6,143,557 (the contents of each of which are fully incorporated herein by reference), whereby segments of nucleic acid molecules or populations of such molecules are exchanged, inserted, replaced, substituted or modified, in vitro or in vivo. Preferably, such cloning method is an in vitro method.

Selectable Marker: As used herein, the phrase “selectable marker” refers to a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as (β-galactosidase, green fluorescent protein (GFP), yellow flourescent protein (YFP), red fluorescent protein (RFP), cyan fluorescent protein (CFP), and cell surface proteins); (5) nucleic acid segments that bind products which are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence which can be otherwise non-functional (e.g., for PCR amplification of subpopulations of molecules); (10) nucleic acid segments, which when absent, directly or indirectly confer resistance or sensitivity to particular compounds; and/or (11) nucleic acid segments that encode products which either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

Selection Scheme: As used herein in accordance with recombination-based cloning methods, the phrase “selection scheme” refers to any method which allows selection, enrichment, or identification of a desired nucleic acid molecules or host cells contacting them (in particular Product or Product(s) from a mixture containing an Entry Clone or Vector, a Destination Vector, a Donor Vector, an Expression Clone or Vector, any intermediates (e.g., a Cointegrate or a replicon), and/or Byproducts). In one aspect, selection schemes of the invention rely on one or more selectable markers. The selection schemes of one embodiment have at least two components that are either linked or unlinked during recombinational cloning. One component is a selectable marker. The other component controls the expression in vitro or in vivo of the selectable marker, or survival of the cell (or the nucleic acid molecule, e.g., a replicon) harboring the plasmid carrying the selectable marker. Generally, this controlling element will be a repressor or inducer of the selectable marker, but other means for controlling expression or activity of the selectable marker can be used. Whether a repressor or activator is used will depend on whether the marker is for a positive or negative selection, and the exact arrangement of the various nucleic acid segments, as will be readily apparent to those skilled in the art. In some preferred embodiments, the selection scheme results in selection of or enrichment for only one or more desired nucleic acid molecules (such as Products).

Moreover, as used herein, depending upon which particular nucleic acid molecule one wished to select for, it is possible to use a positive and/or negative selection marker. For example, and not by way of limitation, one may select for a nucleic acid molecule by selecting against the presence of nucleic acid molecules containing the ccdB nucleotide sequence that are not the desired nucleic acid molecule (referred to as a “negative selection scheme”) such as for example, the cointegrates and byproducts depicted in FIG. 1. Alternatively, for example, and not by way of limitation, one may select for the presence of nucleic acid molecules not containing the ccdB nucleotide sequence. In this case, for example, the ccdB nucleotide sequence is being used in a positive selection scheme to remove those nucleic acid molecules containing the ccdB nucleotide sequence that are not the desired nucleic acid molecule (referred to as a “positive selection scheme”) such as for example, the cointegrates and byproducts depicted in FIG. 1.

In one embodiment, the selection schemes (which can be carried out in reverse) will take one of three forms, which will be discussed in terms of FIG. 1. The first, exemplified herein with a selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait”.)

Examples of such toxic gene products are well known in the art, and include, but are not limited to, restriction endonucleases (e.g., DpnI, Nla3, etc.); apoptosis-related genes (e.g., ASK1 or members of the bcl-2/ced-9 family); retroviral genes; including those of the human immunodeficiency virus (HIV); defensins such as NP-1; inverted repeats or paired palindromic nucleic acid sequences; bacteriophage lytic genes such as those from ΦX174 or bacteriophage T4; antibiotic sensitivity genes such as rpsL; antimicrobial sensitivity genes such as pheS; plasmid killer genes' eukaryotic transcriptional vector genes that produce a gene product toxic to bacteria, such as GATA-1; genes that kill hosts in the absence of a suppressing function, e.g., kicB, ccdB, ΦX174 E (Liu, Q. et al., Curr. Biol. 8:1300-1309 (1998)); and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention. (See, e.g., U.S. Pat. Nos. 4,960,707 (DpnI and DpnII); 5,000,333, 5,082,784 and 5,192,675 (KpnI); 5,147,800 (NgoAIII and NgoAI); 5,179,015 (FspI and HaeIII): 5,200,333 (HaeII and TaqI); 5,248,605 (HpaII); 5,312,746 (ClaI); 5,231,021 and 5,304,480 (XhoI and XhoII); 5,334,526 (AluI); 5,470,740 (NsiI); 5,534,428 (SstI/SacI); 5,202,248 (NcoI); 5,139,942 (NdeI); and 5,098,839 (PacI). (See also Wilson, G. G., Nucl. Acids Res. 19:2539-2566 (1991); and Lunnen, K. D., et al., Gene 74:25-32 (1988).)

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D.

The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-Specific Recombinase: As used herein, the phrase “site-specific recombinase” refers to a type of recombinase which typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid. (See Sauer, B., Current Opinions in Biotechnology 5:521-527 (1994).) Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

Homologous Recombination: As used herein, the phrase “homologous recombination” refers to the process in which nucleic acid molecules with similar nucleotide sequences associate and exchange nucleotide strands. A nucleotide sequence of a first nucleic acid molecule which is effective for engaging in homologous recombination at a predefined position of a second nucleic acid molecule will therefore have a nucleotide sequence which facilitates the exchange of nucleotide strands between the first nucleic acid molecule and a defined position of the second nucleic acid molecule. Thus, the first nucleic acid will generally have a nucleotide sequence which is sufficiently complementary to a portion of the second nucleic acid molecule to promote nucleotide base pairing.

Homologous recombination requires homologous sequences in the two recombining partner nucleic acids but does not require any specific sequences. As indicated above, site-specific recombination which occurs, for example, at recombination sites such as att sites, is not considered to be “homologous recombination,” as the phrase is used herein.

Vector: As used herein, the terms “vector” refers to a nucleic acid molecule (preferably DNA) that provides a useful biological or biochemical property to an insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment which do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Subcloning Vector: As used herein, the phrase “subcloning vector” refers to a cloning vector comprising a circular or linear nucleic acid molecule which includes, preferably, an appropriate replicon. In the present invention, the subcloning vector (segment D in FIG. 1) can also contain functional and/or regulatory elements that are desired to be incorporated into the final product to act upon or with the cloned nucleic acid insert (segment A in FIG. 1). The subcloning vector can also contain a selectable marker (preferably DNA).

Donor Vector: As used herein, the phrase “Donor Vector” refers to one of the two parental nucleic acid molecules (e.g., RNA or DNA) of the present invention which carries the nucleic acid segments comprising the nucleic acid vector which is to become part of the desired Product. The Donor Vector comprises a subcloning vector D (or it can be called the cloning Destination vector if the Insert Donor does not already contain a cloning vector), and a segment C flanked by recombination sites (see FIG. 1). Segments C and/or D can contain elements that contribute to selection for the desired Product daughter molecule, as described above for selection schemes. Segment B refers to an Entry Vector. The recombination signals can be the same or different, and can be acted upon by the same or different recombinases. In addition, the Donor Vector can be linear or circular.

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

Adapter: As used herein, the term “adapter” refers to an oligonucleotide or nucleic acid fragment or segment (preferably DNA) which comprises one or more recombination sites (or portions of such recombination sites) which in accordance with the invention can be added to a circular or linear Insert Donor molecule as well as other nucleic acid molecules described herein. When using portions of recombination sites, the missing portion may be provided by the Insert Donor molecule. Such adapters may be added at any location within a circular or linear molecule, although the adapters are preferably added at or near one or both termini of a linear molecule. Preferably, adapters are positioned to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule which contains the adapter(s) at the site of cleavage. In other aspects, adapters may be added by homologous recombination, by integration of RNA molecules, and the like. Alternatively, adapters may be ligated directly to one or more and preferably both termini of a linear molecule thereby resulting in linear molecule(s) having adapters at one or both termini. In one aspect of the invention, adapters may be added to a population of linear molecules, (e.g., a cDNA library or genomic DNA which has been cleaved or digested) to form a population of linear molecules containing adapters at one and preferably both termini of all or substantial portion of said population.

Adapter-Primer: As used herein, the phrase “adapter-primer” refers to a primer molecule which comprises one or more recombination sites (or portions of such recombination sites) which in accordance with the invention can be added to a circular or linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers are preferably added at or near one or both termini of a linear molecule. Such adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

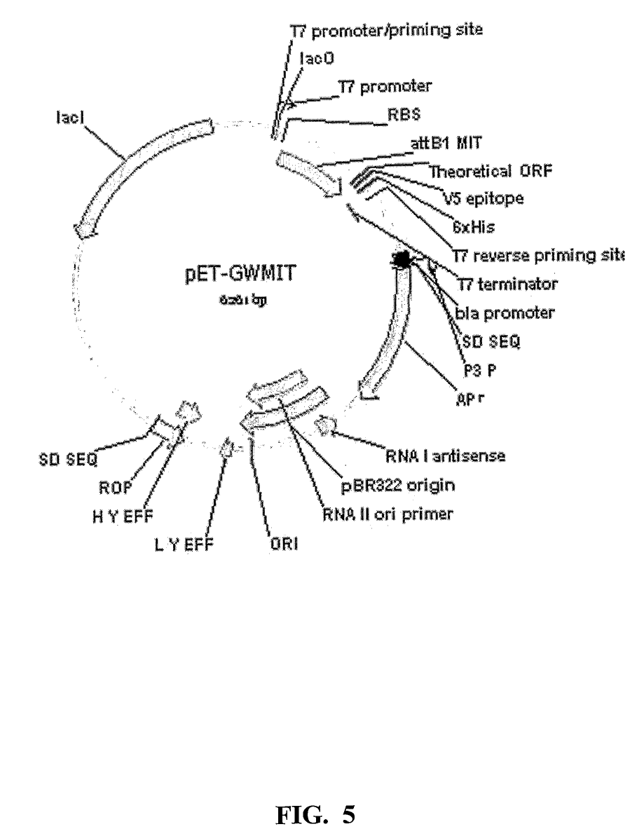

Derivative: As used herein the term “derivative”, when used in reference to a vector, means that the derivative vector contains one or more (e.g., one, two, three, four, five, etc.) nucleic acid segments which share sequence similar to at least one vector represented in one or more of FIGS. 5, 6, and 7. In particular embodiments, a derivative vector (1) may be obtained by alteration of a vector described herein (e.g., a vector represented in FIG. 5, 6, or 7), or (2) may contain one or more elements (e.g., ampicillin resistance marker, attL1 recombination site, TOPO site, etc.) of a vector described herein. Further, as noted above, a derivative vector may contain one or more element that shares sequence similarity (e.g., at least 50%, at least 60%, at least 70%, at least 80%, at least 90%, at least 95%, etc. sequence identity at the nucleotide level) to one or more element of a vector described herein. Derivative vectors may also share at least at least 50%, at least 60%, at least 70%, at least 80%, at least 90%, at least 95%, etc. sequence identity at the nucleotide level to the complete nucleotide sequence of a vector described herein. Thus, derivative vectors include those that have been generated by performing a cloning reaction upon a vector described herein. Derivative vectors also include vectors that have been generated by the insertion of elements of a vector described herein into another vector. Often these derivative vectors will contain at least 50%, at least 60%, at least 70%, at least 80%, at least 90%, at least 95%, etc. of the nucleic acid present in a vector described herein. Derivative vectors also include progeny of any of the vectors referred to above, as well as vectors referred to above which have been subjected to mutagenesis (e.g., random mutagenesis). The invention includes vectors which are derivatives of vectors described herein, as well as uses of these vectors in various described methods and compositions comprising these vectors.

Other terms used in the fields of recombinant nucleic acid technology and molecular and cell biology as used herein will be generally understood by one of ordinary skill in the applicable arts.

Overview

The present invention relates to methods, compositions and kits for the use of self-cleaving affinity tags based upon inteins or functional derivatives or homologs thereof modified to contain one or more recombination sites and/or one or more topoisomerase recognition sequences and use of such modified inteins in GATEWAY® and/or TOPO®-based cloning systems to achieve rapid cloning capability coupled with a rapid and simple purification method for the expressed protein products. Such a unique combination of self-cleaving affinity tags based upon such modified inteins or functional derivatives or homologs thereof with the GATEWAY® and TOPO® cloning methodology permits a significant reduction in the number of recovery steps required during downstream processing of expressed proteins.

The present invention thus relates, in part, to compositions, methods and kits for cloning and/or expression selection systems which employ at least one modified intein nucleotide sequence.

Thus, in one aspect, the invention relates, in part, to utilization of at least one modified intein nucleotide sequence or a functional derivative or homolog thereof in prokaryotic and eukaryotic-based cloning and expression systems. The invention relates, in part, to providing vectors (e.g., recombinant cloning and/or expression vectors), comprising a nucleotide sequence comprising the nucleotide sequences encoding modified inteins of the invention or a functional derivative or homolog thereof. In another aspect, the invention provides methods for constructing such a donor or expression vector expressing the modified intein nucleotide sequences of the invention or a functional derivative or homolog thereof. In another aspect, the invention provides host cells containing such a donor or expression vector engineered to contain and/or express the modified intein nucleotide sequences of the invention or a functional derivative or homolog thereof.

A detailed description is provided herein below of the compositions of the invention, including, non-limiting, representative examples of nucleotide sequences of modified inteins and non-limiting, representative examples of polypeptide sequences of modified inteins, substantially identical variants thereof, homologs thereof, recombination sites and recombinases for use in the compositions of the invention, topoisomerase recognition sites and topoisomerases for use in the compositions of the invention, vectors for use in the compositions and methods of the invention, host cells and cell lines for propagating such modified intein-containing vectors, and methods of using the compositions of the invention, including an overview of cloning strategies or approaches, negative selection vectors based upon the compositions of the invention, positive selection vectors based upon the compositions of the invention, methods of cloning using the compositions of the invention, additional applications for the compositions and methods of the invention, as well as kits for use with the compositions and methods of the invention,

What follows, by way of illustration, and not by way of limitation, is a brief description of the characterization of the modified intein nucleotide sequences of the present invention, and their role in the protein splicing and/or cleavage reaction under suitable conditions sufficient to achieve cleavage of the modified intein protein sequences from the expressed protein of interest.

Those of skill in the art will be able to use the specific teachings and methods provided herein to generate, without undue experimentation, similar vector constructs engineered to contain modified inteins, functional derivatives or homologs thereof, as well as cell lines capable of propagating such vector constructs. Moreover, those of skill in the art will be also able to use the specific teachings and methods provided herein to generate, without undue experimentation, similar vector constructs engineered to contain modified intein nucleotide sequences derived from, for example, one or more of the representative, non-limiting examples of the 140 inteins described in Appendix A infra, as well as cell lines capable of propagating such vector constructs.

By way of background, the following sections provide a brief description of inteins in general, intein engineering for self-cleaving affinity tags, and the behavioral characteristics of the ΔI-CM intein.

Inteins

In 1990, a study of the VMA1 gene from Saccharomyces cerevisiae led to the discovery of a previously unobserved protein behavior (Hirata, R. et al. J Biol Chem 265, 6726-33 (1990)). An analysis of this gene indicated that the expressed VMA enzyme is interrupted by an unusual protein sequence embedded within it. This inner protein has the ability to remove itself and ligate the flanking segments (exteins) to form two separate product proteins. This activity is now know as “protein splicing”, and has been discovered in over 140 host proteins in all three kingdoms (Perler, F. B. Nucleic Acids Res 30, 383-4 (2002)). The internal splicing elements are referred to as inteins (INTervening protEIN sequences), while the outer, spliced proteins are referred to as exteins (Perler, F. B., et al. Nucleic Acids Res 22, 1125-7 (1994); Belfort, M. et al. J Bacteriol 177, 3897-903 (1995)).

Many intein genes are mobile, and can copy themselves into new host genes through a process called “homing” (Gimble, F. S. et al. Nature 357, 301-6 (1992); Doolittle, R. F. et al. Sci Am 269, 50-6 (1993); Belfort, M. et al. J Biol Chem 270, 30237-40 (1995)). An important consequence of intein mobility is a requirement for robust splicing activity in foreign contexts so as to avoid permanently inactivating any newly colonized host protein. This aspect of intein evolution maintains a strong selection for inteins which are active in effectively any protein context. The benefit of this characteristic is that many inteins retain their activity when artificially moved to foreign contexts via conventional cloning. Not only have many inteins been shown to splice efficiently in non-native host cells (Davis, E. O., et al. Cell 71, 201-10 (1992); Perler, F. B., et al. Proc Natl Acad Sci USA 89, 5577-81 (1992); Gu, H. H. et al. J Biol Chem 268, 7372-81 (1993); Liu, X. Q. et al. Febs Lett 408, 311-4 (1997)), but inteins can often also splice out of non-native host proteins (Davis, E. O., et al. Cell 71, 201-10 (1992); Cooper, A. A. et al. Embo J 12, 2575-83 (1993)). Indeed, this generality has been confirmed in the previous work of Wood et al. (Derbyshire, V. et al. Proc Natl Acad Sci USA 94, 11466-71 (1997); Wu, W. et al. Nucleic Acids Res 30, 4864-71 (2002)). Retention of activity in foreign contexts is one major aspect of inteins that makes them attractive for applications in biotechnology.

Most inteins are now known to consist of two large domains. One domain (the splicing domain) is required for protein splicing and cleaving, while the other (the endonuclease domain) is involved with intein mobility. Intein mobility is thought to have arisen when an endonuclease gene homed into an existing self-splicing protein gene to form the observed two-domain bifunctional protein. Recently solved intein structures support this model, indicating two distinct structural domains separated by non-conserved spacer regions of variable length (Duan, X. et al. Cell 89, 555-64 (1997); Ichiyanagi, K. et al. J Mol Biol 300, 889-901 (2000)). Several small inteins have also been identified which lack endonuclease motifs (now referred to as naturally occurring mini-inteins), and in Wood et al.s previous work a functional artificial mini-intein was generated through deletion of the endonuclease domain from a full-length intein (Derbyshire, V. et al. Proc Natl Acad Sci USA 94, 11466-71 (1997); Chong, S. et al. J Biol Chem 272, 15587-90 (1997)).

Highly conserved residues at the intein-extein junctions participate directly in the bond rearrangements that take place during splicing (Chong, S. et al. J Biol Chem 271, 22159-68 (1996); Shao, Y. et al. Chem Biol 4, 187-94 (1997); Paulus, H. Annual Rev Biochem 69, 447-96 (2000); Perler, F. B. et al. Curr Opin Chem Biol 1, 292-9 (1997); Shingledecker, K. et al. Arch Biochem Biophys 375, 138-44 (2000)). The splicing reaction itself is now well understood, and junction residue mutations have been determined that yield various isolated cleaving activities (Chong, S. et al. J Biol Chem 271, 22159-68 (1996); Chong, S. et al. J Biol Chem 273, 10567-77 (1998); Xu, M. Q. et al. Embo J 15, 5146-53 (1996)). An important observation for the present invention is that the reaction step which effectively cleaves the C-terminal intein-extein peptide bond can take place in the absence of splicing, allowing an isolated cleaving event to effectively release the C-extein from the precursor protein (Wood, D. W. et al. Nat Biotechnol 17, 889-92 (1999)). This C-terminal cleavage reaction allows the generation of self-cleaving sequence tags (e.g., affinity tags) and their use in related technologies. As used herein, related technologies is any affinity technology that involves the use of an affinity tag. This would include various methods for purifying proteins with different resins, expression systems and physical configurations. The modified inteins of the present invention may also be used in conjunction with the ability to generate proteins that have amino acids other than methionine at their N-terminus. The C-terminal cleaving reaction makes this possible and the generation of peptides with cysteine at the N-terminus is especially useful for a variety of chemical ligation methods (see Muir, T. W., Sondhi, D. & Cole, P. A. (1998) Expressed protein ligation: a general method for protein engineering. Proc Natl Acad Sci USA 95, 6705-10) or see (Evans, T. C., Jr. & Xu, M. Q. (1999) Intein-mediated protein ligation: harnessing nature's escape artists. Biopolymers 51, 333-42). Thus, the Topo recognition sequence and/or Gateway recombination site modified inteins of the present invention can be used in any technology that currently employs C-terminally cleaving inteins (for example, and not by way of limitation, self-cleaving affinity tags, protein ligation, etc. All inteins are believed to follow the same basic mechanism for protein cleaving, and can be similarly modified for isolated cleaving at one or both ends.

Intein Engineering for Self-Cleaving Affinity Tags

In Wood et al.'s prior work, the Mycobacterium tuberculosis recA intein served as the starting point. The development of Mycobacterium tuberculosis recA intein for the generation of practical self-cleaving sequence tags consisted of three major modifications. In the first modification, the endonuclease domain of the intein was deleted, resulting in a 60% decrease in the intein's molecular weight. The resulting intein is referred to as ΔI (the mini-intein). In the second modification, the initial amino acid of the intein was mutated from cysteine to alanine. This modification effectively prevents the initial acyl shift of the splicing reaction, forcing a C-terminal cleaving reaction. Once these two modifications were made, Wood et al. found that the resulting intein had lost most of its activity, and would require additional modifications in order to be practical for protein purification. To accomplish this, Wood et al. constructed a thymidylate synthase reporter system, where the cleaving intein is genetically fused to a thymidylate synthase gene. In the resulting expressed fusion protein, the thymidylate synthase enzyme is inactive, but is re-activated by the cleaving of the intein. In Escherichia coli cells deficient in thymidylate synthase, efficient cleaving by the intein is required for growth under certain conditions. This reporter system allowed Wood et al. to isolate a rapidly cleaving and controllable mutant of the ΔI intein. This intein is referred to as ΔI-CM (CM=Cleaving Mutant).

The modifications performed on the ΔI-CM intein can, in principle, be performed by one of skill in the art on any intein derived from, for example, one or more of the 140 inteins described in Appendix A infra. The boundaries between the splicing and cleaving domains of a given intein, for example, one or more of the inteins depicted in Appendix A, can be determined by simple phylogenetic analysis, and the endonuclease domain can be deleted through a number of conventional recombinant DNA techniques. Furthermore, naturally occurring mini-inteins have been identified that would not require any modification.

As mentioned above, several modifications of the amino acids at the ends of the intein can be modified to yield isolated cleaving events. Indeed, many modifications often lead to unwanted isolated cleaving in modified inteins. A number of these mutations have been published (Xu, M. Q. et al. Embo J 15, 5146-53 (1996)). The development of the rapidly-cleaving mutant based upon ΔI-CM involved the construction of a genetic selection system, which system has been published in Wood, Wu et al. 1999 and Wood, Derbyshire et al. 2000, and is disclosed in PCT application No. PCT/US00/22581 (WO 01/12820), the disclosure of which is specifically incorporated herein by reference in its entirety. It is important to note that several competing genetic selection systems for intein function have now been published (for example, and not by way of limitation, Adam, E. & Perler, F. B. Development of a positive genetic selection system for inhibition of protein splicing using mycobacterial inteins in Escherichia coli DNA gyrase subunit A. J Mol Microbiol Biotechnol 4, 479-87 (2002), and Daugelat, S. & Jacobs, W. R., Jr. The Mycobacterium tuberculosis recA intein can be used in an ORFTRAP to select for open reading frames. Protein Sci 8, 644-53 (1999), and Gangopadhyay, J. P., Jiang, S. Q. & Paulus, H. An in vitro screening system for protein splicing inhibitors based on green fluorescent protein as an indicator. Anal Chem 75, 2456-62 (2003), and Lew, B. M. & Paulus, H. An in vivo screening system against protein splicing useful for the isolation of non-splicing mutants or inhibitors of the RecA intein of Mycobacterium tuberculosis. Gene 282, 169-177 (2002)], and in principle, any of these competing genetic selection systems could be used to isolate a rapidly cleaving intein. In Wood et al.'s prior work, the mutation that promotes rapid cleaving in the ΔI-CM intein is an aspartic acid to glycine mutation at a highly conserved residue of the F-motif. This specific mutation, and the corresponding mutation in other inteins, is also disclosed in Wood, Wu et al. 1999 and Wood, Derbyshire et al. 2000, and is disclosed in PCT application No. PCT/US00/22581 (WO 01/12820).

Behavioral Characteristics of the ΔI-CM Intein

The initial characterization of Wood et al.'s ΔI-CM intein indicated that it is well-expressed, soluble, and highly sensitive to both pH and temperature in its activity. This combination of sensitivity allows this intein to be used as a linker protein in generating self-cleaving affinity tags. Approximately a dozen test proteins have been fused to the ΔI-CM intein in Wood et al.'s laboratory. These test proteins include: bacteriophage T4 thymidylate synthase, recombinant human acidic fibroblast growth factor, the C-terminal domain of the homing endonuclease I-TevI, beta-lactamase, NusA protein, the sigma subunit of E. coli RNA polymerase, the alpha subunit of E. coli RNA polymerase, the CAP subunit of E. coli RNA polymerase, the homing endonuclease I-TevIII, green fluorescent protein, the E. coli RNA chaperone protein Hfq, the organo-phosphohydrolase enzyme, and the maltose binding domain. Wood et al. have used two different affinity tags to purify proteins of interest, including, for example and not by way of limitation, the maltose binding domain and a recently discovered PHB-binding phasin protein. Other potential affinity tags include the chitin binding protein, glutathione S-transferase, His tag, FLAG tag, cellulose binding proteins, among others. In most cases, Wood et al. used the maltose binding protein as the affinity tag, and have observed that high protein solubility and yields associated with this tag are not affected by the intein insertion.

Wood et al. purified a number of product proteins using this system in combination with the maltose binding protein affinity tag. In each case, the precursor protein is expressed at low temperature (12° C. to 25° C.) to minimize premature cleaving, the expressing cells are lysed at 4° C. into a pH 8.5 buffer to stabilize the uncleaved precursor, and the precursor is purified using standard techniques on an amylose affinity resin. Once the contaminant proteins are washed away, the intein is induced to release the product protein by a shift in pH and/or temperature. Depending on the cleaving temperature, the intein can be induced to cleave in flow mode, or batch mode. Remarkably, the ΔI-CM intein is the only one published that is able to cleave rapidly enough to be used in flow mode. This method represents a very simple and generalizable procedure for purifying arbitrary product proteins, and has been recently commercialized by New England Biolabs (Beverly, Mass.).

The present invention combines Invitrogen Corporation's proprietary Gateway® and Topo® cloning systems with intein-mediated protein purification technology. The combination provides both rapid cloning capability as well as a rapid, simple purification method for the expressed product. A system comprising a Topo®-based vector (e.g. an entry vector) and a series of Gateway® vectors with various combinations of promoters, sequence tags (e.g., affinity tags) and one or modified inteins of the present invention allows researchers to rapidly optimize the cloning and purification of various genes and their products. This combination of technology should find applications in high-throughput, ultra high-throughput cloning and characterization of newly discovered DNA sequences, as Well as in gene library cloning and characterization. For smaller applications, this combination will accelerate the cloning and characterization of individual proteins, and the flexibility of the Gateway® and Topo® cloning systems will allow the rapid optimization of protein expression with self-cleaving affinity tags.

The one or more modifications to the inteins described herein are advantageously used to construct modified inteins capable of exhibiting protein cleaving activity in the context of recombinational and topoisomerase-based cloning and expressions systems. The inventors have generated a series of modified inteins and examined their protein cleavage activities under various conditions. Example 1 infra presents the characterization of the various modified inteins and their use in Gateway recombination site-intein and Gateway recombination site-Topo recognition sequence-intein vectors.

### Compositions of the Invention

### Nucleotide Sequences and Polypeptide Sequences of Modified inteins

Table 1 depicts the nucleotide sequence of the Mycobacterium Tuberculosis Mini Cleaving (ΔI-CM) Intein Nucleotide Sequence (SEQ ID NO:1), Topo Recognition Sequence Intein Nucleotide Sequence (SEQ ID NO:3), Gateway Recombination Site Intein Nucleotide Sequence (SEQ ID NO:5), and the Topo Recognition Sequence-Gateway Recombination Site Intein Nucleotide Sequence (SEQ ID NO:7), respectively, as well as the Mycobacterium Tuberculosis Mini-Cleaving (ΔI-CM) Intein polypeptide sequence (SEQ ID NO:2), Topo Recognition Sequence Intein polypeptide sequence (SEQ ID NO:4), Gateway Recombination Site Intein polypeptide sequence (SEQ ID NO:6), and the Topo Recognition Sequence-Gateway Recombination Site Intein polypeptide sequence (SEQ ID NO:8), respectively. The modified intein nucleotide sequences and amino acid sequences depicted herein in Table 1 are merely illustrative, non-limiting, examples of the compositions of the present invention. The invention specifically contemplates the generation of any intein that has been mutated by genetic selection for rapid and controllable cleaving and modified to contain one or more Topo recognition sequences and/or one or more Gateway recombination sites.

TABLE 1

Mycobacterium Tuberculosis Mini Cleaving (ΔI-CM)
Intein Nucleotide Sequence

(SEQ ID NO: 1)

GCC CTC GCA GAG GGC ACT CGG ATC TTC GAT CCG GTC
ACC GGT ACA ACG CAT CGC ATC GAG GAT GTT GTC GgT
GGG CGC AAG CCT ATT CAT GTC GTG GCT GCT GCC AAG
GAC GGA ACG CTG CAT GCG CGG CCC GTG GTG TCC TGG
TTC GAC CAG GGA ACG CGG GAT GTG ATC GGG TTG CGG
ATC GCC GGT GGC GCC ATC cTG TGG GCG ACA CCC GAT
CAC AAG GTG CTG ACA GAG TAC GGC TGG CGT GCC GCC
GGG GAA CTC CGC AAG GGA GAC AGG GTG GCG CAA CCG
CGA CGC TTC GAT GGA TTC GGT GAC AGT GCG CCG ATT
CCG GCG CGC GTG CAG GCG CTC GCG GAT GCC CTG GAT
GAC AAA TTC CTG CAC GAC ATG CTG GCG GAA GAA CTC
CGC TAT TCC GTG ATC CGA GAA GTG CTG CCA ACG CGG
CGG GCA CGA ACG TTC GgC CTC GAG GTC GAG GAA CTG
CAC ACC CTC GTC GCC GAA GGG GTT GTT GTA CAC AAC
Mycobacterium Tuberculosis Mini Cleaving (ΔI-CM)
Intein Polypeptide Sequence

(SEQ ID NO: 2)

A L A E G T R I F D P V T G T T H R I E D V V G G
R K P I H V V A A A K D G T L H A R P V V S W F D
Q G T R D V I G L R I A G G A I L W A T P D H K V
L T E Y G W R A A G E L R K G D R V A Q P R R F D
G F G D S A P I P A R V Q A L A D A L D D K F L H
D M L A E E L R Y S V I R E V L P T R R A R T F G
L E V E E L H T L V A E G V V V H N
Topo Recognition Sequence Intein Nucleotide
Sequence

(SEQ ID NO: 3)

GCC CTC GCA GAG GGC ACT CGG ATC TTC GAT CCG GTC
ACC GGT ACA ACG CAT CGC ATC GAG GAT GTT GTC GgT
GGG CGC AAG CCT ATT CAT GTC GTG GCT GCT GCC AAG
GAC GGA ACG CTG CAT GCG CGG CCC GTG GTG TCC TGG
TTC GAC CAG GGA ACG CGG GAT GTG ATC GGG TTG CGG
ATC GCC GGT GGC GCC ATC cTG TGG GCG ACA CCC GAT
CAC AAG GTG CTG ACA GAG TAC GGC TGG CGT GCC GCC
GGG GAA CTC CGC AAG GGA GAC AGG GTG GCG CAA CCG
CGA CGC TTC GAT GGA TTC GGT GAC AGT GCG CCG ATT
CCG GCG CGC GTG CAG GCG CTC GCG GAT GCC CTG GAT
GAC AAA TTC CTG CAC GAC ATG CTG GCG GAA GAA CTC
CGC TAT TCC GTG ATC CGA GAA GTG CTG CCA ACG CGG
CGG GCA CGA ACG TTC GgC CTC GAG GTC GAG GAA CTG
CAC ACC CTC GTC GCC GAA GGG GTc cTT GTA CAC AAC
Topo Recognition Sequence Intein Polypeptide
Sequence

(SEQ ID NO: 4)

A L A E G T R I F D P V T G T T H R I E D V V G G
R K P I H V V A A A K D G T L H A R P V V S W F D
Q G T R D V I G L R I A G G A I L W A T P D H K V
L T E Y G W R A A G E L R K G D R V A Q P R R F D
G F G D S A P I P A R V Q A L A D A L D D K F L H
D M L A E E L R Y S V I R E V L P T R R A R T F G
L E V E E L H T L V A E G V L V H N
Gateway Recombination Site Intein Nucleotide
Sequence

(SEQ ID NO: 5)

GCC CTC GCA GAG GGC ACT CGG ATC TTC GAT CCG GTC
ACC GGT ACA ACG CAT CGC ATC GAG GAT GTT GTC GgT
GGG CGC AAG CCT ATT CAT GTC GTG GCT GCT GCC AAG
GAC GGA ACG CTG CAT GCG CGG CCC GTG GTG TCC TGG
TTC GAC CAG GGA ACG CGG GAT GTG ATC GGG TTG CGG
ATC GCC GGT GGC GCC ATC cTG TGG GCG ACA CCC GAT
CAC AAG GTG CTG ACA GAG TAC GGC TGG CGT GCC GCC
GGG GAA CTC CGC AAG GGA GAC AGG GTG GCG CAA CCG
CGA CGC TTC GAT GGA TTC GGT GAC AGT GCG CCG ATT
CCG ACA AGT TTG TAC AAA AAA GCA GGC AGC GCG CGC
GTG CAG GCG CTC GCG GAT GCC CTG GAT GAC AAA TTC
CTG CAC GAC ATG CTG GCG GAA GAA CTC CGC TAT TCC
GTG ATC CGA GAA GTG CTG CCA ACG CGG CGG GCA CGA
ACG TTC GgC CTC GAG GTC GAG GAA CTG CAC ACC CTC
GTC GCC GAA GGG GTT GTT GTA CAC AAC
Gateway Recombination Site Intein Polypeptide
Sequence

(SEQ ID NO: 6)

A L A E G T R I F D P V T G T T H R I E D V V G G
R K P I H V V A A A K D G T L H A R P V V S W F D
Q G T R D V I G L R I A G G A I L W A T P D H K V
L T E Y G W R A A G E L R K G D R V A Q P R R F D
G F G D S A P I P T S L Y K K A G S A R V Q A L A
D A L D D K F L H D M L A E E L R Y S V I R E V L
P T R R A R T F G L E V E E L H T L V A E G V V V
H N
Topo Recognition Sequence-Gateway Recombination
Site Intein Nucleotide Sequence

(SEQ ID NO: 7)

GCC CTC GCA GAG GGC ACT CGG ATC TTC GAT CCG GTC
ACC GGT ACA ACG CAT CGC ATC GAG GAT GTT GTC GgT
GGG CGC AAG CCT ATT CAT GTC GTG GCT GCT GCC AAG
GAC GGA ACG CTG CAT GCG CGG CCC GTG GTG TCC TGG
TTC GAC CAG GGA ACG CGG GAT GTG ATC GGG TTG CGG
ATC GCC GGT GGC GCC ATC cTG TGG GCG ACA CCC GAT
CAC AAG GTG CTG ACA GAG TAC GGC TGG CGT GCC GCC
GGG GAA CTC CGC AAG GGA GAC AGG GTG GCG CAA CCG
CGA CGC TTC GAT GGA TTC GGT GAC AGT GCG CCG ATT
CCG ACA AGT TTG TAC AAA AAA GCA GGC AGC GCG CGC
GTG CAG GCG CTC GCG GAT GCC CTG GAT GAC AAA TTC
CTG CAC GAC ATG CTG GCG GAA GAA CTC CGC TAT TCC
GTG ATC CGA GAA GTG CTG CCA ACG CGG CGG GCA CGA
ACG TTC GgC CTC GAG GAA CTG CAC ACC CTC GTC GCC
GAA GGG GTc cTT GTA CAC AAC
Topo Recognition Sequence-Gateway Recombination
Site Intein Polypeptide Sequence

(SEQ ID NO: 8)

A L A E G T R I F D P V T G T T H R I E D V V G G
R K P I H V V A A A K D G T L H A R P V V S W F D
Q G T R D V I G L R I A G G A I L W A T P D H K V
L T E Y G W R A A G E L R K G D R V A Q P R R F D
G F G D S A P I P T S L Y K K A G S A R V Q A L A
D A L D D K F L H D M L A E E L R Y S V I R E V L
P T R R A R T F G L E V E E L H T L V A E G V L V
H N

The invention features nucleic acid molecules which are at least 30%, 35%, 40%, 45%, 50%, 55%, 65%, 75%, 85%, 95%, 98%, or 99% identical to the nucleotide sequence of SEQ ID NOs: 1, 3, 5, 7, or a complement thereof, as well as compositions (e.g., reactions mixtures) which contain such nucleic acid molecules. Table 1 supra provides the nucleotide sequences of Mycobacterium Tuberculosis Mini Cleaving (ΔI-CM) Intein (SEQ ID NO:1), Topo Intein (SEQ IUD NO:3), Gateway Intein (SEQ ID NO:5), and Topo-Gateway Intein (SEQ ID NO:7), as well as the amino acid sequences encoded by Mycobacterium Tuberculosis Mini Cleaving (ΔI-CM) Intein (SEQ ID NO:2), Topo Intein (SEQ ID NO:4), Gateway Intein (SEQ ID NO:6), and Topo-Gateway Intein (SEQ ID NO:8).

What follows is a detailed description of the mutations required in each of the respective modified inteins, including the mutations present within the ΔI-CM intein, which ΔI-CM intein serves as the parent intein molecule for the generation of the modified inteins or functional derivatives or homologs thereof comprising one or more topoisomerase recognition sequences and the corresponding topoisomerase proteins and/or one or more recombination sites and the corresponding recombination proteins of the present invention.

The ΔI-CM intein (SEQ ID NOs: 1 and 2, respectively) is an artificial mini-intein derived from the full-length Mycobacterium tuberculosis recA intein. To generate this mini-intein, the central endonuclease domain of the full-length intein was first deleted from between nucleotide positions 330 and 331 in the nucleotide sequence depicted in SEQ ID NO: 1. As a result of the deletion of the central endonuclease domain, the ΔI-CM intein only comprises the first 110 and the last 58 amino acids of the original 441-amino acids of the Mtu recA intein. Two additional mutations were then generated. The first mutation is a TCG to GCC mutation of the first codon of the intein (position 1 in the nucleotide sequence depicted in SEQ ID NO: 1), which converts the initial cysteine of the intein to alanine (amino acid position 1 of SEQ ID NO: 2). This mutation prevents splicing and forces an isolated C-terminal cleaving reaction. The other mutation is an A to G at nucleotide position 449 in the nucleotide sequence depicted in SEQ ID NO:1 (shown in lowercase and bold), which results in an aspartic acid (D) to glycine (G) mutation at amino acid position 150 in the amino acid sequence depicted in SEQ ID NO:2. This mutation is important for accelerated cleaving, and must be present for the intein to be of any practical use. In addition to these mutations, some other mutations were isolated over the development of the intein (shown in lowercase in SEQ ID NO: 1). In the preparation of the modified inteins or functional derivatives or homologs thereof comprising one or more topoisomerase recognition sequences and the corresponding topoisomerase proteins and/or one or more recombination sites and the corresponding recombination proteins employed in the generation of the self-cleaving affinity tags of the present invention, these mutations have no effect on intein activity and are only included here for accuracy.

The TOPO® modified intein (SEQ ID NOs: 3 and 4, respectively) was generated from the ΔI-CM intein (SEQ ID NO: 1) through site-directed mutagenesis of the C-terminal region of the intein. Specifically, nucleotide positions 492 and 493 were changed from TG to CC in the nucleotide sequence of SEQ ID NO: 3 (shown in lowercase and bold). This mutation has the effect of changing the last five amino acids of the ΔI-CM intein from VVVHN to VLVHN as depicted in SEQ ID NO: 4 (shown in bold). The mutation has change also creates the DNA sequence TCCTT close to the C-terminus of the intein at nucleotide positions 519-522 of SEQ ID NO:3, which allows the TOPO®-based cloning reaction of the present invention to take place in an efficient manner.

The GATEWAY® modified intein (SEQ ID NOs: 5 and 6, respectively) was generated from the ΔI-CM intein (SEQ ID NO: 1) through insertion of the attB1 sequence into the location of the ΔI-CM intein where the endonuclease domain was originally deleted (nucleotide positions 328-352 of SEQ ID NO:5). Specifically, the attB1 DNA sequence [ACA AGT TTG TAC AAA AAA GCA GGC AGC (SEQ ID NO:12)] was inserted between nucleotide positions 327 and 328 in the original ΔI-CM intein depicted in the nucleotide sequence of SEQ ID NO: 1. This insertion location is very permissive and effectively any att sequence disclosed (e.g. one or more of the att sequences disclosed in Table 2) in the present invention can be inserted at this position in any reading frame with very little impact on the function of the modified inteins. The only proviso is that the attB sequence being inserted preferably not contain a stop codon. Also note that formally, one representative attB1 sequence is ACA AGT TTG TAC AAA AAA GCA GGC A (SEQ ID NO: 13), and the GC located at positions 26 and 27 of SEQ ID NO:13 was added so as to restore the reading frame of the intein DNA after the insertion. The resulting amino acid insertion is TSLYKKAGS as depicted in SEQ ID NO: 6 (amino acid positions 109-117 of SEQ ID NO: 6 shown in bold). This GATEWAY® modified intein will allow the movement of the sequence following the attB1 (which will include the C-terminal part of the GATEWAY® modified intein as well as the target protein of interest with its associated sequence tag (e.g., affinity tag) attached to the end of the GATEWAY® modified intein) to alternate plasmids using GATEWAY®-based recombinational cloning.

The TOPO®-GATEWAY® modified intein (SEQ ID NOs: 7 and 8, respectively) combines the modifications for the TOPO® and GATEWAY® nucleotide and amino acid sequences as previously described above. Specifically, nucleotide residues numbered 519 and 520 of SEQ ID NO: 7 were mutated from TG to CC as in the TOPO® mutation (note that these were originally nucleotide residues numbered 492 and 493, but they have now changed due to the increased length of this TOPO®-GATEWAY® modified intein from the upstream attB1 insertion). The attB1 DNA sequence [ACA AGT TTG TAC AAA AAA GCA GGC AGC (SEQ ID NO:13)] was inserted between residues 327 and 328 in the original ΔI-CM intein. The resulting TOPO®-GATEWAY® modified intein combines the ability of the TOPO® intein to allow rapid generation of entry clones using the TOPO®-based cloning systems of the present invention, with the ability to rapidly move the target protein between expression systems using GATEWAY®-based cloning systems of the present invention. It is expected that the cleaving mutation (aspartic acid to glycine) described for the original ΔI-CM intein will also be important for the function of this TOPO®-GATEWAY® modified intein.

The invention also features nucleic acid molecules which include a nucleotide sequence encoding a protein having an amino acid sequence that is at least 40%, 45%, 50%, 55%, 60%, 65%, 75%, 85%, 95%, 98%, or 99% identical to the amino acid sequence of SEQ ID NOs:2, 4, 6, or 8, or a complement thereof, as well as compositions (e.g., reactions mixtures) which contain such nucleic acid molecules. When determining percent identity for any of the inteins of the present invention, it should be borne in mind that identity among non-allelic inteins is quite low, generally ranging from about 15 to about 30%. For example, pairwise amino acid sequence comparisons indicate that the 11 inteins present in identical locations in DNA polymerase or gyrA genes are more similar to their alleles than to any other intein (at least about 60% identity, except for the Mja pol-2 intein, which is only about 40.4% identical to the Tli pol-1 intein). The VMA inteins are 36.6% identical and branch together in phylogenetic trees. The only intein alleles that fail to phylogenetically group together are the dnaB alleles (about 23% identical), possibly because 46 out of 95 residues used in the analysis are absent in the Ppu dnaB mini-intein. As used herein, an “intein allele” refers to inteins that are present in the same location of homologous genes in different organisms. They generally have higher identity than non-allelic inteins (Perler, F. B., Olsen, G. J. & Adam, E. Compilation and analysis of intein sequences. Nucleic Acids Res 25, 1087-93 (1997)). Sequence similarity between nonallelic inteins is extremely low (most can only be aligned across short sequence regions with low significance scores), but all inteins contain five or six common sequence motifs in the protein-splicing domain that form their active site. The three intein structures that have been solved to date are very similar in their protein-splicing domains (Pietrokovski, S. Intein spread and extinction in evolution. Trends Genet 17, 465-72 (2001)). Thus, the percent identity recitations provided herein are meant to refer at least, in part, to inteins as those peptides which contain recognized intein motifs, particularly at the splice junctions. Such recognized intein motifs and methods for identifying inteins based on these motifs have been published (Pietrokovski, S. Conserved sequence features of inteins (protein introns) and their use in identifying new inteins and related proteins. Protein Sci 3, 2340-50 (1994)).

Also within the invention are isolated polypeptides or proteins which are encoded by a nucleic acid molecule having a nucleotide sequence that is at least about 30%, preferably 35%, 40%, 45%, 50%, 55%, 60%, 65%, 75%, 85%, 95%, 98%, or 99% identical to the nucleic acid sequence encoding SEQ ID NOs: 2, 4, 6, or 8, and isolated polypeptides or proteins which are encoded by a nucleic acid molecule having a nucleotide sequence which hybridizes under stringent hybridization conditions to a nucleic acid molecule having the nucleotide sequence of SEQ ID NOs:1, 3, 5, or 7, or a complement thereof.

The invention also features isolated polypeptides or proteins which are encoded by a nucleic acid molecule having a nucleotide sequence that is at least about 30%, preferably 35%, 40%, 45%, 50%, 55%, 60%, 65%, 75%, 85%, 95%, 98%, or 99% identical to a nucleic acid sequence encoding SEQ ID NOs: 2, 4, 6, or 8, isolated polypeptides or proteins which are encoded by a nucleic acid molecule having a nucleotide sequence which hybridizes under stringent hybridization conditions to a nucleic acid molecule having the nucleotide sequence of SEQ ID NOs: 1, 3, 5, or 7, or a complement thereof, wherein polypeptides or proteins also exhibit at least one structural and/or functional feature of a polypeptide of the invention.

Substantially Identical Mycobacterium Tuberculosis Mini Cleaving (ΔI-CM) Intein, Topo Intein, Gateway Intein, and Topo-Gateway Intein Nucleotide Sequences and Mycobacterium Tuberculosis Mini Cleaving (ΔI-CM) Intein, Topo Intein, Gateway Intein, and Topo-Gateway Intein Polypeptide Sequences

Nucleic acid molecules or segments produced by or used in conjunction with the methods of the invention, as well as nucleic acid molecules or segments thereof of the invention, include those molecules or segments specifically described herein as well as those molecules or segments that have substantial sequence identity to those molecules or segments specifically described herein. By a molecule or segment having “substantial sequence identity” to a given molecule or segment is meant that the molecule or segment is at least about 90%, 91%, 92%, 93%, 94%, 95%, 96%, 97%, 98% or 99%, identical to the given (or “reference”) molecule or segment. By a nucleic acid molecule or segment having a nucleotide sequence at least, for example, 65% “identical” to a reference nucleic acid molecule or segment is intended that the nucleotide sequence of the nucleic acid molecule or segment is identical to that of the reference sequence except that the nucleic acid molecule or segment may include up to 35 point mutations per each 100 nucleotides of the reference nucleotide sequence. In other words, to obtain a polynucleotide having a nucleotide sequence at least 65% identical to a reference nucleotide sequence, up to 5% of the nucleotides in the reference sequence may be deleted or substituted with another nucleotide, or a number of nucleotides up to 35% of the total nucleotides in the reference sequence may be inserted into the reference sequence. These mutations of the reference sequence may occur at the 5′ or 3′ terminal positions (or both) of the reference nucleotide sequence, or anywhere between those terminal positions, interspersed either individually among nucleotides in the reference sequence or in one or more contiguous groups within the reference sequence.

As a practical matter, whether any particular nucleic acid molecule or segment is at least about 65%, 70%, 75%, 80%, 85%, 90%, 95%, 96%, 97%, 98% or 99% identical to a given reference molecule or segment can be determined conventionally using known computer programs such as FASTA (Heidelberg, Germany), BLAST (Washington, D.C.) or BESTFIT (Wisconsin Sequence Analysis Package, Version 8 for Unix, Genetics Computer Group, University Research Park, 575 Science Drive, Madison, Wis. 53711), which employs a local homology algorithm (Smith and Waterman, Advances in Applied Mathematics 2: 482-489 (1981)) to find the best segment of homology between two sequences. When using FASTA, BLAST, BESTFIT or any other sequence alignment program to determine whether a particular sequence is, for instance, 65% identical to a reference sequence according to the present invention, the parameters are set such that the percentage of identity is calculated over the full length of the reference nucleotide sequence and that gaps in homology of up to 35% of the total number of nucleotides in the reference sequence are allowed.

Homologs of Mycobacterium Tuberculosis Mini Cleaving (ΔI-CM) Intein, Topo Intein, Gateway Intein, and Topo-Gateway Intein Nucleotide Sequences and Mycobacterium Tuberculosis Mini-Cleaving (ΔI-CM) Intein, Topo Intein, Gateway Intein, and Topo-Gateway Intein Polypeptide Sequences

In yet another aspect of the invention, homologs of Mycobacterium Tuberculosis Mini Cleaving (ΔI-CM) Intein, Topo Intein, Gateway Intein, and Topo-Gateway Intein that currently exist or are subsequently found to exist in organisms other than E. coli are specifically contemplated for use in the compositions and methods of the present invention. Thus, interchangeable homologs of Mycobacterium Tuberculosis Mini Cleaving (ΔI-CM) Intein, Topo Intein, Gateway Intein, and Topo-Gateway Intein include, for example, and not by way of limitation, the inteins comprising that found in the listing in Appendix A, and thus the present invention also specifically contemplates generation of additional modified inteins which are capable of being used in the recombinational and topoisomerase cloning methods and vectors described herein so long as the inteins are capable of performing the protein splicing and/or cleavage reaction so as to precisely remove the intein amino acid sequence from the expressed protein of interest, and use of these modified inteins in the compositions and methods of the present invention.

Additional Possible Inteins for Use in the Mini Cleaving Intein, Topo Intein, Gateway Intein, and Topo-Gateway Intein Cloning and Expression System

In yet another aspect of the invention, in addition to those substantially identical inteins and intein homologs that may be used in the cloning and expression system of the present invention, other inteins may also be employed to create the Gateway Intein and Topo-Gateway Intein vectors. For example, interchangeable inteins include, for example, and not by way of limitation, the inteins comprising those found in The Intein Registry. This registry includes a list of all experimental and theoretical inteins discovered to date and submitted to the registry (http://www.neb.com/inteins/int reg.html). A non-exhaustive, representative listing of inteins discovered to date and which may be used in the cloning and expression system of the present invention may be found in Appendix A, infra.

More particularly, interchangeable inteins may be selected from among the approximately 163 inteins that have been identified to date and that are available from public databases (Perler, Nucleic Acids Res. 22:1125-1127 (1994), Perler, Nucleic Acids Res. 27:346-347 (1999), Pietrokovski, S., Protein Sci., 7:64-71 (1998) and Dalgaard, et al., J. Comput. Biol., 4:193-214 (1997).

In one embodiment, intein motifs selected from organisms belonging to the Eucarya, Eubacteria and Archea may be modified to contain one or more topoisomerase and/or recombinations sites.

In other embodiments, inteins with alternative splicing mechanisms may be employed in the compositions and methods of the present invention (see Southworth, et al., (2000) EMBO J., 19:5019-26). The GenBank accession numbers for inteins with alternative splicing mechanisms include, but are not limited to, Mja KlbA (GenBank accession number Q58191), and Pfu KIbA (PF.sub.—949263 in UMBI).

In yet other embodiments, inteins from thermophilic organisms may be employed in the compositions and methods of the present invention. Random mutagenesis or directed evolution (i.e. PCR shuffling, etc.) of inteins from these organisms using techniques known to one of skill in the art leads to the isolation of temperature sensitive mutants. Thus, inteins from thermophiles may be employed in the compositions and methods of the present invention and include, but are not limited to, Mth RIR1 (GenBank accession number G69186), Pfu RIR1-1 (AAB36947.1), Psp-GBD Pol (GenBank accession number AAA67132.1), Thy Pol-2 (GenBank accession number CAC18555.1), Pfu IF2 (PF.sub.—1088001 in UMBI), Pho Lon Baa29538.1), Mja r-Gyr (GenBank accession number G64488), Pho RFC (GenBank accession number F71231), Pab RFC-2 (GenBank accession number C75198), Mja RtcB (also referred to as Mja Hyp-2; GenBank accession number Q58095), and Pho VMA (NT01PH 1971 in Tigr).

Conditions and Parameters for Intein-Mediated Cleavage

Previous work in the field of intein research has shown that non-native cleavage can be induced at either end of the intein, but typically the cleavage rate is slow. Chong et al. (J. Biol. Chem. 272:15587-15590 (1997a)); Chong et al. (J. Biol. Chem. 273:10567-10577 (1998a)); Chong et al. (J. Biol. Chem. 271:22159-22168 (1996)); Xu et al. (EMBO J. 15:5146-5153 (1996)) and Chong et al. (Nucl. Acids Res. 26:5109-5115 (1998b)). In these systems, where inteins have been modified for C-terminal cleavage, the reactions can take several days at 4° C., and/or require the addition of a thiol reagent, and can be accompanied by N-terminal cleavage, necessitating an additional purification step. Chong et al. (1998a).

In yet another aspect of the present invention, the modified inteins or functional derivatives or homologs thereof comprising one or more topoisomerase recognition sequences and the corresponding topoisomerase proteins and/or one or more recombination sites and the corresponding recombination proteins of the present invention display rapid, pH-sensitive, isolated C-terminal cleavage which obviates the need for reducing reagents and additional purification steps. Importantly, C-terminal cleavage-based affinity separation times can decrease to several hours at 4° C., or to minutes at higher temperatures, making the temperature-dependent sensitivity of the method of the present invention more attractive for scaleup of TOPO® recognition sequence and/or a GATEWAY® recombination site-modified intein-based protein purifications. The modified inteins or functional derivatives or homologs thereof comprising one or more topoisomerase recognition sequences and the corresponding topoisomerase proteins and/or one or more recombination sites and the corresponding recombination proteins of the present invention exhibit have elevated activities in vivo and in vitro, and therefore form the basis of a pH- and temperature-dependent protein purification system. The dependency of the intein-based cleavage reaction upon pH, temperature, ionic strength and/or oxidative potential is outlined below.

Variation in PH for Intein-Mediated Cleavage

In yet another aspect of the present invention, the specific pH behavior of the modified inteins or functional derivatives or homologs thereof comprising one or more topoisomerase recognition sequences and the corresponding topoisomerase proteins and/or one or more recombination sites and the corresponding recombination proteins of the present invention exhibit an approximate 20- to 40-fold increase in activity between pH 8.5 and 6.0. These pH values are relatively mild, thereby effectively decreasing the potential for damage to the desired product protein due to pH-induced denaturation, and therefore permitting recovery of pure protein with minimal damage and/or contamination. This relatively narrow pH range also decreases the possibility that the binding domain of the one or more sequence tags (e.g., affinity tags) employed will lose affinity during cleavage. A key feature of the modified inteins or functional derivatives or homologs thereof comprising one or more topoisomerase recognition sequences and the corresponding topoisomerase proteins and/or one or more recombination sites and the corresponding recombination proteins of the present invention is their extreme pH sensitivity, which allows purification of intact precursor followed by rapid C-terminal cleavage. Although the conserved His amino acid residue immediately preceding the final Asn amino acid residue of native inteins may be responsible for this effect (Chong et al. (1998a); Duan et al. (Cell 89:555-564 (1997)); and Klabunde et al. (Nature Struct. Biol. 5:31-36 (1998)), with the inteins of the present invention it is possible to use pH-related cleavage sensitivity to accelerate the intein-meadiated cleavage to a useful rate. The modified inteins or functional derivatives or homologs thereof comprising one or more topoisomerase recognition sequences and the corresponding topoisomerase proteins and/or one or more recombination sites and the corresponding recombination proteins of the present invention thus display elevated cleavage activity compared to both the full-length Mycobacterium Tuberculosis intein and its mini-intein parent molecule (Mini Cleaving (ΔI-CM) Intein) making it particularly useful for application in affinity-based separations.

In yet another aspect of the present invention, in the case of affinity-based separations, the expressed fusion protein containing one or more TOPO® recognition sequence and/or a GATEWAY® recombination site-modified intein is then bound to a solid matrix via the affinity group or ligand binding domain. The bound expressed fusion protein can then be washed and subjected to a cleavage reaction or directly subjected to the cleavage reaction. The cleavage reaction can be autocatalytic cleavage, for instance, triggered by a change in one or more physical condition(s) and/or one or more chemical condition(s) e.g., a change in one or more physical condition(s) and/or one or more chemical condition(s), or any combination thereof (e.g., a change in pH, temperature, ionic strength and/or oxidative potential). After the cleavage of the desired product protein from the fusion product, the purified desired product protein is then isolated.

Variation in Temperature for Intein-Mediated Cleavage

In yet another aspect of the present invention, the invention provides modified inteins or functional derivatives or homologs thereof comprising the TOPO® recognition sequence and/or a GATEWAY® recombination sites that display a strong dependence on temperature, thereby allowing uncleaved precursor to be expressed in host cells for purification. Although this method of protein purification implicitly requires that the protein of interest be expressed at low temperatures, virtually total protein precursor (with the modified intein TOPO® recognition sequence and/or a GATEWAY® recombination sites and sequence tag (e.g., affinity tag) tripartite fusion intact) can be generated with almost no premature cleavage taken place.

In the present invention, the time required for the isolated C-terminal cleavage reaction varies depending upon the temperature employed. For example, and not by way of limitation, the isolated C-terminal cleavage reaction can be completed in about 4 hours at 37° C., in about 12 hours at 25° C., in about 30 hours at 20° C. or in about 150 hours at 4° C. In each specific instance, the isolated C-terminal cleavage reaction is about 90-95% complete. It is important to express the tagged precursor protein containing the modified inteins in E. coli at a low temperature to maximize the yield of uncleaved precursor protein. In principle, the expression of the precursor protein can take place at any temperature, but the precursor protein generally will start to cleave prematurely at temperatures at or above 30° C. For most situations, the precursor proteins are expressed at a temperature of 15° C. to 20° C. It is also possible to rapidly produce precursor at higher temperature for shorter amounts of time so as to effectively minimize premature cleaving.

The cells are then lysed into a pH 8.5 buffer to stabilize the uncleaved precursor, and the purification via the sequence tags (e.g., affinity tags) also takes place at pH 8.5. Once all of the contaminant proteins have been removed, the modified intein is then induced to release the product protein by a shift in temperature and/or pH. This cleavage reaction can be accomplished in a batch type mode, where the pH of the column itself is rapidly shifted and the column is sealed to allow the cleaving to take place under stagnant conditions. The cleavage reaction can also take place in a flow mode, where the pH 6.0 buffer front is slowly applied to the column thereby allowing the cleaved desired product to accumulate at the buffer front as it passes through the column. The column can then be regenerated through one or more conventional means if desired. In principle, any physical configuration of affinity tag and immobilized ligand that allows the recovery of a purified protein from an affinity tag can be employed for the recovery of the product protein. This can include, but is not limited to, centrifugation to recover the affinity resin, the use of magnetic ligand-functionalized resins, membrane filtration to recover and wash the ligand-functionalized resin, and ligand-functionalized membranes that allow direct binding and washing of the tagged protein.

Variation in Ionic Strength for Intein-Mediated Cleavage

In yet another aspect of the present invention, in addition to the dependence of the cleavage reaction of the modified inteins or functional derivatives or homologs thereof comprising the TOPO® recognition sequence and/or a GATEWAY® recombination sites on temperature and/or pH, the modified inteins of the present invention are also capable of functioning in various buffers with different ionic strengths to achieve an efficient and simple purification process. Indeed, many buffers with varying ionic strengths, oxidative potential, and/or pH have been utilized. With respect to the parameter of ionic strength, various combinations of the following ranges of molar concentrations have been used in the column buffer with no adverse effect on the cleavage activity of the modified inteins or functional derivatives or homologs thereof: PIPES (0 to about 200 mM), Bis-Tris (0 to about 200 mM), Tris (0 to about 200 mM), EDTA (0 to about 10 mM), DTT (0 to about 10 mM), NaCl (0 to about 500 mM), and/or AMPD (0 to about 20 mM).

Variation in Half-Life for Intein-Mediated Cleavage

In yet another aspect of the present invention, the cleavage reaction of the modified inteins or functional derivatives or homologs thereof comprising the TOPO® recognition sequences and/or a GATEWAY® recombination sites of the present invention is characterized as an irreversible first-order reaction and is modeled as an exponential decay of precursor concentration. Consequently, one meaningful parameter is the “half-life” of the precursor, defined as the amount of time it takes for half of the precursor in a given sample to undergo cleavage. Slower cleaving is desired during protein expression and purification (long half-life), and fast cleaving is desired during the product protein cleaving and recovery step (short half-life). For practical applications involving the modified inteins or functional derivatives or homologs thereof comprising the TOPO® recognition sequence and/or a GATEWAY® recombination sites of the present invention, in one embodiment, the cleaving rate should preferably be slow (e.g., the half-life should be longer than about 70 hours during precursor expression and purification (thereby allowing less than about 10% yield loss during a typical 10 hour recovery and purification), but shorter than about 5 hours during cleaving and recovery of the product protein (allows 90% cleaving to take place overnight)). In another embodiment the cleaving half-life should preferably be slower (e.g., be longer than about 100 hours during precursor expression and purification (thereby allowing less than about 10% yield loss during a typical 10 hour recovery and purification), but shorter than about 2 hours during cleaving and recovery of the product protein (allows about 95% cleaving to take place overnight)). In any event, each purification method will depend on the requirements of the target protein being purified. The modified inteins of the present invention can more than adequately satisfy a range of cleaving rates at a range of temperatures, and are therefore widely applicable to a range of product proteins under a range of conditions. Significantly, the half-life of the modified inteins or functional derivatives or homologs thereof comprising the TOPO® recognition sequence and/or a GATEWAY® recombination sites is controlled by a combination of pH and temperature, and its activity can be varied by a factor of over about 10,000 using these parameter controls simultaneously from pH 8.5 and 4° C. (slowest possible cleaving) to pH 6.5 and 37° C. (approximately 10,000 times faster with typical half lives of less than 2 hours). Notwithstanding the representative examples of cleaving rates provide above for the modified inteins or functional derivatives or homologs thereof comprising the TOPO® recognition sequence and/or a GATEWAY® recombination sites of the present invention, alternate cleavage times (e.g., either substantially shorter and/or longer than those provided herein) are also possible depending upon the particular protein being purified.

Table 7 infra, provides some representative examples of data on cleavage kinetics and half-lives for a number of the modified inteins under a number of conditions with the aFGF test protein. One of skill in the art will appreciate that there is indeed a range of slow cleaving (typical half lives of hundreds of hours at pH 8.5 and 4° C.) to fast cleaving (typical half lives less than 2 to 8 hours at pH6.5 and 37° C.), and that specific cleaving rates between these two extremes can be acquired through combinations of pH and temperature between these two limits.

Recombination Sites and Recombinases for Use in the Invention

In yet another aspect of the present invention, the starting or product donor or expression vector molecules comprising nucleotide sequences encoding at least one modified intein or a functional derivative or homolog thereof may further comprise recombination sites and the corresponding recombinant proteins for these systems may also be used in accordance with the compositions and methods of the present invention. Representative non-limiting examples of recombination sites and recombination proteins for use in the invention include, inter alia, the FLP/FRT system from Saccharomyces cerevisiae, the resolvase family (e.g., (Tn3 resolvase, Hin, Gin and Cin), and IS231 and other Bacillus thuringiensis transposable elements. Other suitable recombination systems for use in the present invention include the XerC and XerD recombinases and the psi, dif and cer recombination sites in E. coli. Other suitable recombination sites may be found in U.S. Pat. No. 5,851,808 issued to Elledge and Liu which is specifically incorporated herein by reference. Preferred recombination proteins and mutant or modified recombination sites for use in the invention include those previously described in U.S. Pat. Nos. 5,888,732, 6,171,861, 6,143,557, 6,270,969 and 6,277,608, and co-pending U.S. application Ser. Nos. 09/438,358 (filed Nov. 12, 1999), 09/517,466 (filed Mar. 2, 2000), 09/695,065 (filed Oct. 25, 2000) and 09/732,914 (filed Dec. 11, 2000), the disclosures of all of which are specifically incorporated herein by reference in their entireties, as well as those associated with the Gateway® Cloning Technology available from Invitrogen Corporation (Carlsbad, Calif.).

Additional examples of preferred recombination proteins and mutant or modified recombination sites for use in the invention include MultiSite Gateway® system previously described in co-pending U.S. application Ser. No. 10/640,422 (filed Aug. 14, 2003), the entire disclosure of which is specifically incorporated herein by reference. MutliSite Gateway® is an extension of the Gateway® site-specific recombinational cloning system. The introduction of att sites with more than two specificities (e.g., by the addition of two att3 and att4 (in addition to att1 and att2 of the Gateway system) allows the simultaneous cloning of multiple DNA fragments in a defined order and orientation. MultiSite Gateway® applications are extensive and varied including but not limited to; the expression of multiple gene products from a single vector, addition of promoter/tag elements to the ends of standard GATEWAY™ Entry Clones (attL1/L2), construction of gene-targeting vectors, engineering and shuffling of protein coding domains, construction of synthetic operons, biological and biochemical pathway engineering and genome engineering.

Representative examples of a number of att recombination sites suitable for use in the compositions and methods of the invention are depicted in Table 2.

TABLE 2
Representative att recombination sites

| | | |
|---|---|---|
| attL5 | CAACTTTATTATACAAAGTTGGCA | (SEQ ID NO:14) |
| | GTTGAAATAATATGTTTCAACCGT | |
| attR5 | GTTCAACTTTATTATACAAAGTTGT | (SEQ ID NO:15) |
| | CAAGTTGAAATAATATGTTTCAACA | |
| attB11 | CAACTTTTCTATACAAAGTTGT | (SEQ ID NO:16) |
| | GTTGAAAAGATATGTTTCAACA | |
| attP11 | GTTCAACTTTTCTATACAAAGTTGGCA | (SEQ ID NO:17) |
| | CAAGTTGAAAAGATATGTTTCAACCGT | |
| attL11 | CAACTTTTCTATACAAAGTTGGCA | (SEQ ID NO:18) |
| | GTTGAAAAGATATGTTTCAACCGT | |
| attR11 | GTTCAACTTTTCTATACAAAGTTGT | (SEQ ID NO:19) |
| | CAAGTTGAAAAGATATGTTTCAACA | |
| attB17 | CAACTTTTGTATACAAAGTTGT | (SEQ ID NO:20) |
| | GTTGAAAACATATGTTTCAACA | |
| attP17 | GTTCAACTTTTGTATACAAAGTTGGCA | (SEQ ID NO:21) |
| | CAAGTTGAAAACATATGTTTCAACCGT | |
| attL17 | CAACTTTTGTATACAAAGTTGGCA | (SEQ ID NO:22) |
| | GTTGAAAACATATGTTTCAACCGT | |
| attR17 | GTTCAACTTTTGTATACAAAGTTGT | (SEQ ID NO:23) |
| | CAAGTTGAAAACATATGTTTCAACA | |
| attB19 | CAACTTTTTCGTACAAAGTTGT | (SEQ ID NO:24) |
| | GTTGAAAAAGCATGTTTCAACA | |
| attP19 | GTTCAACTTTTTCGTACAAAGTTGGCA | (SEQ ID NO:25) |
| | CAAGTTGAAAAAGCATGTTTCAACCGT | |
| attL19 | CAACTTTTTCGTACAAAGTTGGCA | (SEQ ID NO:26) |
| | GTTGAAAAACATATGTTTCAACCGT | |
| attR19 | GTTCAACTTTTTCGTACAAAGTTGT | (SEQ ID NO:27) |
| | CAAGTTGAAAAAGCATGTTTCAACA | |
| attB0 | AGCCTGCTTTTTTATACTAACTTGAGC | (SEQ ID NO:28) |
| | TCGGACGAAAAAATATGATTGAACTCG | |
| attP0 | GTTCAGCTTTTTTATACTAAGTTGGCA | (SEQ ID NO:29) |
| | CAAGTCGAAAAAATATGATTCAACCGT | |
| attL0 | AGCCTGCTTTTTTATACTAAGTTGGCA | (SEQ ID NO:30) |
| | TCGGACGAAAAAATATGATTCAACCGT | |
| attR0 | GTTCAGCTTTTTTATACTAACTTGAGC | (SEQ ID NO:31) |
| | CAAGTCGAAAAAATATGATTGAACTCG | |
| attB1 | AGCCTGCTTTTTTGTACAAACTTGT | (SEQ ID NO:32) |
| | TCGGACGAAAAAATATGTTTGAACA | |
| attP1 | GTTCAGCTTTTTTGTACAAAGTTGGCA | (SEQ ID NO:33) |
| | CAAGTCGAAAAAACATGTTTCAACCGT | |
| attL1 | AGCCTGCTTTTTTGTACAAAGTTGGCA | (SEQ ID NO:34) |
| | TCGGACGAAAAAACATGTTTCAACCGT | |
| attR1 | GTTCAGCTTTTTTGTACAAACTTGT | (SEQ ID NO:35) |
| | CAAGTCGAAAAAACATGTTTGAACA | |
| attB2 | ACCCAGCTTTCTTGTACAAAGTGGT | (SEQ ID NO:36) |
| | TGGGTCGAAAGAATATGTTTCACCA | |
| attP2 | GTTCAGCTTTCTTGTACAAAGTTGGCA | (SEQ ID NO:37) |
| | CAAGTCGAAAGAACATGTTTCAACCGT | |
| attL2 | ACCCAGCTTTCTTGTACAAAGTTGGCA | (SEQ ID NO:38) |
| | TGGGTCGAAAGAACATGTTTCAACCGT | |
| attR2 | GTTCAGCTTTCTTGTACAAAGTGGT | (SEQ ID NO:39) |
| | CAAGTCGAAAGAACATGTTTGACCA | |
| attB5 | CAACTTTATTATACAAAGTTGT | (SEQ ID NO:40) |
| | GTTGAAATAATATGTTTCAACA | |
| attP5 | GTTCAACTTTATTATACAAAGTTGGCA | (SEQ ID NO:41) |
| | CAAGTTGAAATAATATGTTTCAACCGT | |
| attB20 | CAACTTTTTGGTACAAAGTTGT | (SEQ ID NO:42) |
| | GTTGAAAAACCATGTTTCAACA | |
| attP20 | GTTCAACTTTTTGGTACAAAGTTGGCA | (SEQ ID NO:43) |
| | CAAGTTGAAAAACCATGTTTCAACCGT | |
| attL20 | CAACTTTTTGGTACAAAGTTGGCA | (SEQ ID NO:44) |
| | GTTGAAAAACCATGTTTCAACCGT | |
| attR20 | GTTCAACTTTTTGGTACAAAGTTGT | (SEQ ID NO:45) |
| | CAAGTTGAAAAACCATGTTTCAACA | |
| attB21 | CAACTTTTTAATACAAAGTTGT | (SEQ ID NO:46) |
| | GTTGAAAAATTATGTTTCAACA | |
| attP21 | GTTCAACTTTTTAATACAAAGTTGGCA | (SEQ ID NO:47) |
| | CAAGTTGAAAAATTATGTTTCAACCGT | |
| attL21 | CAACTTTTTAATACAAAGTTGGCA | (SEQ ID NO:48) |
| | GTTGAAAAATTATGTTTCAACCGT | |
| attR21 | GTTCAACTTTTTAATACAAAGTTGT | (SEQ ID NO:49) |
| | CAAGTTGAAAAATTATGTTTCAACA | |

More particularly, the recombination sites for use in the nucleic acids of the invention may be any recognition sequence on a nucleic acid molecule which participates in a recombination reaction catalyzed or facilitated by recombination proteins as recited above in the afore-mentioned commonly owned issued patents and/or pending patent applications. In those embodiments of the present invention utilizing more than one recombination site, such recombination sites may be the same or different and may recombine with each other or may not recombine or not substantially recombine with each other. Recombination sites contemplated by the invention also include mutants, derivatives or variants of wild-type or naturally occurring recombination sites. Preferred recombination site modifications include those that enhance recombination, such enhancement selected from the group consisting of substantially (i) favoring integrative recombination; (ii) favoring excisive recombination; (iii) relieving the requirement for host factors; (iv) increasing the efficiency of co-integrate or product formation; and (v) increasing the specificity of co-integrate or product formation. Preferred modifications include those that enhance recombination specificity, remove one or more stop codons, and/or avoid hair-pin formation. Desired modifications can also be made to the recombination sites to include desired amino acid changes to the transcription or translation product (e.g., mRNA or protein) when translation or transcription occurs across the modified recombination site. Recombination sites that may be used in accordance with the invention include att sites, frt sites, dif sites, psi sites, cer sites, and lox sites or mutants, derivatives and variants thereof (or combinations thereof). Recombination sites contemplated by the invention also include portions of such recombination sites.

Topoisomerase Recognition Sites and Topoisomerases

In yet another aspect of the present invention, the starting or product donor or expression vector molecules comprising nucleotide sequences encoding at least one modified intein or a functional derivative or homolog thereof may further comprise topoisomerase recognition sequences and the corresponding topoisomerase proteins for these systems may also be used in accordance with the compositions and methods of the present invention. Representative non-limiting examples of topoisomerase recognition sequences and topoisomerase proteins for use in the invention include, inter alia, the e.g., a type IA, type IB, and/or type II topoisomerases (gyrases). Preferred topoisomerase recognition sequences and topoisomerase proteins for use in the invention include those previously described in co-pending U.S. application Ser. No. 10/005,876, filed Dec. 7, 2001, (U.S. Patent Publication 2003/0186233) and PCT/US01/45773, filed Dec. 7, 2001, the disclosures of all of which are specifically incorporated herein by reference in their entireties, as well as those associated with the Gateway® Cloning Technology available from Invitrogen Corporation (Carlsbad, Calif.).

In those embodiments in which the nucleotide sequences encoding modified inteins or functional derivatives or homologs thereof are adjacent to or flanking the one or more topoisomerase recognition sequences and the corresponding topoisomerase proteins and/or one or more recombination sites and the corresponding recombination proteins, the distance, in terms of the number of nucleotides, between recombination sites, topoisomerase recognition sites and nucleic acid sequences encoding modified inteins or functional derivatives or homologs thereof comprising one or more sequence tags (e.g., affinity tags) which reside in a nucleic acid molecule of the invention will vary with the particular application for which the nucleic acid molecule is to be used, but can, for example, be zero, one, two, three, four, five, six, seven, eight, nine, ten, eleven, twelve, thirteen, fourteen, fifteen, sixteen, twenty, twenty-five, thirty, forty, fifty, sixty, eighty, one hundred, one hundred fifty, two hundred, three hundred, five hundred, seven hundred, nine hundred, one thousand, etc., or more, nucleotides. Further, the distance, in terms of the number of nucleotides, between recombination sites and topoisomerase recognition sites which reside in a nucleic acid molecule encoding one or more modified inteins or functional derivatives or homologs thereof may fall within the following ranges: 0-10 nucleotides, 10-30 nucleotides, 20-50 nucleotides, 40-80 nucleotides, 70-100 nucleotides, 90-200 nucleotides, 120-400 nucleotides, 200-400 nucleotides, 200-1000 nucleotides, 200-2,000 nucleotides, etc.

Various embodiments of the topoisomerase-based cloning reactions may be used in the methods of the present invention. These topoisomerase-based cloning reactions may be referred to as either Directional TOPO Cloning, TOPO Tools, and TOPO Cloning depending upon the intended application. As described herein, the compositions, methods and kits of the invention may be prepared and carried out using a phage-lambda site-specific recombination system. Further, such compositions, methods and kits may be prepared and carried out using the GATEWAY™ Recombinational Cloning System and/or the TOPO® Cloning System and/or the pENTR Directional TOPO® Cloning System, all of which are available from Invitrogen Corporation (Carlsbad, Calif.). Invitrogen TOPO Cloning Protocol. Invitrogen Corporation Carlsbad, Calif. (For a more detailed description of the Directional TOPO Cloning, TOPO Tools, and TOPO Cloning technologies, see, U.S. Patent Publication 2003/0186233), the disclosure of which is specifically incorporated herein by reference in its entirety)

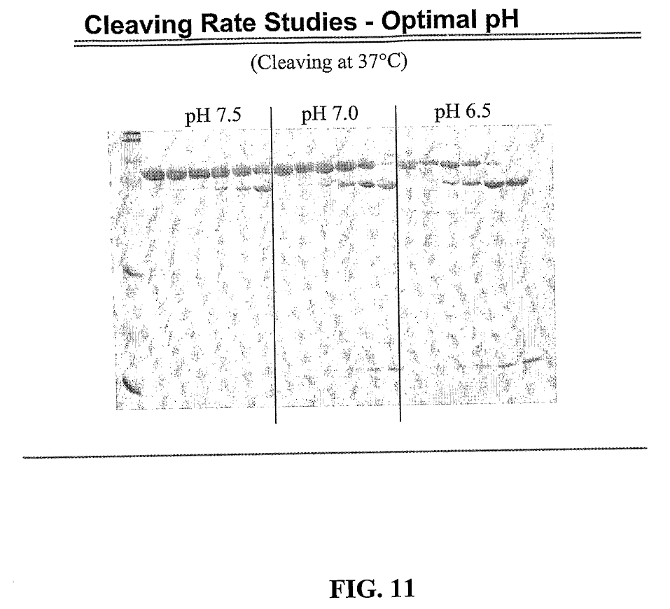

Each of these Topoisomerase based cloning reactions (e.g., Directional TOPO Cloning, TOPO Tools, and/or TOPO Cloning, see for example, page 14 of Appendix B, infra) may be used in the disclosed methods for generating a ds recombinant nucleic acid molecule covalently linked in one strand and, optionally, comprising one or more recombination sites. For example, one of the nucleic acid molecules of the present invention may have a topoisomerase attached to the 5′ terminus of one end such that, when this molecule, which has a 3′ overhang, is contacted with a second nucleic acid molecule having a substantially complementary 3′ overhang, under suitable conditions, the nucleotides comprising the 3′ overhangs can hybridize and the topoisomerases can catalyze ligation. For example, a first nucleic acid molecule having topoisomerase molecules linked to the 5′ terminus and 3′ terminus of two different ends of one nucleotide sequence, such that linkage of the first nucleic acid molecule to two other nucleotide sequences to generate a nucleic acid molecule which has one strand without any nicks and another strand with two nicks. In another example, a first nucleic acid molecule of the present invention having a topoisomerase molecule linked to the 5′ terminus of one end and a second nucleic acid molecule having a topoisomerase molecule linked to the 5′ terminus of one end, such that linkage of the first and second nucleic acid molecule to one other nucleotide sequence to generate a nucleic acid molecule which has one strand without any nicks and another strand with two nicks. In yet another example, one of the nucleic acid molecules to be linked has site-specific type IA topoisomerases attached to the 5′ terminus of both ends such that, when the nucleotide sequences are contacted the complementary 3′ overhangs can hybridize and the topoisomerases catalyze ligation. In yet another example, the methods of the present invention may be used to link three nucleic acid molecules together, using one nucleic acid molecule that is topoisomerase-charged with a type IA topoisomerase at a 5′ terminus and another nucleic acid molecule that is topoisomerase-charged with a type IB topoisomerase at a 3′ terminus of the opposite strand to be linked, such that when the nucleotide sequences are contacted the complementary 3′ overhangs can hybridize and the topoisomerases catalyze ligation. In yet another example, the methods of the present invention may be used to link three nucleic acid molecules together, in this case using one nucleic acid molecule that is topoisomerase-charged with a topoisomerase (e.g., a type IA or a type II topoisomerase) at a 5′ terminus and with a type IB topoisomerase at a 3′ terminus of the opposite strand, such that when the nucleotide sequences are contacted under suitable conditions, the complementary 3′ overhangs can hybridize and the topoisomerases catalyze ligation. Once nucleic acid molecules are joined by the methods described above, the resulting molecules may then be used in recombination reactions, such as those described elsewhere herein (for a more detailed description of the use of topoisomerase recognition sequences, see FIG. 11 of U.S. Patent Publication 2003/0186233), the disclosure of which is specifically incorporated herein by reference in its entirety).

The ds recombinant nucleic acid molecule generated using the methods of this aspect of the invention include those in which one strand (not both strands) is covalently linked at the ends to be linked (i.e. ds recombinant nucleic acid molecules generated using these methods contain a nick at each position where two ends were joined). These embodiments are particularly advantageous in that a polymerase can be used to replicate the ds recombinant nucleic acid molecule by initially replicating the covalently linked strand. For example, a thermostable polymerase such as a polymerase useful for performing an amplification reaction such as PCR can be used to replicate the covalently strand, whereas the strand containing the nick does not provide a suitable template for replication.

The present invention also provides methods of covalently ligating the ends of two different nucleic acid molecules or two ends of the same nucleic acid molecule, such that the product generated is ligated in both strands and, therefore, does not contain a nick. For example, one of the nucleic acid molecules has topoisomerase molecules attached to the 3′ terminus and the 5′ terminus of one end such that, when this molecule, which has a 5′ overhang, is contacted with a second nucleic acid molecule having a substantially complementary 5′ overhang, under suitable conditions, the nucleotides comprising the 5′ overhangs can hybridize and the topoisomerases can catalyze ligation of both strands of the nucleic acid molecules. In another example, each end of the nucleic acid molecules to be linked has a topoisomerase molecule attached to the 3′ terminus such that, when the nucleotide sequences are contacted under suitable conditions, nucleotides comprising the 5′ overhangs can hybridize and the topoisomerases catalyze ligation. In another example, the methods of the present invention may be used to link three nucleic acid molecules together via a nucleic acid molecule that is topoisomerase-charged at both termini of both ends. In these examples of topoisomerase-based cloning reactions, the ends of the nucleic acid molecules that are not being linked as having blunt ends. However, the substrate nucleic acid molecules utilized in these methods can have any ends as desired, including topoisomerase-charged ends, such that the ends can be ligated to each other, for example, to form circular molecules or to other nucleic acid molecules having an appropriate end, blunt ends, 5′ overhangs, 3′ overhangs, and the like, as desired. Once nucleic acid molecules are joined by the methods described above, the resulting molecules may then be used in recombination reactions, such as those described elsewhere herein (for a more detailed description of the use of topoisomerase recognition sequences, see FIG. 12 of U.S. Patent Publication 2003/0186233), the disclosure of which is specifically incorporated herein by reference in its entirety).

Representative examples of type IA topoisomerases include, inter alia, E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases (see Berger, Biochim. Biophys. Acta 1400:3-18, 1998; DiGate and Marians, J. Biol. Chem. 264:17924-17930, 1989; Kim and Wang, J. Biol. Chem. 267:17178-17185, 1992; Wilson et al., J. Biol. Chem. 275:1533-1540, 2000; Hanai et al., Proc. Natl. Acad. Sci., USA 93:3653-3657, 1996, U.S. Pat. No. 6,277,620, each of which is incorporated herein by reference). E. coli topoisomerase III, which is a type IA topoisomerase that recognizes, binds to and cleaves the sequence 5′-GCAACTT-3′, can be particularly useful in a method of the invention (Zhang et al., J. Biol. Chem. 270:23700-23705, 1995, which is incorporated herein by reference). A homolog, the traE protein of plasmid RP4, has been described by Li et al., J. Biol. Chem. 272:19582-19587 (1997) and can also be used in the practice of the invention. A DNA-protein adduct is formed with the enzyme covalently binding to the 5′-thymidine residue, with cleavage occurring between the two thymidine residues.

Representative examples of type IB topoisomerases include, inter alia, the nuclear type I topoisomerases present in all eukaryotic cells and those encoded by vaccinia and other cellular poxviruses (see Cheng et al., Cell 92:841-850, 1998, which is incorporated herein by reference). The eukaryotic type IB topoisomerases are exemplified by those expressed in yeast, Drosophila and mammalian cells, including human cells (see Caron and Wang, Adv. Pharmacol. 29B:271-297, 1994; Gupta et al., Biochim. Biophys. Acta 1262:1-14, 1995, each of which is incorporated herein by reference; see, also, Berger, supra, 1998). Viral type IB topoisomerases are exemplified by those produced by the vertebrate poxviruses (vaccinia, Shope fibroma virus, ORF virus, fowlpox virus, and molluscum contagiosum virus), and the insect poxvirus (Amsacta moorei entomopoxvirus) (see Shuman, Biochim. Biophys. Acta 1400:321-337, 1998; Petersen et al., Virology 230:197-206, 1997; Shuman and Prescott, Proc. Natl. Acad. Sci., USA 84:7478-7482, 1987; Shuman, J. Biol. Chem. 269:32678-32684, 1994; U.S. Pat. No. 5,766,891; PCT/US95/16099; PCT/US98/12372, each of which is incorporated herein by reference; see, also, Cheng et al., supra, 1998).

Representative examples of type II topoisomerases include, inter alia, bacterial gyrase, bacterial DNA topoisomerase IV, eukaryotic DNA topoisomerase II, and T-even phage encoded DNA topoisomerases (Roca and Wang, Cell 71:833-840, 1992; Wang, J. Biol. Chem. 266:6659-6662, 1991, each of which is incorporated herein by reference; Berger, supra, 1998).

Vectors for Use in the Compositions and Methods of the Invention

In accordance with the invention, any vector may be used to construct the vectors employing the modified intein nucleotide sequence based cloning and expression system of the present invention. In particular, vectors known in the art and those commercially available (and variants or derivatives thereof) may in accordance with the invention be engineered to include one or more recombination sites and/or topoisomerase sites flanking the, adjacent to, and/or embedded within the at least one modified intein nucleotide sequence or a derivative or homolog thereof for use in the methods of the invention. Such vectors may be obtained from, for example, Vector Laboratories Inc., Invitrogen Corp., Promega, Novagen, NEB, Clontech, Boehringer Mannheim, Pharmacia, EpiCenter, OriGenes Technologies Inc., Stratagene, Perkin Elmer, Pharmingen. Such vectors may then for example be used for cloning or subcloning nucleic acid molecules of interest. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts and the like.

Other vectors of interest include viral origin vectors (M13 vectors, bacterial phage .lambda. vectors, adenovirus vectors, and retrovirus vectors), high, low and adjustable copy number vectors, vectors which have compatible replicons for use in combination in a single host (pACYC184 and pBR322) and eukaryotic episomal replication vectors (pCDM8).

In accordance with the present invention, vectors are modified to further comprise one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags). Particular vectors which may be modified by the addition of the at least one modified intein nucleotide sequence or a derivative or homolog thereof described herein include prokaryotic expression vectors such as pcDNA II, pSL301, pSE280, pSE380, pSE420, pTrcHisA, B, and C, pRSET A, B, and C (Invitrogen Corp.), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and pProEx-HT (Invitrogen Corp.) and variants and derivatives thereof. Vector donors can also be made from eukaryotic expression vectors such as pFastBac, pFastBac HT, pFastBac DUAL, pSFV, and pTet-Splice (Invitrogen Corp.), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and pYES2, pAC360, pBlueBacHis A, B, and C, pVL1392, pBsueBacIII, pCDM8, pcDNA1, pZeoSV, pcDNA3 pREP4, pCEP4, and pEBVHis (Invitrogen Corp.).

Other vectors of particular interest that may be modified to further comprise one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags), include, for example, pUC18, pUC19, pBlueScript, pSPORT, cosmids, phagemids, YAC's (yeast artificial chromosomes), BAC's (bacterial artificial chromosomes), P1 (E. coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen Corp.), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen Corp.) and variants or derivatives thereof.

Additional vectors of interest that may be modified to further comprise comprise one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags), include, for example, pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZ.alpha., pGAPZ, pGAPZ.alpha., pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe,SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen Corp.; pCMV.beta., pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, .lambda.gt10, .lambda.gt11, pWE15, and .lambda.TriplEx from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS+/−, pBC KS+/−, pBC SK+/−, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMC1neo, pMC1neo Poly A, pOG44, pOG45, pFRT.beta.GAL, pNEO.beta.GAL, pRS403, pRS404, pRS405, pRS406, pRS413, pRS414, pRS415, and pRS416 from Stratagene.

The invention further includes nucleic acid molecules (e.g., vectors) modified to further comprise one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags), which nucleic acid molecules (e.g., vectors) are suitable for propagation in more than one (e.g., two, three, four, etc.) type of host cell, as well as methods for making such nucleic acid molecules. For example, these nucleic acid molecules may be capable of propagating in bacterial host cells (e.g., Escherichia coli) and mammalian host cells (e.g., human cells such as 293 cells). Such vectors are often referred to as “shuttle vectors”. Thus, nucleic acid molecules used in the present invention may comprise one or more origins of replication (ORIs), and/or one or more positive or negative selectable markers. In some embodiments, the nucleic acid molecules may comprise two or more ORIs which are capable of functioning in different organisms (e.g., one which functions in prokaryotes and one which functions in eukaryotes). For example, a nucleic acid may have an ORI that functions in one or more prokaryotes (e.g., E. coli, Bacillus, etc.) and another that functions in one or more eukaryotes (e.g., yeast, insect, mammalian cells, etc.). Selectable markers may likewise be included in nucleic acid molecules of the invention to allow for selection of desired molecules in different organisms. For example, a nucleic acid molecule may comprise multiple selectable markers, one or more of which functions in prokaryotes and one or more of which functions in eukaryotes.

As noted above nucleic acid molecules of the invention may contain one or more positive or negative selectable markers. When the nucleic acid molecules which are suitable for propagation in more than one type of host cell, these molecules will often contain two or more positive or negative selectable markers. Of course, this may not be the case when the positive or negative selectable markers is capable of functioning in more than one cell type. One example of such a selectable marker is the blastocidin S resistance marker, which allows for the positive or negative selection of both prokaryotic and eukaryotic cells which express the marker. Examples of positive or negative selectable markers which can be used in prokaryotic cells include those which confer resistance to ampicillin, kanamycin, spectinomycin, chloramphenicol, and tetracycline. Examples of positive or negative selectable markers which can be used in eukaryotic cells include those which confer resistance to hygromycin B, ZEOCIN™ (Invitrogen Corporation, Carlsbad, Calif.), and GENTICIN® (Invitrogen Corporation, Carlsbad, Calif.). Nucleic acid molecules and methods of the invention may contain and/or employ one or more of the above positive or negative selectable markers, as well as additional selectable markers.

According to the invention, vectors comprising comprise one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags), may be produced by one of ordinary skill in the art without resorting to undue experimentation using standard molecular biology methods. For example, vectors of the invention, as well as vectors suitable for use in methods of the invention, may be engineered by introducing one or more of the nucleic acid molecules encoding one or more recombination sites (or mutants, fragments, variants or derivatives thereof) and/or topoisomerase sites (or mutants, fragments, variants or derivatives thereof) flanking the. adjacent to, and/or embedded within the at least one modified intein nucleotide sequence or a derivative or homolog thereof into one or more of the vectors described herein, according to the methods described, for example, in Maniatis et al., Molecular Cloning: A Laboratory Manual, Cold Spring Harbor Laboratory, Cold Spring Harbor, N.Y. (1982).

The invention thus also includes methods of modifying existing vectors (e.g. selection, donor, expression vectors, etc.) to provide for insertion of the one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags) at least one modified intein nucleotide sequence or a derivative or homolog thereof so as to generate modified vectors containing the at least one modified intein nucleotide sequence or a derivative or homolog thereof, as well as methods of using such vectors after having been modified.

Thus, in a related aspect of the invention, vectors may be engineered to contain, in addition to one or more nucleic acid molecules encoding one or more recombination sites (or mutants, fragments, variants or derivatives thereof) and/or topoisomerase sites (or mutants, fragments, variants or derivatives thereof) flanking the at least one modified intein nucleotide sequence or a derivative or homolog thereof, one or more additional physical or functional nucleotide sequences, such as those encoding one or more multiple cloning sites, one or more transcription termination sites, one or more transcriptional regulatory sequences (e.g., one or more promoters, enhancers, or repressors), one or more selection markers or modules, one or more genes or portions of genes encoding a protein or polypeptide of interest, one or more translational signal sequences, one or more nucleotide sequences encoding a fusion partner protein or peptide (e.g., GST, His.sub.6 or thioredoxin), one or more origins of replication, and one or more 5′ or 3′ polynucleotide tails (particularly a poly-G tail). According to this aspect of the invention, the one or more recombination site nucleotide sequences (or portions thereof) and/or topoisomerase sites (or portions thereof) flanking the, adjacent to, and/or embedded within the at least one modified intein nucleotide sequence or a derivative or homolog thereof may optionally be operably linked to the one or more additional physical or functional nucleotide sequences described herein.

Vectors according to this aspect of the invention include, but are not limited to: pENTR1A, pENTR2B, pENTR3c, pENTR4, pENTR5, pENTR6, pENTR7, pENTR8, pENTR9, pENTR10, pENTR11, pDEST1, pDEST2, pDEST3, pDEST4, pDEST5, pDEST6, pDEST7, pDEST8, pDEST9, pDEST10, pDEST11, pDEST12.2 (also known as pDEST12), pDEST13, pDEST14, pDEST15, pDEST16, pDEST17, pDEST18, pDEST19, pDEST20, pDEST21, pDEST22, pDEST23, pDEST24, pDEST25, pDEST26, pDEST27, pEXP501 (also known as pCMVSPORT6.0, pDONR201 (FIGS. 26A-26C), pDONR202, pDONR203, pDONR204, pDONR205, pDONR206, pDONR212, pDONR212(F), pDONR212(R), pMAB58, pMAB62, pDEST28, pDEST29, pDEST30, pDEST31, pDEST32, pDEST33, pDEST34, pDONR207, pMAB85, pMAB86, a number of which are described in PCT Publication WO 00/52027 (the entire disclosure of which is incorporated herein by reference), and fragments, mutants, variants, and derivatives of each of these vectors. However, it will be understood by one of ordinary skill that the present invention also encompasses other vectors not specifically designated herein, which comprise one or more of the isolated nucleic acid molecules used in the invention encoding one or more one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags), and which may further comprise one or more additional physical or functional nucleotide sequences described herein which may optionally be operably linked to the one or more nucleic acid molecules encoding one or more recombination sites and the corresponding recombination proteins and/or topoisomerase recognition sequences and the corresponding topoisomerase proteins flanking the, adjacent to, and/or embedded within the at least one modified intein nucleotide sequence or a derivative or homolog thereof, and wherein the nucleotide sequence further encodes a protein of interest that has been modified to contain one or more self-cleaving sequence tags (e.g., affinity tags). Such additional vectors may be produced by one of ordinary skill according to the guidance provided in the present specification.

As one skilled in the art would recognize, in many instances when nucleic acid molecules of the invention are introduced into organisms, it will be desirable to operably link sequences which encode expression products to tissue-specific transcriptional regulatory sequences (e.g., tissue-specific promoters) where production of the expression product is desired. Such promoters can be used to facilitate production of these expression products in desired tissues. A considerable number of tissue-specific promoters are known in the art.

Host Cells and Cell Lines for Propagating Modified Intein-Containing Vectors

The invention also relates to host cells comprising one or more of the nucleic acid molecules or vectors used in, selected and/or isolated by the invention, particularly those nucleic acid molecules and vectors described in detail herein. Representative host cells that may be used according to this aspect of the invention include, but are not limited to, bacterial cells, yeast cells, plant cells and animal cells. Bacterial host cells suitable for use with the invention include Escherichia spp. cells (particularly E. coli cells and most particularly E. coli strains DH10B, Stb12, DH5.alpha., DB3, DB3.1 (e.g., E. coli LIBRARY EFFICIENCY® DB3.1® Competent Cells; Invitrogen Corp., Carlsbad, Calif.), DB4 and DB5; see U.S. application Ser. No. 09/518,188, filed on Mar. 2, 2000, the disclosure of which is incorporated by reference herein in its entirety), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salinonella spp. cells (particularly S. typhimurium and S. typhi cells). Animal host cells suitable for use with the invention include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly CHO, COS, VERO, BHK and human cells). Yeast host cells suitable for use with the invention include Saccharomyces cerevisiae cells and Pichia pastoris cells. These and other suitable host cells are available commercially, for example from Invitrogen Corp., Carlsbad, Calif., American Type Culture Collection (Manassas, Va.), and Agricultural Research Culture Collection (NRRL; Peoria, Ill.).

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, electroporation, transfection, and transformation. The nucleic acid molecules and/or vectors of the invention may be introduced alone or in conjunction with other the nucleic acid molecules and/or vectors and/or proteins, peptides or RNAs. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Thus nucleic acid molecules of the invention may contain and/or encode one or more packaging signals (e.g., viral packaging signals which direct the packaging of viral nucleic acid molecules). Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

### Methods of Using the Compositions of the Invention

Overview of Cloning Strategies or Approaches

Thus, in one aspect of the invention, there are at least two approaches for cloning and/or expressing nucleotides sequences of interest using the modified intein compositions and methods of the present invention. These approaches can be summarized as follows: (1) Conventional-based nucleic acid cloning methods; and (2) recombination-based cloning methods. In each instance, the at least one modified intein nucleotide sequence or a functional derivative or homolog thereof may be used in conjunction with a negative selection marker (e.g., ccdB) in the conventional and recombination-based cloning and expression systems. The presence of the ccdB gene allows negative selection of the vector (e.g., donor, entry, destination or expression vectors) molecules in E. coli following ligation/recombination and transformation.

Thus, in one aspect of the invention, the newly created population of molecules created by the standard recombinant cloning methods may be preferentially selected and thus separated or isolated from the original molecules (e.g., target molecules, and first and second population molecules) and from undesired product molecules (e.g., parental vector molecules). Such selective systems may be accomplished by positive and/or negative selection. One or more toxic genes (e.g., two, three, four, five seven, ten, etc.) are used according to the invention in such a negative selection scheme. Such selection may also be accomplished by assaying or selecting for the presence of a desired nucleic acid fusion (PCR with diagnostic primers) and/or the presence of a desired activity of a protein encoded by the desired nucleic acid fusion construct.

In an additional aspect, the newly created population of molecules (e.g., the third population) created by the combinatorial methods may be preferentially selected and thus separated or isolated from the original molecules (e.g., target molecules, and first and second population molecules) and from undesired product molecules (e.g., cointegrates and/or byproduct molecules). Such selective may be accomplished by positive and/or negative selection. One or more toxic genes (e.g., two, three, four, five seven, ten, etc.) are used according to the invention in such negative selection schemes. Such selection may also be accomplished by assaying or selecting for the presence of a desired nucleic acid fusion (PCR with diagnostic primers) and/or the presence of a desired activity of a protein encoded by the desired nucleic acid fusion.

Additionally, combinations of selection of positive and/or negative selection and the desired fusion product (nucleic acid and/or protein) may also be used in the invention. Thus, the invention provides a means for selecting a population of nucleic acid product molecules (or even a specific class of product molecules or specific product molecule) created by the standard recombinant cloning methods or recombinational cloning methods described herein and selecting against a population of nucleic acid product molecules (e.g., Insert Donors, Vector Donors and Cointegrates) or, in similar fashion, selecting for a population of nucleic acid product molecules (e.g., Insert Donors, Vector Donors, Byproducts and/or Cointegrates) and selecting against a population of nucleic acid product molecules (See FIG. 1).

Those skilled in the art will appreciated that in this situation, cointegrate molecules, other than the one shown in FIG. 1, may be produced. For example, cointegrates comprising a segment A and a segment B Insert Donor molecule may be formed. In addition, cointegrates comprising segment A and/or segment B Insert Donor molecules and a Vector Donor molecule may be formed. The selection methods of the present invention permit selection against the Insert Donor molecules and against the various cointegrate molecules and for the newly created population of hybrid molecules which may be referred to as a population of Product molecules. Conversely, the selection methods may be designed to permit selection against Products and for Insert/Vector Donors, Byproducts, and/or Cointegrates.

Types of Selection Vectors for Use with the Compositions of the Invention

Negative Selection Vectors for Use with the Compositions of the Invention

In certain embodiments of the present invention, the at least one intein nucleotide sequence or a functional derivative or homolog thereof may be used in conjunction with a negative selection marker (e.g., ccdB) in both conventional and recombination-based cloning and expression systems.

In one particular embodiment of a modified intein nucleotide sequence or a functional derivative or homolog thereof-containing vector, the vector nucleic acid molecules of the invention may further comprise a negative selection marker (e.g., ccdB) operatively linked to an appropriate promotor/operator region, together with one or more origins of replication, and one or more selectable markers, wherein the negative selection marker further contains one or more cloning sites with one or more unique restriction enzyme recognition sites such that a nucleic acid molecule of interest further modified to contain at least one modified intein nucleotide sequence with one or more sequence tags may be cloned into one or more cloning sites of the negative selection marker nucleotide sequence resulting in disruption of the open reading frame of the negative selection marker and/or disruption of the open reading frame of the negative selection marker nucleotide sequence from the negative selection marker promoter/operator region. Subsequent expression of the protein of interest, followed by the self-splicing reaction of the modified intein under suitable conditions of pH and temperature serves to excise the modified intein with the one or more sequence tags thereby facilitating recovery of the protein of interest.

In one particular embodiment of a modified intein nucleotide sequence or a functional derivative or homolog thereof-containing vector, the vector nucleic acid molecules of the invention may further comprise a negative selection marker nucleotide sequence fusion (e.g., C-terminal/N-terminal fusions) construct or a functional derivative or homolog thereof operatively linked to the negative selection marker promotor/operator region, together with one or more origins of replication, and one or more selectable markers, wherein the negative selection marker nucleotide sequence fusion construct or a functional derivative or homolog thereof contains one or more cloning sites with one or more unique restriction enzyme recognition sites such that a nucleic acid molecule of interest modified to contain at least one modified intein nucleotide sequence with one or more sequence tags may be cloned into one or more of the cloning sites of the negative selection marker nucleotide sequence fusion construct thereby resulting in disruption of the open reading frame of the negative selection marker nucleotide sequence fusion construct and/or disruption of the open reading frame of the negative selection marker nucleotide sequence fusion construct from the negative selection marker promoter/operator region. Subsequent expression of the protein of interest, followed by the self-splicing reaction of the modified intein under suitable conditions of pH and temperature serves to excise the modified intein with the one or more sequence tags thereby facilitating recovery of the protein of interest.

In another embodiment of a negative selection-based donor vector of the present invention, a starting donor or expression vector product nucleic acid molecule is provided further comprising at least one modified intein nucleic acid sequence with the one or more sequence tags and further comprising one or more recombination sites, and a nucleic acid molecule encoding at least one negative selection marker (e.g., ccdB) located between two recombination sites and the at least one modified intein nucleotide sequence.

In this particular embodiment, by conducting a recombination reaction such that all or a portion of the nucleic acid molecules of interest with recombination sites in a first population is recombined with one or more molecules from the donor or expression vector product nucleic acid molecules, a third population of hybrid nucleic acid molecules is formed. Through resolution of the resultant cointegrates, the nucleic acid molecule of interest is cloned between the one or more recombination sites and the at least one modified intein nucleotide sequence. In this embodiment, the presence of the lethal ccdB gene or a functional derivative or homolog thereof is serving as a negative selection marker in that transformation of bacterial cells susceptible to the lethal effects of the ccdB toxin with non-recombinant parental donor or expression vector starting molecules, cointegrates, or donor or expression vector byproduct molecules results in cell death. Only true recombinant plasmid vector clones containing the nucleic acid of interest and further comprising recombination sites and at least one modified intein nucleotide sequence with the one or more sequence tags, without the ccdB nucleotide sequence, will be capable of growth as a result of the recombinase-mediated excision of the ccdB gene. Once again, due to the lethality of the ccdB gene, for this recombinational-based cloning system, the starting donor or expression vector nucleic acid molecule harboring the ccdB nucleotide sequence will often be propagated in cells resistant to the lethal effects of the ccdB toxin. Subsequent expression of the protein of interest, followed by the self-splicing reaction of the modified intein under suitable conditions of pH and temperature serves to excise the modified intein with the one or more sequence tags thereby facilitating recovery of the protein of interest.

Thus, by way of specific reference to FIG. 5, and for illustration purposes only, FIG. 5 depicts a prophetic pET-GWMIT vector nucleic acid molecule which comprises an att recombination site (for example, an attB1 site in this case) and one modified intein nucleotide sequence. The vector is approximately 6.2 Kbp in size and contains, inter alia, an Sp6 promoter/priming site, a T7 promoter/priming site, and a pBR322 origin as well as an ampicillin resistance gene (which may be substituted with other selectable antibiotic resistance genes such as, for example, kanamycin, spectinomycin). The nucleotide sequence of the pET-GWMIT vector depicted in FIG. 5 is shown in Table 3.

TABLE 3
Nucleotide Sequence of pETGWMIT (SEQ ID NO:9)

| | |
|---|---|
| 1 | caaggagatg gcgcccaaca gtcccccggc cacggggcct gccaccatac ccacgccgaa |
| 61 | acaagcgctc atgagcccga agtggcgagc ccgatcttcc ccatcggtga tgtcggcgat |
| 121 | ataggcgcca gcaaccgcac ctgtggcgcc ggtgatgccg gccacgatgc gtccggcgta |
| 181 | gaggatcgag atctcgatcc cgcgaaatta atacgactca ctatagggga attgtgagcg |
| 241 | gataacaatt cccctctaga aataattttg tttaacttta agaaggaatt cgccctcgca |
| 301 | gagggcactc ggatcttcga tccggtcacc ggtacaacgc atcgcatcga ggatgttgtc |
| 361 | ggtgggcgca agcctattca tgtcgtggct gctgccaagg acggaacgct gcatgcgcgg |
| 421 | cccgtggtgt cctggttcga ccagggaacg cgggatgtga tcgggttgcg gatcgccggt |
| 481 | ggcgccatcc tgtgggcgac acccgatcac aaggtgctga cagagtacgg ctggcgtgcc |
| 541 | gccggggaac tccgcaaggg agacagggtg gcgcaaccgc gacgcttcga tggattcggt |
| 601 | gacagtgcgc cgattccgac aagtttgtac aaaaaagcag gcagcgcgcg cgtgcaggcg |
| 661 | ctcgcggatg ccctggatga caaattcctg cacgacatgc tggcggaaga actccgctat |
| 721 | tccgtgatcc gagaagtgct gccaacgcgg cgggcacgaa cgttcggcct cgaggaactg |
| 781 | cacaccctcg tcgccgaagg ggttcttgta cacaacnnnn nnnnnnnnnn nnnnnnnnnn |
| 841 | ggtaagccta tccctaaccc tctcctcggt ctcgattcta cgcgtaccgg tcatcatcac |
| 901 | catcaccatt gagtttgatc cggctgctaa caaagcccga aaggaagctg agttggctgc |
| 961 | tgccaccgct gagcaataac tagcataacc ccttggggcc tctaaacggg tcttgagggg |
| 1021 | ttttttgctg aaaggaggaa ctatatccgg atatcccgca agaggcccgg cagtaccggc |
| 1081 | ataaccaagc ctatgcctac agcatccagg gtgacggtgc cgaggatgac gatgagcgca |
| 1141 | ttgttagatt tcatacacgg tgcctgactg cgttagcaat ttaactgtga taaactaccg |
| 1201 | cattaaagct tatcgatgat aagctgtcaa acatgagaat taattcttga agacgaaagg |
| 1261 | gcctcgtgat acgcctattt ttataggtta atgtcatgat aataatggtt tcttagacgt |
| 1321 | caggtggcac ttttcgggga aatgtgcgcg gaacccctat ttgtttattt ttctaaatac |
| 1381 | attcaaatat gtatccgctc atgagacaat aaccctgata aatgcttcaa taatattgaa |
| 1441 | aaaggaagag tatgagtatt caacatttcc gtgtcgccct tattcccttt tttgcggcat |
| 1501 | tttgccttcc tgtttttgct cacccagaaa cgctggtgaa agtaaaagat gctgaagatc |
| 1561 | agttgggtgc acgagtgggt tacatcgaac tggatctcaa cagcggtaag atccttgaga |
| 1621 | gttttcgccc cgaagaacgt tttccaatga tgagcacttt taaagttctg ctatgtggcg |
| 1681 | cggtattatc ccgtgttgac gccgggcaag agcaactcgg tcgccgcata cactattctc |
| 1741 | agaatgactt ggttgagtac tcaccagtca cagaaaagca tcttacggat ggcatgacag |
| 1801 | taagagaatt atgcagtgct gccataacca tgagtgataa cactgcggcc aacttacttc |
| 1861 | tgacaacgat cggaggaccg aaggagctaa ccgctttttt gcacaacatg ggggatcatg |
| 1921 | taactcgcct tgatcgttgg gaaccggagc tgaatgaagc cataccaaac gacgagcgtg |
| 1981 | acaccacgat gcctgcagca atggcaacaa cgttgcgcaa actattaact ggcgaactac |
| 2041 | ttactctagc ttcccggcaa caattaatag actggatgga ggcggataaa gttgcaggac |
| 2101 | cacttctgcg ctcggccctt ccggctggct ggtttattgc tgataaatct ggagccggtg |
| 2161 | agcgtgggtc tcgcggtatc attgcagcac tggggccaga tggtaagccc tcccgtatcg |
| 2221 | tagttatcta cacgacgggg agtcaggcaa ctatggatga acgaaataga cagatcgctg |
| 2281 | agataggtgc ctcactgatt aagcattggt aactgtcaga ccaagtttac teatatatac |
| 2341 | tttagattga tttaaaactt catttttaat ttaaaaggat ctaggtgaag atcctttttg |
| 2401 | ataatctcat gaccaaaatc ccttaacgtg agttttcgtt ccactgagcg tcagaccccg |
| 2461 | tagaaaagat caaaggatct tcttgagatc ctttttttct gcgcgtaatc tgctgcttgc |
| 2521 | aaacaaaaaa accaccgcta ccagcggtgg tttgtttgcc ggatcaagag ctaccaactc |
| 2581 | tttttccgaa ggtaactggc ttcagcagag cgcagatacc aaatactgtc cttctagtgt |
| 2641 | agccgtagtt aggccaccac ttcaagaact ctgtagcacc gcctacatac ctcgctctgc |
| 2701 | taatcctgtt accagtggct gctgccagtg gcgataagtc gtgtcttacc gggttggact |
| 2761 | caagacgata gttaccggat aaggcgcagc ggtcgggctg aacggggggt tcgtgcacac |
| 2821 | agcccagctt ggagcgaacg acctacaccg aactgagata cctacagcgt gagctatgag |
| 2881 | aaagcgccac gcttcccgaa gggagaaagg cggacaggta tccggtaagc ggcagggtcg |
| 2941 | gaacaggaga gcgcacgagg gagcttccag ggggaaacgc ctggtatctt tatagtcctg |
| 3001 | tcgggtttcg ccacctctga cttgagcgtc gatttttgtg atgctcgtca ggggggcgga |
| 3061 | gcctatggaa aaacgccagc aacgcggcct ttttacggtt cctggccttt tgctggcctt |
| 3121 | ttgctcacat gttctttcct gcgttatccc ctgattctgt ggataaccgt attaccgcct |
| 3181 | ttgagtgagc tgataccgct cgccgcagcc gaacgaccga gcgcagcgag tcagtgagcg |
| 3241 | aggaagcgga agagcgcctg atgcggtatt ttctccttac gcatctgtgc ggtatttcac |
| 3301 | accgcaatgg tgcactctca gtacaatctg ctctgatgcc gcatagttaa gccagtatac |
| 3361 | actccgctat cgctacgtga ctgggtcatg gctgcgcccc gacacccgcc aacacccgct |
| 3421 | gacgcgccct gacgggcttg tctgctcccg gcatccgctt acagacaagc tgtgaccgtc |
| 3481 | tccgggagct gcatgtgtca gaggttttca ccgtcatcac cgaaacgcgc gaggcagctg |
| 3541 | cggtaaagct catcagcgtg gtcgtgaagc gattcacaga tgtctgcctg ttcatccgcg |
| 3601 | tccagctcgt tgagtttctc cagaagcgtt aatgtctggc ttctgataaa gcgggccatg |
| 3661 | ttaagggcgg ttttttcctg tttggtcact gatgcctccg tgtaaggggg atttctgttc |
| 3721 | atgggggtaa tgataccgat gaaacgagag aggatgctca cgatacgggt tactgatgat |
| 3781 | gaacatgccc ggttactgga acgttgtgag ggtaaacaac tggcggtatg gatgcggcgg |
| 3841 | gaccagagaa aaatcactca gggtcaatgc cagcgcttcg ttaatacaga tgtaggtgtt |
| 3901 | ccacagggta gccagcagca tcctgcgatg cagatccgga acataatggt gcagggcgct |
| 3961 | gacttccgcg tttccagact ttacgaaaca cggaaaccga agaccattca tgttgttgct |
| 4021 | caggtcgcag acgttttgca gcagcagtcg cttcacgttc gctcgcgtat cggtgattca |
| 4081 | ttctgctaac cagtaaggca accccgccag cctagccggg tcctcaacga caggagcacg |
| 4141 | atcatgcgca cccgtggcca ggacccaacg ctgcccgaga tgcgccgcgt gcggctgctg |
| 4201 | gagatggcgg acgcgatgga tatgttctgc caagggttgg tttgcgcatt cacagttctc |
| 4261 | cgcaagaatt gattggctcc aattcttgga gtggtgaatc cgttagcgag gtgccgccgg |
| 4321 | cttccattca ggtcgaggtg gcccggctcc atgcaccgcg acgcaacgcg gggaggcaga |
| 4381 | caaggtatag ggcggcgcct acaatccatg ccaacccgtt ccatgtgctc gccgaggcgg |
| 4441 | cataaatcgc cgtgacgatc agcggtccaa tgatcgaagt taggctggta agagccgcga |
| 4501 | gcgatccttg aagctgtccc tgatggtcgt catctacctg cctggacagc atggcctgca |
| 4561 | acgcgggcat cccgatgccg ccggaagcga gaagaatcat aatggggaag gccatccagc |
| 4621 | ctcgcgtcgc gaacgccagc aagacgtagc ccagcgcgtc ggccgccatg ccggcgataa |
| 4681 | tggcctgctt ctcgccgaaa cgtttggtgg cgggaccagt gacgaaggct tgagcgaggg |
| 4741 | cgtgcaagat tccgaatacc gcaagcgaca ggccgatcat cgtcgcgctc cagcgaaagc |
| 4801 | ggtcctcgcc gaaaatgacc cagagcgctg ccggcacctg tcctacgagt tgcatgataa |
| 4861 | agaagacagt cataagtgcg gcgacgatag tcatgccccg cgcccaccgg aaggagctga |
| 4921 | ctgggttgaa ggctctcaag ggcatcggtc gagatcccgg tgcctaatga gtgagctaac |
| 4981 | ttacattaat tgcgttgcgc tcactgcccg ctttccagtc gggaaacctg tcgtgccagc |
| 5041 | tgcattaatg aatcggccaa cgcgcgggga gaggcggttt gcgtattggg cgccagggtg |
| 5101 | gtttttcttt tcaccagtga gacgggcaac agctgattgc ccttcaccgc ctggccctga |
| 5161 | gagagttgca gcaagcggtc cacgctggtt tgccccagca ggcgaaaatc ctgtttgatg |
| 5221 | gtggttaacg gcgggatata acatgagctg tcttcggtat cgtcgtatcc cactaccgag |
| 5281 | atatccgcac caacgcgcag cccggactcg gtaatggcgc gcattgcgcc cagcgccatc |
| 5341 | tgatcgttgg caaccagcat cgcagtggga acgatgccct cattcagcat ttgcatggtt |
| 5401 | tgttgaaaac cggacatggc actccagtcg ccttcccgtt ccgctatcgg ctgaatttga |
| 5461 | ttgcgagtga gatatttatg ccagccagcc agacgcagac gcgccgagac agaacttaat |
| 5521 | gggcccgcta acagcgcgat ttgctggtga cccaatgcga ccagatgctc cacgcccagt |
| 5581 | cgcgtaccgt cttcatggga gaaaataata ctgttgatgg gtgtctggtc agagacatca |
| 5641 | agaaataacg ccggaacatt agtgcaggca gcttccacag caatggcatc ctggtcatcc |
| 5701 | agcggatagt taatgatcag cccactgacg cgttgcgcga gaagattgtg caccgccgct |
| 5761 | ttacaggctt cgacgccgct tcgttctacc atcgacacca ccacgctggc acccagttga |
| 5821 | tcggcgcgag atttaatcgc cgcgacaatt tgcgacggcg cgtgcagggc cagactggag |
| 5881 | gtggcaacgc caatcagcaa cgactgtttg cccgccagtt gttgtgccac gcggttggga |
| 5941 | atgtaattca gctccgccat cgccgcttcc actttttccc gcgttttcgc agaaacgtgg |
| 6001 | ctggcctggt tcaccacgcg ggaaacggtc tgataagaga caccggcata ctctgcgaca |
| 6061 | tcgtataacg ttactggttt cacattcacc accctgaatt gactctcttc cgggcgctat |
| 6121 | catgccatac cgcgaaaggt tttgcgccat tcgatggtgt ccgggatctc gacgctctcc |
| 6181 | cttatgcgac tcctgcatta ggaagcagcc cagtagtagg ttgaggccgt tgagcaccgc |
| 6241 | cgccgcaagg aatggtgcat g |

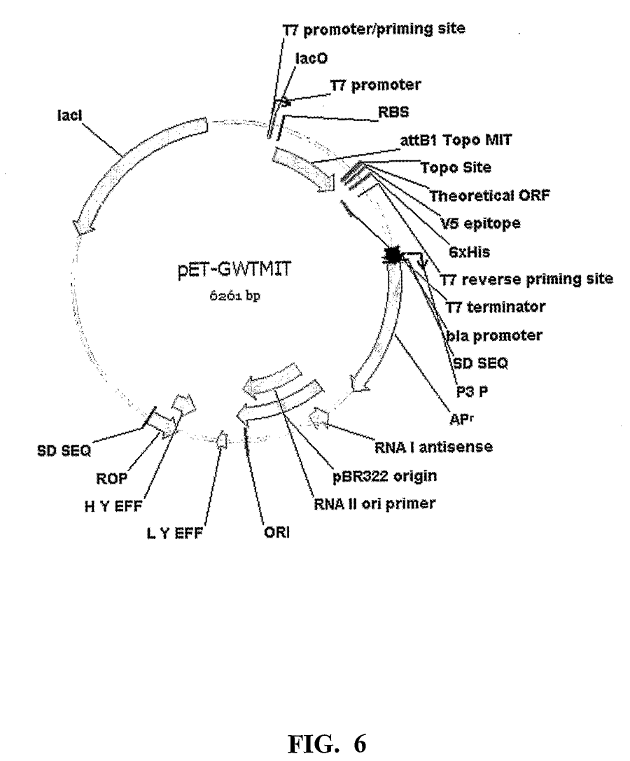

Thus, by way of specific reference to FIG. 6, and for illustration purposes only, FIG. 6 depicts a prophetic pET-GWTMIT vector nucleic acid molecule which comprises a recombination site, a topoisomerase recognition sequence and one modified intein nucleotide sequence. The vector is approximately 6.2 Kbp in size and contains, inter alia, an Sp6 promoter/priming site, a T7 promoter/priming site, and a pBR322 origin as well as an ampicillin resistance gene (which may be substituted with other selectable antibiotic resistance genes such as, for example, kanamycin, spectinomycin). The nucleotide sequence of the PET-GWTMIT vector depicted in FIG. 6 is shown in Table 4.

TABLE 4
Nucleotide Sequence of pETGWTIMIT (SEQ ID NO:10)

pET-GWTMIT
caaggagatggcgcccaacagtcccccggccacggggcctgccaccatac
ccacgccgaaacaagcgctcatgagcccgaagtggcgagcccgatcttcc
ccatcggtgatgtcggcgatataggcgccagcaaccgcacctgtggcgcc
ggtgatgccggccacgatgcgtccggcgtagaggatcgagatctcgatcc
cgcgaaattaatacgactcactataggggaattgtgagcggataacaatt
cccctctagaaataattttgtttaactttaagaaggaattcgccctcgca
gagggcactcggatcttcgatccggtcaccggtacaacgcatcgcatcga
ggatgttgtcggtgggcgcaagcctattcatgtcgtggctgctgccaagg
acggaacgctgcatgcgcggcccgtggtgtcctggttcgaccagggaacg
cgggatgtgatcgggttgcggatcgccggtggcgccatcctgtgggcgac
acccgatcacaaggtgctgacagagtacggctggcgtgccgccggggaac
tccgcaagggagacagggtggcgcaaccgcgacgcttcgatggattcggt
gacagtgcgccgattccgacaagtttgtacaaaaaagcaggcagcgcgcg
cgtgcaggcgctcgcggatgccctggatgacaaattcctgcacgacatgc
tggcggaagaactccgctattccgtgatccgagaagtgctgccaacgcgg
cgggcacgaacgttcggcctcgaggaactgcacaccctcgtcgccgaagg
ggtccttgtacacaacnnnnnnnnnnnnnnnnnnnnnnnnggtaagccta
tccctaaccctctcctcggtctcgattctacgcgtaccggtcatcatcac
catcaccattgagtttgatccggctgctaacaaagcccgaaaggaagctg
agttggctgctgccaccgctgagcaataactagcataaccccttggggcc
tctaaacgggtcttgaggggttttttgctgaaaggaggaactatatccgg
atatcccgcaagaggcccggcagtaccggcataaccaagcctatgcctac
agcatccagggtgacggtgccgaggatgacgatgagcgcattgttagatt
tcatacacggtgcctgactgcgttagcaatttaactgtgataaactaccg
cattaaagcttatcgatgataagctgtcaaacatgagaattaattcttga
agacgaaagggcctcgtgatacgcctatttttataggttaatgtcatgat
aataatggtttcttagacgtcaggtggcacttttcggggaaatgtgcgcg
gaacccctatttgtttatttttctaaatacattcaaatatgtatccgctc
atgagacaataaccctgataaatgcttcaataatattgaaaaaggaagag
tatgagtattcaacatttccgtgtcgcccttattcccttttttgcggcat
tttgccttcctgtttttgctcacccagaaacgctggtgaaagtaaaagat
gctgaagatcagttgggtgcacgagtgggttacatcgaactggatctcaa
cagcggtaagatccttgagagttttcgccccgaagaacgttttccaatga
tgagcacttttaaagttctgctatgtggcgcggtattatcccgtgttgac
gccgggcaagagcaactcggtcgccgcatacactattctcagaatgactt
ggttgagtactcaccagtcacagaaaagcatcttacggatggcatgacag
taagagaattatgcagtgctgccataaccatgagtgataacactgcggcc
aacttacttctgacaacgatcggaggaccgaaggagctaaccgctttttt
gcacaacatgggggatcatgtaactcgccttgatcgttgggaaccggagc
tgaatgaagccataccaaacgacgagcgtgacaccacgatgcctgcagca
atggcaacaacgttgcgcaaactattaactggcgaactacttactctagc
ttcccggcaacaattaatagactggatggaggcggataaagttgcaggac
cacttctgcgctcggcccttccggctggctggtttattgctgataaatct
ggagccggtgagcgtgggtctcgcggtatcattgcagcactggggccaga
tggtaagccctcccgtatcgtagttatctacacgacggggagtcaggcaa
ctatggatgaacgaaatagacagatcgctgagataggtgcctcactgatt
aagcattggtaactgtcagaccaagtttactcatatatactttagattga
tttaaaacttcatttttaatttaaaaggatctaggtgaagatcctttttg
ataatctcatgaccaaaatcccttaacgtgagttttcgttccactgagcg
tcagaccccgtagaaaagatcaaaggatcttcttgagatcctttttttct
gcgcgtaatctgctgcttgcaaacaaaaaaaccaccgctaccagcggtgg
tttgtttgccggatcaagagctaccaactctttttccgaaggtaactggc
ttcagcagagcgcagataccaaatactgtccttctagtgtagccgtagtt
aggccaccacttcaagaactctgtagcaccgcctacatacctcgctctgc
taatcctgttaccagtggctgctgccagtggcgataagtcgtgtcttacc
gggttggactcaagacgatagttaccggataaggcgcagcggtcgggctg
aacggggggttcgtgcacacagcccagcttggagcgaacgacctacaccg
aactgagatacctacagcgtgagctatgagaaagcgccacgcttcccgaa
gggagaaaggcggacaggtatccggtaagcggcagggtcggaacaggaga
gcgcacgagggagcttccagggggaaacgcctggtatctttatagtcctg
tcgggtttcgccacctctgacttgagcgtcgatttttgtgatgctcgtca
ggggggcggagcctatggaaaaacgccagcaacgcggcctttttacggtt
cctggccttttgctggccttttgctcacatgttctttcctgcgttatccc
ctgattctgtggataaccgtattaccgcctttgagtgagctgataccgct
cgccgcagccgaacgaccgagcgcagcgagtcagtgagcgaggaagcgga
agagcgcctgatgcggtattttctccttacgcatctgtgcggtatttcac
accgcaatggtgcactctcagtacaatctgctctgatgccgcatagttaa
gccagtatacactccgctatcgctacgtgactgggtcatggctgcgcccc
gacacccgccaacacccgctgacgcgccctgacgggcttgtctgctcccg
gcatccgcttacagacaagctgtgaccgtctccgggagctgcatgtgtca
gaggttttcaccgtcatcaccgaaacgcgcgaggcagctgcggtaaagct
catcagcgtggtcgtgaagcgattcacagatgtctgcctgttcatccgcg
tccagctcgttgagtttctccagaagcgttaatgtctggcttctgataaa
gcgggccatgttaagggcggttttttcctgtttggtcactgatgcctccg
tgtaagggggatttctgttcatgggggtaatgataccgatgaaacgagag
aggatgctcacgatacgggttactgatgatgaacatgcccggttactgga
acgttgtgagggtaaacaactggcggtatggatgcggcgggaccagagaa
aaatcactcagggtcaatgccagcgcttcgttaatacagatgtaggtgtt
ccacagggtagccagcagcatcctgcgatgcagatccggaacataatggt
gcagggcgctgacttccgcgtttccagactttacgaaacacggaaaccga
agaccattcatgttgttgctcaggtcgcagacgttttgcagcagcagtcg
cttcacgttcgctcgcgtatcggtgattcattctgctaaccagtaaggca
accccgccagcctagccgggtcctcaacgacaggagcacgatcatgcgca
cccgtggccaggacccaacgctgcccgagatgcgccgcgtgcggctgctg
gagatggcggacgcgatggatatgttctgccaagggttggtttgcgcatt
cacagttctccgcaagaattgattggctccaattcttggagtggtgaatc
cgttagcgaggtgccgccggcttccattcaggtcgaggtggcccggctcc
atgcaccgcgacgcaacgcggggaggcagacaaggtatagggcggcgcct
acaatccatgccaacccgttccatgtgctcgccgaggcggcataaatcgc
cgtgacgatcagcggtccaatgatcgaagttaggctggtaagagccgcga
gcgatccttgaagctgtccctgatggtcgtcatctacctgcctggacagc
atggcctgcaacgcgggcatcccgatgccgccggaagcgagaagaatcat
aatggggaaggccatccagcctcgcgtcgcgaacgccagcaagacgtagc
ccagcgcgtcggccgccatgccggcgataatggcctgcttctcgccgaaa
cgtttggtggcgggaccagtgacgaaggcttgagcgagggcgtgcaagat
tccgaataccgcaagcgacaggccgatcatcgtcgcgctccagcgaaagc
ggtcctcgccgaaaatgacccagagcgctgccggcacctgtcctacgagt
tgcatgataaagaagacagtcataagtgcggcgacgatagtcatgccccg
cgcccaccggaaggagctgactgggttgaaggctctcaagggcatcggtc
gagatcccggtgcctaatgagtgagctaacttacattaattgcgttgcgc
tcactgcccgctttccagtcgggaaacctgtcgtgccagctgcattaatg
aatcggccaacgcgcggggagaggcggtttgcgtattgggcgccagggtg
gtttttcttttcaccagtgagacgggcaacagctgattgcccttcaccgc
ctggccctgagagagttgcagcaagcggtccacgctggtttgccccagca
ggcgaaaatcctgtttgatggtggttaacggcgggatataacatgagctg
tcttcggtatcgtcgtatcccactaccgagatatccgcaccaacgcgcag
cccggactcggtaatggcgcgcattgcgcccagcgccatctgatcgttgg
caaccagcatcgcagtgggaacgatgccctcattcagcatttgcatggtt
tgttgaaaaccggacatggcactccagtcgccttcccgttccgctatcgg
ctgaatttgattgcgagtgagatatttatgccagccagccagacgcagac
gcgccgagacagaacttaatgggcccgctaacagcgcgatttgctggtga
cccaatgcgaccagatgctccacgcccagtcgcgtaccgtcttcatggga
gaaaataatactgttgatgggtgtctggtcagagacatcaagaaataacg
ccggaacattagtgcaggcagcttccacagcaatggcatcctggtcatcc
agcggatagttaatgatcagcccactgacgcgttgcgcgagaagattgtg
caccgccgctttacaggcttcgacgccgcttcgttctaccatcgacacca
ccacgctggcacccagttgatcggcgcgagatttaatcgccgcgacaatt
tgcgacggcgcgtgcagggccagactggaggtggcaacgccaatcagcaa
cgactgtttgcccgccagttgttgtgccacgcggttgggaatgtaattca
gctccgccatcgccgcttccactttttcccgcgttttcgcagaaacgtgg
ctggcctggttcaccacgcgggaaacggtctgataagagacaccggcata
ctctgcgacatcgtataacgttactggtttcacattcaccaccctgaatt
gactctcttccgggcgctatcatgccataccgcgaaaggttttgcgccat
tcgatggtgtccgggatctcgacgctctcccttatgcgactcctgcatta
ggaagcagcccagtagtaggttgaggccgttgagcaccgccgccgcaagg
aatggtgcatg

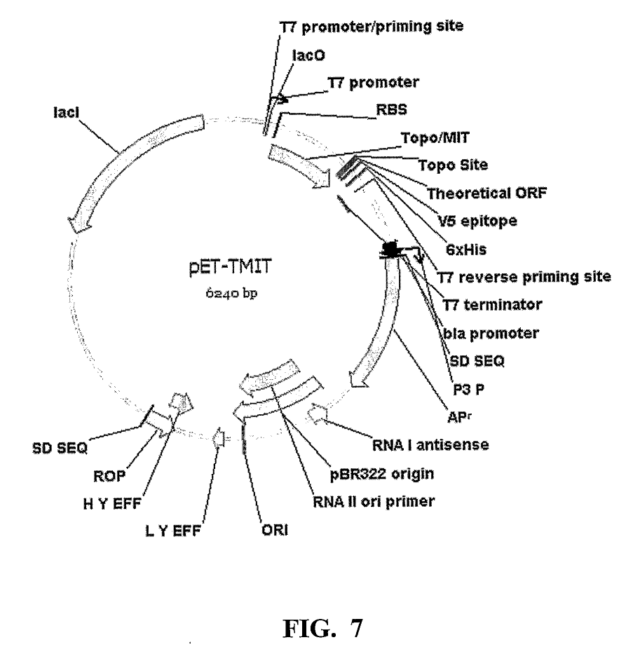

Thus, by way of specific reference to FIG. 7, and for illustration purposes only, FIG. 7 depicts a prophetic pET-TMIT vector nucleic acid molecule. which comprises a topoisomerase recognition sequence and one modified intein nucleotide sequence. The vector is approximately 6.24 Kbp in size and contains, inter alia, an Sp6 promoter/priming site, a T7 promoter/priming site, and a pBR322 origin as well as an ampicillin resistance gene (which may be substituted with other selectable antibiotic resistance genes such as, for example, kanamycin, spectinomycin). The nucleotide sequence of the pET-TMIT vector depicted in FIG. 7 is shown in Table 5.

TABLE 5
Nucleotide Sequence of pET-TMIT (SEQ ID NO:11)

pET-TMIT
caaggagatggcgcccaacagtcccccggccacggggcctgccaccatac
ccacgccgaaacaagcgctcatgagcccgaagtggcgagcccgatcttcc
ccatcggtgatgtcggcgatataggcgccagcaaccgcacctgtggcgcc
ggtgatgccggccacgatgcgtccggcgtagaggatcgagatctcgatcc
cgcgaaattaatacgactcactataggggaattgtgagcggataacaatt
cccctctagaaataattttgtttaactttaagaaggaattcgccctcgca
gagggcactcggatcttcgatccggtcaccggtacaacgcatcgcatcga
ggatgttgtcggtgggcgcaagcctattcatgtcgtggctgctgccaagg
acggaacgctgcatgcgcggcccgtggtgtcctggttcgaccagggaacg
cgggatgtgatcgggttgcggatcgccggtggcgccatcctgtgggcgac
acccgatcacaaggtgctgacagagtacggctggcgtgccgccggggaac
tccgcaagggagacagggtggcgcaaccgcgacgcttcgatggattcggt
gacagtgcgccgattccggcgcgcgtgcaggcgctcgcggatgccctgga
tgacaaattcctgcacgacatgctggcggaagaactccgctattccgtga
tccgagaagtgctgccaacgcggcgggcacggacgttcggcctcgaggtc
gaggaactgcacaccctcgtcgccgaaggggtccttgtacacaacnnnnn
nnnnnnnnnnnnnnnnnnnggtaagcctatccctaaccctctcctcggtc
tcgattctacgcgtaccggtcatcatcaccatcaccattgagtttgatcc
ggctgctaacaaagcccgaaaggaagctgagttggctgctgccaccgctg
agcaataactagcataaccccttggggcctctaaacgggtcttgaggggt
tttttgctgaaaggaggaactatatccggatatcccgcaagaggcccggc
agtaccggcataaccaagcctatgcctacagcatccagggtgacggtgcc
gaggatgacgatgagcgcattgttagatttcatacacggtgcctgactgc
gttagcaatttaactgtgataaactaccgcattaaagcttatcgatgata
agctgtcaaacatgagaattaattcttgaagacgaaagggcctcgtgata
cgcctatttttataggttaatgtcatgataataatggtttcttagacgtc
aggtggcacttttcggggaaatgtgcgcggaacccctatttgtttatttt
tctaaatacattcaaatatgtatccgctcatgagacaataaccctgataa
atgcttcaataatattgaaaaaggaagagtatgagtattcaacatttccg
tgtcgcccttattcccttttttgcggcattttgccttcctgtttttgctc
acccagaaacgctggtgaaagtaaaagatgctgaagatcagttgggtgca
cgagtgggttacatcgaactggatctcaacagcggtaagatccttgagag
ttttcgccccgaagaacgttttccaatgatgagcacttttaaagttctgc
tatgtggcgcggtattatcccgtgttgacgccgggcaagagcaactcggt
cgccgcatacactattctcagaatgacttggttgagtactcaccagtcac
agaaaagcatcttacggatggcatgacagtaagagaattatgcagtgctg
ccataaccatgagtgataacactgcggccaacttacttctgacaacgatc
ggaggaccgaaggagctaaccgcttttttgcacaacatgggggatcatgt
aactcgccttgatcgttgggaaccggagctgaatgaagccataccaaacg
acgagcgtgacaccacgatgcctgcagcaatggcaacaacgttgcgcaaa
ctattaactggcgaactacttactctagcttcccggcaacaattaataga
ctggatggaggcggataaagttgcaggaccacttctgcgctcggcccttc
cggctggctggtttattgctgataaatctggagccggtgagcgtgggtct
cgcggtatcattgcagcactggggccagatggtaagccctcccgtatcgt
agttatctacacgacggggagtcaggcaactatggatgaacgaaatagac
agatcgctgagataggtgcctcactgattaagcattggtaactgtcagac
caagtttactcatatatactttagattgatttaaaacttcatttttaatt
taaaaggatctaggtgaagatcctttttgataatctcatgaccaaaatcc
cttaacgtgagttttcgttccactgagcgtcagaccccgtagaaaagatc
aaaggatcttcttgagatcctttttttctgcgcgtaatctgctgcttgca
aacaaaaaaaccaccgctaccagcggtggtttgtttgccggatcaagagc
taccaactctttttccgaaggtaactggcttcagcagagcgcagatacca
aatactgtccttctagtgtagccgtagttaggccaccacttcaagaactc
tgtagcaccgcctacatacctcgctctgctaatcctgttaccagtggctg
ctgccagtggcgataagtcgtgtcttaccgggttggactcaagacgatag
ttaccggataaggcgcagcggtcgggctgaacggggggttcgtgcacaca
gcccagcttggagcgaacgacctacaccgaactgagatacctacagcgtg
agctatgagaaagcgccacgcttcccgaagggagaaaggcggacaggtat
ccggtaagcggcagggtcggaacaggagagcgcacgagggagcttccagg
gggaaacgcctggtatctttatagtcctgtcgggtttcgccacctctgac
ttgagcgtcgatttttgtgatgctcgtcaggggggcggagcctatggaaa
aacgccagcaacgcggcctttttacggttcctggccttttgctggccttt
tgctcacatgttctttcctgcgttatcccctgattctgtggataaccgta
ttaccgcctttgagtgagctgataccgctcgccgcagccgaacgaccgag
cgcagcgagtcagtgagcgaggaagcggaagagcgcctgatgcggtattt
tctccttacgcatctgtgcggtatttcacaccgcaatggtgcactctcag
tacaatctgctctgatgccgcatagttaagccagtatacactccgctatc
gctacgtgactgggtcatggctgcgccccgacacccgccaacacccgctg
acgcgccctgacgggcttgtctgctcccggcatccgcttacagacaagct
gtgaccgtctccgggagctgcatgtgtcagaggttttcaccgtcatcacc
gaaacgcgcgaggcagctgcggtaaagctcatcagcgtggtcgtgaagcg
attcacagatgtctgcctgttcatccgcgtccagctcgttgagtttctcc
agaagcgttaatgtctggcttctgataaagcgggccatgttaagggcggt
tttttcctgtttggtcactgatgcctccgtgtaagggggatttctgttca
tgggggtaatgataccgatgaaacgagagaggatgctcacgatacgggtt
actgatgatgaacatgcccggttactggaacgttgtgagggtaaacaact
ggcggtatggatgcggcgggaccagagaaaaatcactcagggtcaatgcc
agcgcttcgttaatacagatgtaggtgttccacagggtagccagcagcat
cctgcgatgcagatccggaacataatggtgcagggcgctgacttccgcgt
ttccagactttacgaaacacggaaaccgaagaccattcatgttgttgctc
aggtcgcagacgttttgcagcagcagtcgcttcacgttcgctcgcgtatc
ggtgattcattctgctaaccagtaaggcaaccccgccagcctagccgggt
cctcaacgacaggagcacgatcatgcgcacccgtggccaggacccaacgc
tgcccgagatgcgccgcgtgcggctgctggagatggcggacgcgatggat
atgttctgccaagggttggtttgcgcattcacagttctccgcaagaattg
attggctccaattcttggagtggtgaatccgttagcgaggtgccgccggc
ttccattcaggtcgaggtggcccggctccatgcaccgcgacgcaacgcgg
ggaggcagacaaggtatagggcggcgcctacaatccatgccaacccgttc
catgtgctcgccgaggcggcataaatcgccgtgacgatcagcggtccaat
gatcgaagttaggctggtaagagccgcgagcgatccttgaagctgtccct
gatggtcgtcatctacctgcctggacagcatggcctgcaacgcgggcatc
ccgatgccgccggaagcgagaagaatcataatggggaaggccatccagcc
tcgcgtcgcgaacgccagcaagacgtagcccagcgcgtcggccgccatgc
cggcgataatggcctgcttctcgccgaaacgtttggtggcgggaccagtg
acgaaggcttgagcgagggcgtgcaagattccgaataccgcaagcgacag
gccgatcatcgtcgcgctccagcgaaagcggtcctcgccgaaaatgaccc
agagcgctgccggcacctgtcctacgagttgcatgataaagaagacagtc
ataagtgcggcgacgatagtcatgccccgcgcccaccggaaggagctgac
tgggttgaaggctctcaagggcatcggtcgagatcccggtgcctaatgag
tgagctaacttacattaattgcgttgcgctcactgcccgctttccagtcg
ggaaacctgtcgtgccagctgcattaatgaatcggccaacgcgcggggag
aggcggtttgcgtattgggcgccagggtggtttttcttttcaccagtgag
acgggcaacagctgattgcccttcaccgcctggccctgagagagttgcag
caagcggtccacgctggtttgccccagcaggcgaaaatcctgtttgatgg
tggttaacggcgggatataacatgagctgtcttcggtatcgtcgtatccc
actaccgagatatccgcaccaacgcgcagcccggactcggtaatggcgcg
cattgcgcccagcgccatctgatcgttggcaaccagcatcgcagtgggaa
cgatgccctcattcagcatttgcatggtttgttgaaaaccggacatggca
ctccagtcgccttcccgttccgctatcggctgaatttgattgcgagtgag
atatttatgccagccagccagacgcagacgcgccgagacagaacttaatg
ggcccgctaacagcgcgatttgctggtgacccaatgcgaccagatgctcc
acgcccagtcgcgtaccgtcttcatgggagaaaataatactgttgatggg
tgtctggtcagagacatcaagaaataacgccggaacattagtgcaggcag
cttccacagcaatggcatcctggtcatccagcggatagttaatgatcagc
ccactgacgcgttgcgcgagaagattgtgcaccgccgctttacaggcttc
gacgccgcttcgttctaccatcgacaccaccacgctggcacccagttgat
cggcgcgagatttaatcgccgcgacaatttgcgacggcgcgtgcagggcc
agactggaggtggcaacgccaatcagcaacgactgtttgcccgccagttg
ttgtgccacgcggttgggaatgtaattcagctccgccatcgccgcttcca
ctttttcccgcgttttcgcagaaacgtggctggcctggttcaccacgcgg
gaaacggtctgataagagacaccggcatactctgcgacatcgtataacgt
tactggtttcacattcaccaccctgaattgactctcttccgggcgctatc
atgccataccgcgaaaggttttgcgccattcgatggtgtccgggatctcg
acgctctcccttatgcgactcctgcattaggaagcagcccagtagtaggt
tgaggccgttgagcaccgccgccgcaaggaatggtgcatg

Positive Selection Vectors for Use with the Compositions of the Invention

In certain embodiments of the present invention, the at least one modified intein nucleotide sequence or a functional derivative or homolog thereof with the one or more sequence tags may be used in conjunction with a positive selection marker in both conventional and recombination-based cloning and expression systems. Preferably, selectable markers used in the methods described above are positive selection markers (e.g., antibiotic resistance markers such as ampicillin, tetracycline, kanamycin, neomycin. and G-418 resistance markers). As defined herein, selecting for a nucleic acid molecule includes (a) selecting or enriching for the presence of the desired nucleic acid molecule (referred to as a “positive selection scheme”), and (b) selecting or enriching against the presence of nucleic acid molecules that are not the desired nucleic acid molecule (referred to as a “negative selection scheme”).

In one particular embodiment of a modified intein nucleotide sequence or a functional derivative or homolog thereof-containing vector (e.g., donor, entry, destination or expression vectors), the vector (e.g., donor, entry, destination or expression vectors) nucleic acid molecules of the invention may further comprise a positive selection nucleotide sequence operatively linked to a suitable promotor/operator region and further comprising a multiple cloning site with one or more unique restriction enzyme recognition sites, together with one or more origins of replication, and one or more selectable markers such as, for example, and not by way of limitation, an ampicillin resistance gene.

In another particular embodiment of a modified intein nucleotide sequence or a functional derivative or homolog thereof-containing vector, the vector nucleic acid molecules of the invention may further comprise a positive selection nucleotide sequence fusion (e.g., C-terminal/N-terminal fusions) construct or a functional derivative or homolog thereof operatively linked to the positive selection marker promotor/operator region, together with one or more origins of replication, one or more cloning sites with one or more unique restriction enzyme recognition sites and one or more selectable markers, such as, for example, and not by way of limitation, an ampicillin resistance gene.

In each of these two afore-mentioned embodiments, the presence of the positive selection marker (e.g., antibiotic resistance markers such as ampicillin, tetracycline, kanamycin, neomycin. and G-418 resistance markers) is serving as a positive selection scheme in that insertion of a nucleotide sequence of interest in the multiple cloning site of the vector (e.g., donor, entry, destination or expression vectors) nucleic acid molecules generates a recombinant vector molecule with a functional positive selection gene. As a result, transformation of recipient bacterial host cells with ligation products results in recombinant bacterial clones which will have the appropriate phenotype of antibiotic resistance conferred upon them by the antibiotic resistance gene. In this case, however, as there is no means to negatively select for non-recombinants, the linearized parental vector (e.g., donor, entry, destination or expression vectors) often will need to be treated (for example, by dephosphorylation) so as to prevent unwanted parental vector (e.g., donor, entry, destination or expression vectors) self-ligation. Subsequent expression of the protein of interest, followed by the self-splicing reaction of the modified intein under suitable conditions of pH and temperature serves to excise the modified intein with the one or more sequence tags thereby facilitating recovery of the protein of interest.

It is of course possible to make use of both the positive selection scheme based upon the a positive selection marker (e.g., antibiotic resistance markers such as ampicillin, tetracycline, kanamycin, neomycin. and G-418 resistance markers) in conjunction with the negative selection scheme based upon a negative selection marker (e.g., ccdB). In this embodiment of a modified intein nucleotide sequence or a functional derivative or homolog thereof-containing vector (e.g., donor, entry, destination or expression vectors), the nucleotide sequence of interest in a first population to be cloned may further comprise, or be fused with, a positive selection marker nucleotide sequence operatively linked to a suitable promotor/operator region wherein the nucleotide sequence of interest and the positive selection marker nucleotide sequence further comprise two recombination sites and/or one or more topoisomerase sites and their corresponding topoisomerase proteins. This nucleotide sequence of interest and the positive selection marker nucleotide sequence is then transferred via recombination to a nucleic acid molecule comprising a nucleotide sequence encoding a negative selection marker (e.g., ccdB) further comprising two recombination sites and/or one or more topoisomerase recognition sequences and their corresponding topoisomerase proteins.

In this particular embodiment, by conducting a recombination reaction such that all or a portion of the nucleic acid molecules of interest further comprising, or fused with, the positive selection marker nucleotide sequence with recombination sites and/or one or more topoisomerase recognition sequences and their corresponding topoisomerase proteins in a first population is recombined with one or more molecules from the donor or expression vector product nucleic acid molecules, a third population of hybrid nucleic acid molecules is formed. Through resolution of the resultant cointegrates, the nucleic acid molecule of interest with the positive selection marker nucleotide sequence is cloned. Thus, in this embodiment, a negative selection marker (e.g., ccdB) and a positive selection marker-mediated double selection scheme is provided. The presence of the lethal ccdB gene on the parental donor vector serves as a negative selection marker in that transformation of bacterial cells susceptible to the lethal effects of the ccdB toxin with non-recombinant parental donor vector molecules, cointegrates, or donor vector byproduct molecules results in cell death.

In this embodiment, only true recombinant plasmid vector clones containing the nucleic acid of interest containing the at least one modified intein nucleotide sequence or a functional derivative or homolog thereof may be used in conjunction with a positive selection marker nucleotide sequence, but without the ccdB nucleotide sequence, will be capable of growth as a result of the recombinase-mediated excision of the ccdB gene. The presence of the antibiotic resistance gene serves as a positive selection marker in that transformation of recipient bacterial host cells with recombination products results in recombinant bacterial clones which will have the appropriate phenotype of antibiotic resistance conferred upon them by the presence of the appropriate antibiotic resistance gene. Cleavage of the at least one one modified intein protein sequence under the appropriate conditions (e.g., temperature and pH) serves to release the sequence tag (e.g., an affinity tag) from the protein of interest.

Methods of Cloning and Expression Using the Compositions of the Invention

In another aspect, the invention relates to a method of cloning comprising: (a) obtaining at least one nucleic acid molecule of interest to be cloned comprising one or more recombination sites; and (b) transferring all or a portion of said molecule into one or more vectors comprising a nucleotide sequence encoding at least one modified intein or a functional derivative or homolog thereof located between one or more recombination sites. In particular embodiments, the nucleotide sequence encoding the at least one modified intein or a functional derivative or homolog thereof with one or more sequence tags will be removed from the protein of interest by intein-mediated cleavage following expression of the protein of interest. Expression of the protein of interest, followed by cleavage of the at least one one modified intein protein sequence under the appropriate conditions (e.g., temperature and pH) serves to release the sequence tag (e.g., an affinity tag) from the protein of interest.

The invention further includes vectors prepared by such methods, compositions comprising these vectors, and methods using these vectors.

In another aspect, the invention relates to a method of cloning comprising: (a) obtaining at least one nucleic acid molecule of interest to be cloned comprising one or more recombination sites and/or one or more topoisomerase recognition sites and/or one or more topoisomerases; and (b) transferring all or a portion of said molecule into one or more vectors comprising a nucleotide sequence encoding at least one modified intein or a functional derivative or homolog thereof located between one or more recombination sites and/or one or more topoisomerase recognition sites, together with the one or more sequence tags. In particular embodiments, the nucleotide sequence encoding at least one modified intein or a functional derivative or homolog thereof will be removed from the expressed protein by intein mediated cleavage following expression of the protein of interest. Expression of the protein of interest, followed by cleavage of the at least one one modified intein protein sequence under the appropriate conditions (e.g., temperature and pH) serves to release the sequence tag (e.g., an affinity tag) from the protein of interest.

The invention further includes vectors prepared by such methods, compositions comprising these vectors, and methods using these vectors.

Such vectors generated by this method or variations thereof will often comprise, in addition to the nucleotide sequence encoding at least one modified intein or a functional derivative or homolog thereof, one or more recombination sites and/or one or more topoisomerase recognition sites and/or one or more topoisomerases, together with the one or more sequence tags, and the transfer of the nucleic acid molecules of interest into such vectors is preferably accomplished by recombination between one or more sites on the vectors and one or more sites on the molecules of the invention. In another aspect, the product molecules of the invention may be converted to molecules which function as vectors by including the necessary vector sequences (e.g., origins of replication). Thus, according to the invention, such vectors sequences may be incorporated into the product molecules through the use of starting vector donor molecules containing such sequences. Such vector sequences may be added at one or a number of desired locations in the product molecules, depending on the location of the sequence within the starting molecule and the order of addition of the starting molecules in the product molecule. The product molecule containing the vector sequences may be in linear form or may be converted to a circular or supercoiled form by causing recombination of recombination sites within the product molecule or by a topoisomerase-mediated joining reaction. Circularization of such product molecule maybe accomplished by recombining recombination sites at or near both termini of the product molecule.

The vector sequences generated by the methods of the present invention may comprise one or a number of elements and/or functional sequences and/or sites (or combinations thereof) including one or more sequencing or amplification primer sites, one or more multiple cloning sites, one or more selectable markers (e.g., toxic genes, antibiotic resistance genes, selectable markers etc.), one or more transcription or translation sites or signals, one or more transcription or translation termination sites, one or more topoisomerase recognition sites, one or more topoisomerases, one or more origins of replication, one or more recombination sites (or portions thereof), etc. The vector sequences used in the invention may also comprise stop codons which may be suppressed to allow expression of desired fusion proteins as described herein. Thus, according to the invention, vector sequences may be used to introduce one or more of such elements, functional sequences and/or sites into any of the nucleic acid molecule of the invention, and such sequences may be used to further manipulate or analyze any such nucleic acid molecule cloned into such vectors. For example, primer sites provided by a vector (preferably located on both sides of the insert cloned in such vector) allow sequencing or amplification of all or a portion of a product molecule cloned into the vector. Additionally, transcriptional or regulatory sequences contained by the vector allows expression of peptides, polypeptides or proteins encoded by all or a portion of the product molecules cloned to the vector.

In addition to the nucleotide sequence encoding at least one modified intein, the nucleotide sequence encoding the protein of interest may also be modified to contain a gene, portion of genes or sequence tags (such as GUS, GST, GFP, His tags, epitope tags and the like) provided by the vectors to allow creation of populations of gene fusions with the product molecules cloned in the vector or allows production of a number of peptide, polypeptide or protein fusions encoded by the sequence tags provided by the vector in combination with the product sequences cloned in such vector. Such genes, portions of genes or sequence tags may be used in combination with optionally suppressed stop codons to allow controlled expression of fusion proteins encoded by the sequence of interest being cloned into the vector and the vector supplied gene or tag sequence. In a construct, the vector may comprise one or more recombination sites, one or more stop codons and one or more tag sequences. In some embodiments, the tag sequences may be adjacent to a recombination site. Optionally, a stop codon may be incorporated into the sequence of the tag or in the sequence of the recombination site in order to allow controlled addition of the tag sequence to the gene of interest. In any event, it is intended herein that such a gene, portion of genes or sequence tag(s) will not in any way affect the ability of the at least one modified intein protein sequence to be released via intein-mediated cleavage following expression of the protein of interest.

In embodiments of this type, the gene of interest may be inserted into the vector by recombinational cloning such that the tag and the coding sequence of the gene of interest are in the same reading frame. The gene of interest may be provided with translation initiation signals, e.g., Shine-Delgarno sequences, Kozak sequences and/or IRES sequences, in order to permit the expression of the gene with a native N-terminal when the stop codon is not suppressed. The gene of interest may also be provided with a stop codon at the 3′-end of the coding sequence. In some embodiments, a tag sequence may be provided at both the N- and C-terminals of the gene of interest. Optionally, the tag sequence at the N-terminal may be provided with a stop codon and the gene of interest may be provided with a stop codon and the tag at the C-terminal may be provided with a stop codon. The stop codons may be the same or different. In some embodiments, the stop codon of the N-terminal tag is different from the stop codon of the gene of interest. In embodiments of this type, suppressor tRNAs corresponding to one or both of the stop codons may be provided. When both are provided, each of the suppressor tRNAs may independently be provided on the same vector, a different vector or in the host cell genome. The suppressor tRNA need not both be provided in the same way, for example, one may be provided on the vector containing the gene of interest while the other may be provided in the host cell genome. In this way, the nucleic acid molecules of one such aspect of the invention may comprise a suppressible stop codon that separates two coding regions. Depending on the location of the expression signals (e.g., promoters), expression of the suppressor Trna results in suppression of the stop codon(s), thereby allowing the production of a fusion peptide, for example a fusion peptide having an affinity tag sequence at the N- and/or C-terminus of the expressed protein. By not suppressing the stop codon(s), expression of the sequence of interest without the N- and/or C-terminal tag sequence may be accomplished. Thus, the invention allows through recombination efficient construction of vectors containing a gene or sequence of interest (e.g., one or more open reading frames or “orfs”) for controlled expression of fusion proteins depending on the need. Preferably, the starting nucleic acid molecules or product molecules of the invention that are cloned into one or more vectors comprise at least one open reading frame (orf). Such starting or product molecules may also comprise functional sequences (e.g., primer sites, transcriptional or translation sites or signals, termination sites (e.g., stop codons which may be optionally suppressed), origins of replication, and the like) and preferably comprises sequences that regulate gene expression including transcriptional regulatory sequences and sequences that function as internal ribosome entry sites (IRES). Preferably, at least one of the starting or product molecules and/or vectors comprise sequences that function as a promoter. Such starting or product molecules and/or vectors may also comprise transcription termination sequences, selectable markers, restriction enzyme recognition sites, and the like.

In some embodiments of the present invention, the vectors generated by the methods of the present invention comprise two copies of the same selectable marker, each copy flanked by recombination sites and/or topoisomerase recognition sites. In other embodiments, the vector comprises two different selectable markers each flanked by two recombination sites. In some embodiments, one or more of the selectable markers may be a negative selectable marker (e.g., ccdB).

The nucleic acid molecules to be joined by the methods of the invention (e.g., the “starting molecules”) may be used to produce one or more hybrid molecules containing all or a portion of the starting molecules (e.g., the “product nucleic acid molecules”). The starting molecules can be any nucleic acid molecule derived from any source or produced by any method. Such molecules may be derived from natural sources (such as cells, tissue, and organs from any animal or non-animal source) or may be non-natural (e.g., derivative nucleic acids) or synthetically derived. The segments or molecules for use in the invention may be produced by any means known to those skilled in the art including, but not limited to, amplification such as by PCR, isolation from natural sources, chemical synthesis, shearing or restriction digest of larger nucleic acid molecules (such as genomic or cDNA), transcription, reverse transcription and the like, and nucleotide sequences encoding modified inteins, recombination sites and/or topoisomerase recognition sites and/or topoisomerases may be added to such molecules by any means known to those skilled in the art including ligation of adapters containing nucleotide sequences encoding modified inteins, recombination sites and/or topoisomerase recognition sites and/or topoisomerases, amplification or nucleic acid synthesis using primers containing nucleotide sequences encoding modified inteins, recombination sites and/or topoisomerase recognition sites and/or topoisomerases, insertion or integration of nucleic acid molecules (e.g., transposons or integration sequences) containing nucleotide sequences encoding modified inteins, recombination sites and/or topoisomerase recognition sites and/or topoisomerases, etc. In one aspect, the nucleic acid molecules used in the invention are populations of molecules such as nucleic acid libraries or cDNA libraries.

Once nucleic acid molecules are joined by recombination using methods such as those described herein, these nucleic acid molecules may then be joined to other nucleic acid molecules using standard recombinant DNA technology cloning methods or preferably by recombination-mediated joining methods and/or topoisomerase-mediated joining methods as described in detail in the afore-mentioned issued patents and pending applications.

Additional Applications for the Compositions and Methods of the Invention

Any of the starting or vector product molecules comprising the nucleotide sequence encoding at least one modified intein or a functional derivative or homolog thereof comprising one or more sequence tags and one or more topoisomerase recognition sequences and the corresponding topoisomerase proteins and/or one or more recombination sites and the corresponding recombination proteins systems of the invention may be further manipulated, analyzed or used in any number of standard molecular biology techniques or combinations of such techniques (in vitro or in vivo). These techniques include amplification, nucleic acid synthesis, protein or peptide expression (for example, fusion protein expression, antibody expression, hormone expression etc.), protein-protein interactions (2-hybrid or reverse 2-hybrid analysis), homologous recombination or gene targeting, and combinatorial library analysis and manipulation. The invention also relates to cloning the nucleic acid molecules comprising the nucleotide sequence encoding at least one modified intein or a functional derivative or homolog thereof comprising one or more sequence tags and one or more topoisomerase recognition sequences and the corresponding topoisomerase proteins and/or one or more recombination sites and the corresponding recombination proteins systems of the invention by standard cloning methodologies or by recombination into one or more vectors or converting the nucleic acid molecules of the invention into a vector by the addition of certain functional vector sequences (e.g., origins of replication). In one aspect, recombination and/or topoisomerase-mediated joining is accomplished in vitro and further manipulation or analysis is performed directly in vitro. Thus, further analysis and manipulation will not be constrained by the ability to introduce the molecules of the invention into a host cell and/or maintained in a host cell. Thus, less time and higher throughput may be accomplished by further manipulating or analyzing the molecules of the invention directly in in vitro, although in vitro analysis or manipulation can be done after passage through host cells or can be done directly in vivo (while in the host cells).

Nucleic acid synthesis steps, according to the invention, may comprise:

(a) mixing a nucleic acid molecule of interest or template with one or more primers and one or more nucleotides to form a mixture; and

(b) incubating said mixture under conditions sufficient to synthesize a nucleic acid molecule complementary to all or a portion of said molecule or template.

The synthesized molecule may then be used as a template for further synthesis of a nucleic acid molecule complementary to all or a portion of the first synthesized molecule. Accordingly, a double stranded nucleic acid molecule (e.g., DNA) may be prepared. Preferably, such second synthesis step is preformed in the presence of one or more primers and one or more nucleotides under conditions sufficient to synthesize the second nucleic acid molecule complementary to all or a portion of the first nucleic acid molecule. Typically, synthesis of one or more nucleic acid molecules is performed in the presence of one or more polymerases (preferably DNA polymerases which may be thermostable or mesophilic), although reverse transcriptases may also be used in such synthesis reactions. Accordingly, the nucleic acid molecules used as templates for the synthesis of additional nucleic acid molecules may be RNA, mRNA, DNA or non-natural or derivative nucleic acid molecules. Nucleic acid synthesis, according to the invention, may be facilitated by incorporating one or more primer sites into the product molecules through the use of starting nucleic acid molecules containing such primer sites. Thus, by the methods of the invention, primer sites may be added at one or a number of desired locations in the product molecules, depending on the location of the primer site within the starting molecule and the order of addition of the starting molecule in the product molecule. Thus, by the methods of the invention, one may add nucleotide sequences encoding at least one modified intein or a functional derivative or homolog thereof, topoisomerase recognition sequences and/or recombination sites, as well as restriction sites to molecules to be cloned.

Protein expression steps, according to the invention, may comprise:

(a) obtaining a nucleic acid molecule to be expressed which comprises one or more expression signals; and

(b) expressing all or a portion of the nucleic acid molecule under control of said expression signal thereby producing a peptide or protein encoded by said molecule or portion thereof. Representative examples of nucleic acid molecules which can be used in such protein expression steps include those nucleic acid molecules described above, as well as those nucleic acid molecules described elsewhere herein.

In this context, the expression signal may be said to be operably linked to the sequence to be expressed. The protein or peptide expressed is preferably expressed in a host cell (in vivo), although expression may be conducted in vitro using techniques well known in the art. Upon expression of the protein or peptide, the protein or peptide product may optionally be isolated or purified. Expression of the protein of interest, followed by the cleavage of the at least one one modified intein protein sequence under the appropriate conditions (e.g., temperature and pH) serves to release modified intein together with the one or more sequence tags (e.g., an affinity tag) thereby facilitating recovery of the protein of interest without any contaminating modified intein and/or sequence tags (e.g., affinity tags).

Moreover, the expressed protein or peptide may be used in various protein analysis techniques including 2-hybrid interaction, protein functional analysis and agonist/antagonist-protein interactions (e.g., stimulation or inhibition of protein function through drugs, compounds or other peptides). The novel and unique hybrid proteins or peptides (e.g., fusion proteins) produced by the invention and particularly from expression of the combinatorial molecules of the invention may generally be useful for therapeutics. Protein expression, according to the invention, may be facilitated by incorporating one or more transcription or translation signals or regulatory sequences, start codons, termination signals, splice donor/acceptor sequences (e.g., intronic sequences) and the like into the product molecules through the use of starting nucleic acid molecules containing such sequences. Thus, by the methods of the invention, expression sequences may be added at one or a number of desired locations in the product molecules, depending on the location of such sequences within the starting molecule and the order of addition of the starting molecule in the product molecule.

The invention also relates to a method of expressing one or more proteins (e.g., fusion proteins) (e.g., one, two, three, four, five, seven, ten, twelve, fifteen, twenty, thirty, fifty, etc.) comprising:

(a) obtaining at least a first nucleic acid molecule comprising at least one recombination site (or mutants, fragments, variants or derivatives thereof) (preferably the recombination site is located at or near a terminus or termini of said first nucleic acid molecule) and/or topoisomerase sites (or mutants, fragments, variants or derivatives thereof) flanking the nucleotide sequence encoding at least one modified intein or a derivative thereof and a second nucleic acid molecule comprising at least one recombination site (which is preferably located at or near a terminus or termini of said second nucleic acid molecule);

(b) causing said at least first and second nucleic acid molecules to recombine through recombination of said recombination sites, thereby producing a third nucleic acid molecule comprising all or a portion of said at least first and second molecules; and

(c) expressing one or more peptides or proteins (e.g., one, two, three, four, five, seven, ten, twelve, fifteen, twenty, thirty, fifty, etc.) encoded by said third nucleic acid molecule. In certain such embodiments, at least part of the expressed fusion protein will be encoded by the third nucleic acid molecule and at least another part will be encoded by at least part of the first and/or second nucleic acid molecules. Such a fusion protein may be produced by translation of nucleic acid which corresponds to recombination sites located between the first and second nucleic acid molecules. Thus, fusion proteins may be expressed by “reading through” mRNA corresponding to recombination sites used to connect two or more nucleic acid segments. The invention further includes fusion proteins produced by methods of the invention and mRNA which encodes such fusion proteins. Expression of the protein of interest, followed by the cleavage of the at least one one modified intein protein sequence under the appropriate conditions (e.g., temperature and pH) serves to release modified intein together with the one or more sequence tags (e.g., an affinity tag) thereby facilitating recovery of the protein of interest without any contaminating modified intein and/or sequence tags (e.g., affinity tags).

The present invention also provides methods for cloning the starting or product nucleic acid molecules of the invention into one or more vectors or converting the product molecules of the invention into one or more vectors. In one aspect, the starting molecules are recombined to make one or more product molecules and such product molecules are cloned (e.g., by conventional cloning, recombination, etc.) into one or more vectors. In another aspect, the starting molecules are cloned directly into one or more vectors such that a number of starting molecules are joined within the vector, thus creating a vector containing the product molecules of the invention. In another aspect, the starting molecules are cloned directly into one or more vectors such that the starting molecules are not joined within the vector (i.e., the starting molecules are separated by vector sequences). In yet another aspect, a combination of product molecules and starting molecules may be cloned in any order into one or more vectors, thus creating a vector comprising a new product molecule resulting from a combination of the original starting and product molecules.

In one general aspect, the invention relates to methods for inserting one or more nucleic acid molecules into one or more other nucleic acid molecules, methods for transferring one or more nucleic acid molecules which reside in a first nucleic acid molecule into a second nucleic acid molecule, and novel selection and/or screening methods based upon the nucleotide sequence encoding at least one modified intein or a functional derivative or homolog thereof for identifying nucleic acids of interest. In many embodiments, methods of the invention involve the use and/or transfer of populations of nucleic acid molecules. The invention further relates to populations of nucleic acid molecules prepared by methods of the invention and individual nucleic acid molecules prepared and/or isolated by methods of the invention.

In addition, the invention relates, in part, to methods and compositions for the identification and/or isolation of one or more populations or subpopulations of nucleic acid molecules. In specific embodiments, methods and compositions of the invention employ recombinational cloning systems, such as the Gateway® Cloning System described in detail in U.S. Pat. No. 5,888,732; PCT Publication No. WO 00/52027; U.S. application Ser. No. 09/177,387, filed Oct. 23, 1998; U.S. application Ser. No. 09/438,358, filed Nov. 12, 1999; U.S. application Ser. No. 09/517,466, filed Mar. 2, 2000, and U.S. application Ser. No. 09/732,914, filed Dec. 11, 2000 (the disclosures of all of which are incorporated herein by reference in their entireties).

Kits for Use with the Compositions and Methods of the Invention

The invention also provides kits which may be used in producing nucleic acid molecules, polypeptides, vectors, host cells, and antibodies of the invention. The invention further provides kits which may be used for the insertion of nucleic acid molecules into target nucleic acid molecules (e.g. vectors), for the transfer of nucleic acid molecules between target nucleic acid molecules, and in selection methods (e.g., sequential selection methods) of the invention.

Kits according to this aspect of the invention may comprise one or more containers, which may contain one or more of the nucleic acid molecules, primers, polypeptides, vectors, host cells, or antibodies of the invention. In particular, kits of the invention may comprise one or more components (or combinations thereof) selected from the group consisting of one or more recombination proteins (e.g., Int) or auxiliary factors (e.g., IHF and/or Xis) or combinations thereof, one or more compositions comprising one or more recombination proteins or auxiliary factors or combinations thereof (for example, Gateway® LR CLONASE™ Enzyme Mix (Invitrogen Corp., Carlsbad, Calif. Cat. No. 11791-019), Gateway® LR Clonase™ Plus enzyme mix (Invitrogen Corp., Carlsbad, Calif. Cat. No. 12538-013) or Gateway® BP CLONASE™ Enzyme Mix (Invitrogen Corp., Carlsbad, Calif. Cat. No. 11789-013)) one or more Destination Vector molecules (including those described herein), one or more Entry Clone or Entry Vector molecules (including those described herein), one or more primer nucleic acid molecules (particularly those described herein), one or more host cells (e.g., competent cells, such as E. coli cells, yeast cells, animal cells (including mammalian cells, insect cells, nematode cells, avian cells, fish cells, etc.), plant cells, and most particularly E. coli DB3, DB3.1 (e.g., E. coli LIBRARY EFFICIENCY® DB3.1™ Competent Cells; Invitrogen Corp., Carlsbad, Calif.), DB4 and DB5; see U.S. application Ser. No. 09/518,188, filed on Mar. 2, 2000, the disclosure of which is incorporated by reference herein in its entirety), and the like.

In related aspects, kits of the invention may comprise one or more nucleic acid molecules encoding at least one modified intein or a functional derivative or homolog thereof of the invention in conjunction with one or more recombination sites or portions thereof, one or more topoisomerase recognition sites or portions thereof and/or one or more topoisomerases such as one or more nucleic acid molecules comprising a nucleotide sequence encoding the one or more recombination sites (or portions thereof), one or more topoisomerase recognition sites (or portions thereof) of the invention, and particularly one or more of the nucleic acid molecules contained in the deposited clones described herein. Kits according to this aspect of the invention may also comprise one or more isolated nucleic acid molecules used in the invention, one or more vectors of the invention, one or more primer nucleic acid molecules used in the invention, and/or one or more antibodies of the invention.

Kits of the invention may further comprise one or more additional containers containing one or more additional components useful in combination with the nucleic acid molecules, polypeptides, vectors, host cells, or antibodies of the invention, such as one or more buffers, one or more detergents, one or more polypeptides having nucleic acid polymerase activity, one or more polypeptides having reverse transcriptase activity, one or more transfection reagents, one or more nucleotides, and the like. In a related aspect the kits of the invention may comprise one or more reagents for selection such as enzymes, substrates, ligands, inhibitors, labels, antibodies, probes or primers. Such kits may be used in any process advantageously using the nucleic acid molecules, primers, vectors, host cells, polypeptides, antibodies and other compositions used in or selected by the invention, for example in methods of synthesizing nucleic acid molecules (e.g., via amplification such as via PCR), in methods of cloning nucleic acid molecules (e.g., via recombinational cloning as described herein), and the like. The kits of the invention may also comprise instructions for carrying out the various methods of the invention.

### EXAMPLES

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Example 1

### Introduction

Recently, two novel cloning techniques have been developed by Invitrogen Corporation which eliminate the need for restriction enzymes and ligase in the construction of expression plasmids. These systems, the Gateway® and Topo® systems (Invitrogen, Carlsbad, Calif.), use site-specific recombination and topoisomerase-based cloning to precisely insert target genes into one of a number of available expression vectors. These systems effectively eliminate the need for conventional restriction-ligation cloning, allowing genes to be cloned and optimized for expression much more rapidly. An important aspect of these systems is that they can be used to automate the cloning and expression of many genes in a highly parallel format. This capability is essential for high-throughput applications.

During the same time, a novel purification method based on self-cleaving affinity tags was also being developed. This method exploits a recently discovered self-splicing protein element, known as an intein, to greatly simplify the purification of affinity-tagged product proteins. In practice, a target protein is fused to an intein-linked affinity tag and purified by a conventional affinity separation. Once purified however, the intein triggers the release of the product protein from the affinity tag in a self-cleaving reaction. The result is a highly purified product from a single column step, and because the cleavage takes place on column and without exogenous protease, no further purification of the product is required. As with the Gateway® and Topo® systems, self-cleaving affinity tags can allow automated purification of large numbers of proteins in parallel due to the generality of the purification protocol.

The research described here combines the Gateway® and Topo® cloning systems with intein-mediated protein purification technology. The goal is to provide both rapid cloning capability as well as a rapid, simple purification method for the expressed product. Ultimately a system comprising a Topo® based entry vector and a series of Gateway® vectors with various combinations of promoters, affinity tags and inteins will be designed to allow researchers to rapidly optimize the cloning and purification of various genes and their products. It is anticipated that this combination of technology will find applications in high-throughput cloning and characterization of newly discovered DNA sequences, as well as gene library cloning and characterization. For smaller applications, this combination will accelerate the cloning and characterization of individual proteins, and the flexibility of the Gateway system will allow the rapid optimization of protein expression with self-cleaving affinity tags.

Generation of the Topo and Gateway Inteins

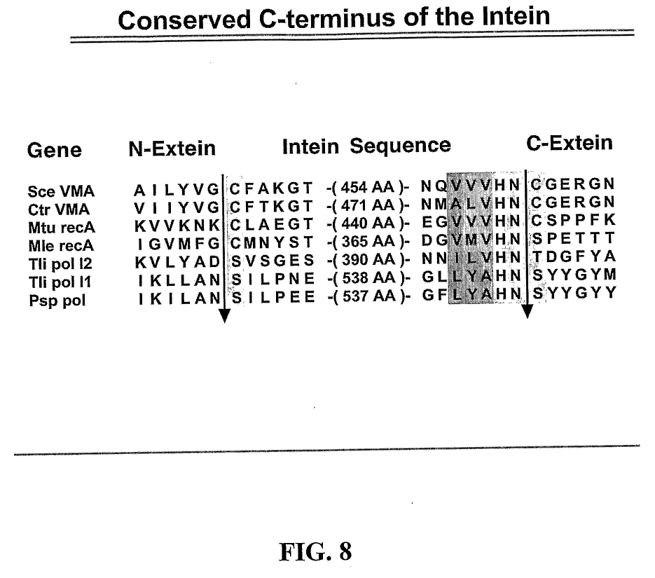

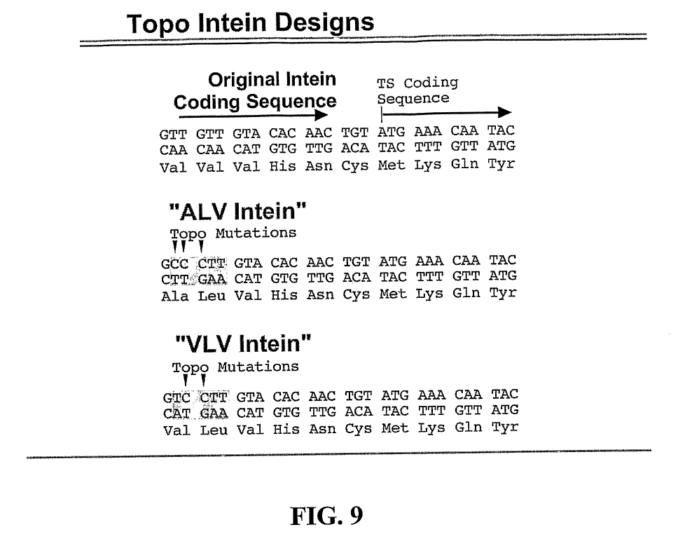

A key requirement for intein-mediated protein purification is that the initial amino acid of the product protein must immediately follow the highly conserved histidine-asparagine dipeptide at the C-terminus of the intein (FIG. 8). In the prior published mini-intein system (PCT application No. PCT/US00/22581 (WO 01/12820)), this requirement is fulfilled by a translationally silent restriction site close to the C-terminus of the intein, which allows the target protein's DNA sequence to be PCR amplified and inserted immediately adjacent to the intein sequence without modification of the target protein or intein. In the Topo recognition sequence and Gateway recombination site-based modified inteins, this requirement is met, inter alia, by the modification of the intein to include the required Topo recognition sequence and/or Gateway recombination site within (e.g., embedded) the coding sequence of the intein. In other embodiments, the modification of the intein comprises incorporation of the required Topo recognition sequence and/or Gateway recombination site adjacent to (e.g., 1, 2, 3, 4, 5, 6, 7, 8, 9, 10, 11, 12, 13, 14, 15, 16, 17, 18, 19, 20, and up to and including for example 100 or more nucleotides from the 5′ or 3′ end of the nucleotide sequence of the intein). To minimize the length of the PCR primers used to initially amplify the product gene, and therefore the ultimate cost of each strategy, the Topo sequence has been inserted very close to the C-terminus of the intein. This goal has been achieved through the modification of the ΔI-CM intein to include the Topo target sequence at its C-terminus (FIG. 9), and the insertion of the Gateway attB1 into the part of the intein where the endonuclease domain sequence was removed.

In principle, any intein capable of functioning with a deleted endonuclease domain would still be able to retain activity with any of the gateway attB1 sequences inserted in place of the endonuclease domain. In addition, any intein capable of cleaving and functioning with the native end modified to enable topo-cloning (e.g., Directional Topo, Topo Cloning, and Topo Tools) with any of the topoisomerase recognition sequences may be employed with the compositions and methods of this invention. This is due to the intein splicing and/or cleaving mechanisms being similar for all inteins. The modified Mtu RecA inteins described above demonstrates that any gateway aft recombination sequence (e.g., one or more of the representative att recombination sequences depicted in Table 2, supra) can be inserted into the deleted endonuclease domain for any intein (for example, into one or more of the representative inteins depicted in Appendix A, as well as in any intein in the intein registry or functional derivative or homolog thereof). Thus, the Topo recognition sequence and/or recombination site modifications described herein applies over the full range of inteins.

Materials and Methods

Engineering the Intein Topo and Gateway Inteins

The engineered Topo, Gateway and Topo+Gateway inteins were generated by modification of the original Mtu RecA mini cleaving (ΔI-CM) intein. Oligonucleotide primers were designed to make the necessary insertions and mutations. Of the many attB sequences to choose from, the inventors arbitrarily choose to utilize ACA AGT TTG TAC AAA AAA GCA GGC AGC (e.g., attB1 recombination sequence) as a starting point to rationally engineer the Gateway intein. The backwards primer JH001 was synthesized to contain the attB1 sequence above as well as a unique BssH II site for inserting the attB1 sequence into the ΔI-CM intein region that formerly contained the endonuclease domain. Backwards primer JH003 was designed to modify the native end of the intein to contain the topoisomerase recognition sequence TCCTT to enable topo-cloning. The insertion of this Topo recognition sequence changes the native intein C-terminal amino acid sequence from VVVHN to VLVHN. Other Topo recognition sequences employed with the Invitrogen Corporation's Directional Topo, Topo Cloning, and Topo Tools-based topoisomerase cloning reactions could be used in combination with other mutations or other inteins to make additional Topo-capable inteins.

TABLE 6
Primers For Intein Engineering

| | |
|---|---|
| Primer | |
| Name | Primer Sequence (5′→3′) |

| | | |
|---|---|---|
| Gateway attB1 | JH001 | CTG CAC GCG CGC GCT GCC TGC TTT |
| Primer | | TTT GTA CAA ACT TGT CGG AAT CGG |
| | | CGC ACT GTC ACC GAA TCC |
| | | (SEQ ID NO. X) |
| Topo Reverse | JH004 | GGT ATT GTT TAC AGT TGT GTA CAA |
| Primer | | GGA CCC CTT CGG C |
| | | (SEQ ID NO. X) |

The insertions and modifications were made through PCR amplification of the ΔI-CM intein with the described oligonucleotide primers. Conventional cloning methods using restriction enzyme digestion and ligation were used to insert the Gateway recombination site and Topo recognition sequence inteins into the pMIT genetic selection context (Wood, D. W., Wu, W., Belfort, G., Derbyshire, V. & Belfort, M. (1999) A genetic system yields self-cleaving inteins for bioseparations. Nat Biotechnol 17, 889-92.). This context results in the expression of the target intein in fusion to the thymidylate synthase enzyme for phenotypic evaluation of intein cleaving in E. coli strain D1210ΔthyA.

The cleaving activities of the engineered inteins were initially evaluated by phenotype analysis in the context of the thymidylate synthase selection system (Wood, D. W. et al. Nat Biotechnol 17, 889-92 (1999)). Plasmids containing the engineered inteins in fusion to the maltose binding protein and thymidylate synthase were transformed into D1210ΔthyA cells. Selection was done on minimal plates supplemented with ampicillin. Only inteins that were capable of cleaving the thymidylate synthase product enzyme efficiently can survive under these conditions, and intein activity is therefore reflected in the growth phenotype of the expressing cells. Once cleaving capability was confirmed, all sequences were confirmed by sequencing.

Expression and Purification of Intein Precursor Proteins

The recombinant plasmids containing the engineered inteins were transformed into E. coli strain ER2566 {F-l-fhuA2 [lon] ompT lacZ::T7 gene1 gal sulA11Δ(mcrC-mrr)114::IS10 R(mcr-73::miniTn10-Tet^{S})2 R(zgb-210::Tn10-Tet^{S}) endA1[dcm]} (New England Biolabs, Beverly, Mass.). The culture was grown in LB broth plus ampicillin at 37° C. When the cell culture reached mid-log phase (OD₆₀₀=0.5), the culture was then transferred to a 15-20° C. shaker for 30 min to allow the culture to equilibrate. Isopropylthiogalactoside (IPTG, Sigma, St. Louis, Mo.) was then added to induce precursor production.

After a 4-8 hour induction, the cell culture was harvested by centrifugation. The culture was spun down at 4° C. at 4000×g and the media removed. Cells were then resuspended in cold column buffer and allowed to undergo a freeze-thaw cycle, usually overnight, before sonication. Cells were lysed by short 15-second sonication pulses for a total of 1 minute. The lysed sample was then centrifuged at 9000×g for 30 minutes at 4° C. The lysate was diluted 1:5 with column buffer before being loaded onto amylose resin that had been equilibrated with column buffer. Once the precursor was loaded onto the column, column buffer was used to wash other proteins and undesired matter through the column.

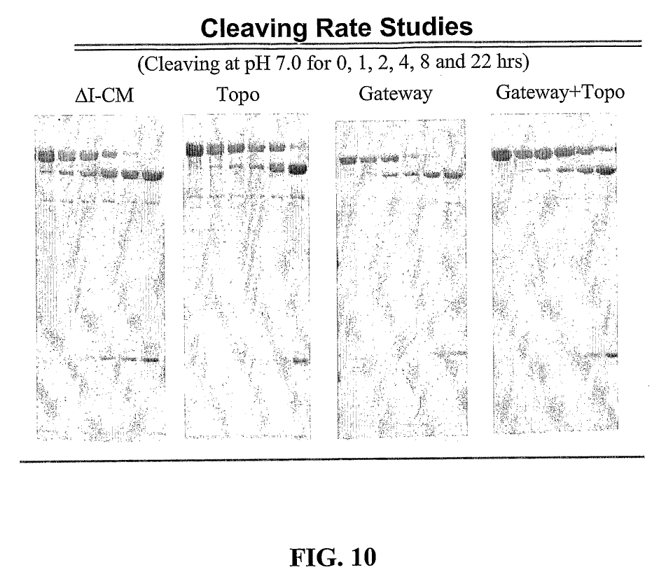

Purification at this point can take one of two methods. To purify precursor for in vitro studies, column buffer with the addition of 10 mM maltose would is used to elute the precursor protein from the column. The eluted fractions contain the uncleaved precursor comprised of the binding domain-intein-target protein fusion. This method was used to purify the precursor protein used for cleaving studies in all cases (FIGS. 10 and 11, respectively).

To purify only the target protein, column buffer with the pH shifted to a lower value than the original pH of 8.5 would be eluted through. The eluted fractions would thus contain target protein only. The cleaved binding domain and intein could then be removed from the affinity column with column buffer containing maltose. The affinity column was then regenerated using a wash protocol as defined by the manufacturer.

Various column buffers can be used for the purification process. Many buffers with varying ionic strengths, oxidative potential, pH have been utilized. Various combinations of the following ranges have been used as the column buffer and has no effect on intein activity: PIPES (0-200 mM), Bis-Tris (0-200 mM), Tris (0-200 mM), EDTA (0-10 mM), DTT (0-10 mM), NaCl (0-500 mM), AMPD (0-20 mM), Lyzozyme, DNase, RNase, glycerol (0-5%), with the pH adjusted to ranges from 6.0 to 8.5. For example, the column buffer used for the proof of principle experiment with aFGF as the test protein was composed of 20 mM AMPD (2-amino-2-methyl-1,3-propanediol), 20 mM PIPES, 200 mM NaCl, 1 mM DTT, 2 mM EDTA, 5% glycerol and pH adjusted to 8.5. The temperatures where cleaving purification, cleaving and splicing experiments were conducted ranged from 4° C. to 37° C.

In Vitro Experiments

Purified precursor was obtained as described above. Reactions for cleaving were set up at 4° C. to minimize cleaving. Acid was added to shift the pH of the cleaving reaction buffer to lower values to induce intein cleaving. Samples were then held at various temperatures to determine cleavage rates of the inteins at relative to pH and temperature. At various time intervals, 15 μl samples were removed and prepared for protein gel analysis.

In Vivo Experiments

Fresh LB+Amp media was inoculated with 1:100 saturated overnight culture of transformed ER2566 containing the engineered intein plasmids. Cultures were allowed to grow until OD₆₀₀=0.5 was reached at 37° C. The cell cultures were then moved to a 20° C. bath to equilibrate the cultures before being induced with IPTG. After induction, the cultures were moved into various shaker baths set a different temperatures so that in vivo cleavage of the intein could be determined. The optical density at time of induction was noted so that samples could be normalized. 1 ml samples were collected at various time intervals and samples were diluted so that the optical density was the same. Collected samples were centrifuged and the media removed. Samples were then prepared for protein gel analysis by resuspending in lysis buffer and sonicating. The lysed sample was then centrifuged to separate cell debris from the desired soluble protein fraction. 15 μl of the lysate was collected with classic loading buffer and prepped for loading on the protein gels.

Results and Discussion

The engineered Topo recognition sequence and Gateway recombination site inteins, as well as the double Gateway recombination site+Topo recognition sequence intein, have been evaluated in a number of simple tests for basic cleaving activity and controllability. The first test is based on the resulting phenotype when the modified engineered inteins are inserted into the thymidylate synthase selection system context. As discussed before, an acceptable intein for cleaving is one that, for example, yields a positive growth phenotype on a defined thymineless medium in this system. Each of the three engineered inteins, along with three control inteins, were cloned into this system and their phenotypes where evaluated (see page 24 of Appendix B, infra). It was observed that the engineered modified inteins exhibited phenotypes similar to the original ΔI-CM intein, although the growth of both inteins with the Topo recognition sequence mutations seemed to be slightly less positive. This demonstrates that the engineered inteins are practical for the generation of self-cleaving affinity tags for use in the compositions and methods of the invention.

The second test of the engineered modified inteins is expression of a precursor protein with each intein in vivo at a range of temperatures. If the modified inteins are controllable and useful for purification, the modified inteins should exhibit very little cleaving at low temperature, but nearly complete cleaving of the precursor at higher temperature. Evidence that little or no uncleaved precursor is generated is provided by the purified precursor lanes shown in FIGS. 10 and 11, respectively. In all cases, the zero-time samples (the first lane of each panel in FIG. 10 and lanes 2, 8 and 14 in FIG. 11) show only the presence of precursor. Any prematurely cleaved binding tag would co-purify with the precursor and show up in these lanes at the same molecular weight as the cleaved tag in the later time points. The lack of cleaved binding tags indicates that it is absent at the beginning of the purification, and therefore premature cleavage has not occurred. In this example, the protein of interest is the aFGF protein.

The final test of the engineered inteins is to see whether they are pH and temperature controllable in vitro and what range of cleaving rates can be attained for each of them. For these results, precursor protein was expressed for fusions of each intein to maltose binding protein (e.g., one example of a sequence or affinity tag used in the methods of the present invention) and a standard acidic fibroblast growth factor test protein (e.g., one example of a protein of interest that may be purified using the compositions and methods of the present invention). The uncleaved precursor proteins were subjected to various combinations of pH and temperature, and samples were taken over time to follow the cleaving reaction of each intein (FIGS. 10 and 11, respectively). It was observed that the Topo recognition sequence mutations resulted in only a slight loss of cleaving activity for both inteins where they appear, however the Gateway recombination site insertion at the center of the intein (e.g., an embedded recombination site) does not appear to affect cleaving at all. The data summarizing the intein cleaving rates at various temperatures and pH are provided in Table 7 below. The result is that the modified inteins are capable of rapid and controllable cleaving to completion. Thus, modified inteins have utility as linkers for the generation of practical self-cleaving affinity tags for high throughput applications and/or ultrahigh throughput applications.

TABLE 7
Intein Cleaving Rates at Various Temperatures and pH.

| | pH 8.5 | pH 7.5 | pH 7.0 | pH 6.5 | pH 6.0 |
|---|---|---|---|---|---|
| 37° C. | ΔI-CM = 30 | ΔI-CM = 7 | ΔI-CM = 3 | ΔI-CM = 1.5 | ΔI-CM = 1.5 |
| | Topo = 150 | Topo = ? | Topo = 7 | Topo = ? | Topo = 4.6 |
| | Gate = 50 | Gate = ? | Gate = 3 | Gate = ? | Gate = 1.5 |
| | T + G = 150 | T + G = 30 | T + G = 8 | T + G = 4 | T + G = 3 |
| 23° C. | ΔI-CM = >150 | | ΔI-CM = 10 | | ΔI-CM = 6.5 |
| | Topo = >300 | | Topo = 40 | | Topo = 16 |
| | Gate = >300 | | Gate = 16 | | Gate = 6 |
| | T + G = ? | | T + G = ? | | T + G = ? |
| 4° C. | ΔI-CM = >300 | | ΔI-CM = 150 | | ΔI-CM = 150 |
| | Topo = >300 | | Topo = >300 | | Topo = >300 |
| | Gate = >300 | | Gate = 150 | | Gate = 150 |
| | T + G = ? | | T + G = ? | | T + G = ? |

For each intein, the approximate cleaving half life is indicated in hours. The product protein in this case is the aFGF test protein described in Wood et al., Biotechnology Progress vol. 16, pp. 1055-1063.). The ΔI-CM data is provided for by way of comparison.

### REFERENCES

1. Hirata, R., Ohsumk, Y., Nakano, A., Kawasaki, H., Suzuki, K. & Anraku, Y. Molecular Structure Of A Gene, Vma1, Encoding The Catalytic Subunit Of H(+)-Translocating Adenosine Triphosphatase From Vacuolar Membranes Of Saccharomyces Cerevisiae. J Biol Chem 265, 6726-33 (1990).
2. Perler, F. B. Inbase: The Intein Database. Nucleic Acids Res 30, 383-4 (2002).
3. Perler, F. B., Davis, E. O., Dean, G. E., Gimble, F. S., Jack, W. E., Neff, N., Noren, C. J., Thorner, J. & Belfort, M. Protein Splicing Elements: Inteins And Exteins—A Definition Of Terms And Recommended Nomenclature. Nucleic Acids Res 22, 1125-7 (1994).
4. Belfort, M., Reaban, M. E., Coetzee, T. & Dalgaard, J. Z. Prokaryotic Introns And Inteins: A Panoply Of Form And Function. J Bacteriol 177, 3897-903 (1995).
5. Gimble, F. S. & Thorner, J. Homing Of A Dna Endonuclease Gene By Meiotic Gene Conversion In Saccharomyces Cerevisiae. Nature 357, 301-6 (1992).
6. Doolittle, R. F. & Bork, P. Evolutionarily Mobile Modules In Proteins. Sci Am 269, 50-6 (1993).
7. Belfort, M. & Perlman, P. S. Mechanisms Of Intron Mobility. J Biol Chem 270, 30237-40 (1995).
8. Davis, E. O., Jenner, P. J., Brooks, P. C., Colston, M. J. & Sedgwick, S. G. Protein Splicing In The Maturation Of M. Tuberculosis Reca Protein: A Mechanism For Tolerating A Novel Class Of Intervening Sequence. Cell 71, 201-10 (1992).
9. Perler, F. B., Comb, D. G., Jack, W. E., Moran, L. S., Qiang, B., Kucera, R. B., Benner, J., Slatko, B. E., Nwankwo, D. O., Hempstead, S. K. & Et Al. Intervening Sequences In An Archaea Dna Polymerase Gene. Proc Natl Acad Sci USA 89, 5577-81 (1992).
10. Gu, H. H., Xu, J., Gallagher, M. & Dean, G. E. Peptide Splicing In The Vacuolar Atpase Subunit A From Candida Tropicalis. J Biol Chem 268, 7372-81 (1993).
11. Liu, X. Q. & Hu, Z. Identification And Characterization Of A Cyanobacterial Dnax Intein. Febs Lett 408, 311-4 (1997).
12. Cooper, A. A., Chen, Y. J., Lindorfer, M. A. & Stevens, T. H. Protein Splicing Of The Yeast Tfp1 Intervening Protein Sequence: A Model For Self-Excision. Embo J 12, 2575-83 (1993).
13. Derbyshire, V., Wood, D. W., Wu, W., Dansereau, J. T., Dalgaard, J. Z. & Belfort, M. Genetic Definition Of A Protein-Splicing Domain: Functional Mini-Inteins Support Structure Predictions And A Model For Intein Evolution. Proc Natl Acad Sci USA 94, 11466-71 (1997).
14. Wu, W., Wood, D. W., Belfort, G., Derbyshire, V. & Belfort, M. Intein-Mediated Purification Of Cytotoxic Endonuclease I-Tevi By Insertional Inactivation And Ph-Controllable Splicing. Nucleic Acids Res 30, 4864-71 (2002).
15. Duan, X., Gimble, F. S. & Quiocho, F. A. Crystal Structure Of Pi-Scei, A Homing Endonuclease With Protein Splicing Activity. Cell 89, 555-64 (1997).
16. Ichiyanagi, K., Ishino, Y., Ariyoshi, M., Komori, K. & Morikawa, K. Crystal Structure Of An Archaeal Intein-Encoded Homing Endonuclease Pi-Pfui. J Mol Biol 300, 889-901 (2000).
17. Chong, S. & Xu, M. Q. Protein Splicing Of The Saccharomyces Cerevisiae Vma Intein Without The Endonuclease Motifs. J Biol Chem 272, 15587-90 (1997).
18. Chong, S., Shao, Y., Paulus, H., Benner, J., Perler, F. B. & Xu, M. Q. Protein Splicing Involving The Saccharomyces Cerevisiae Vma Intein. The Steps In The Splicing Pathway, Side Reactions Leading To Protein Cleavage, And Establishment Of An In Vitro Splicing System. J Biol Chem 271, 22159-68 (1996).
19. Shao, Y. & Kent, S. B. Protein Splicing: Occurrence, Mechanisms And Related Phenomena. Chem. Biol 4, 187-94 (1997).
20. Paulus, H. Protein Splicing And Related Forms Of Protein Autoprocessing. Annu Rev Biochem 69, 447-96 (2000).
21. Perler, F. B., Xu, M. Q. & Paulus, H. Protein Splicing And Autoproteolysis Mechanisms. Curr Opin Chem Biol 1, 292-9 (1997).
22. Shingledecker, K., Jiang, S. & Paulus, H. Reactivity Of The Cysteine Residues In The Protein Splicing Active Center Of The Mycobacterium Tuberculosis Reca Intein. Arch Biochem Biophys 375, 138-44 (2000).
23. Chong, S., Williams, K. S., Wotkowicz, C. & Xu, M. Q. Modulation Of Protein Splicing Of The Saccharomyces Cerevisiae Vacuolar Membrane Atpase Intein. J Biol Chem 273, 10567-77 (1998).
24. Xu, M. Q. & Perler, F. B. The Mechanism Of Protein Splicing And Its Modulation By Mutation. Embo J 15, 5146-53 (1996).
25. Wood, D. W., Wu, W., Belfort, G., Derbyshire, V. & Belfort, M. A Genetic System Yields Self-Cleaving Inteins For Bioseparations. Nat Biotechnol 17, 889-92 (1999).

### EQUIVALENTS

The invention illustratively described herein suitably may be practiced in the absence of any element or elements, limitation or limitations which is not specifically disclosed herein. Thus, for example, in each instance herein any of the terms “comprising,” “consisting essentially of,” and “consisting of” may be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed herein, optional features, modification and variation of the concepts herein disclosed may be resorted to by those skilled in the art, and that such modifications and variations are considered to be within the scope of this invention as defined by the appended claims. In addition, where features or aspects of the invention are described in terms of Markush groups, those skilled in the art will recognize that the invention is also thereby described in terms of any individual member or subgroup of members of the Markush group.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other aspects of the invention are within the following claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference. The entire disclosures of U.S. application Ser. No. 10/640,422 filed Aug. 14, 2003; U.S. application Ser. No. 09/517,466 filed Mar. 2, 2000; U.S. application Ser. No. 09/732,914, filed Dec. 11, 2000; U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/233,492, filed Jan. 20, 1999; U.S. Pat. No. 6,143,557; U.S. appl. Ser. No. 60/065,930, filed Oct. 24, 1997; U.S. application Ser. No. 09/177,387 filed Oct. 23, 1998; U.S. application Ser. No. 09/296,280, filed Apr. 22, 1999; U.S. application Ser. No. 09/296,281, filed Apr. 22, 1999; U.S. appl. Ser. No. 60/108,324, filed Nov. 13, 1998; U.S. application Ser. No. 09/438,358, filed Nov. 12, 1999; U.S. application Ser. No. 09/695,065, filed Oct. 25, 2000; U.S. application Ser. No. 09/432,085 filed Nov. 2, 1999; U.S. appl. Ser. No. 60/122,389, filed Mar. 2, 1999; U.S. appl. Ser. No. 60/126,049, filed Mar. 23, 1999; U.S. appl. Ser. No. 60/136,744, filed May 28, 1999; U.S. appl. Ser. No. 60/122,392, filed Mar. 2, 1999; and U.S. appl. Ser. No. 60/161,403, filed Oct. 25, 1999, are herein incorporated by reference.

APPENDIX A

| | | | | | |
|---|---|---|---|---|---|
| | | | Prototype | | |
| Intein Name | Class | Extein | Allele | Organism Name | Organism Description |

Eucarya

| | | | | | |
|---|---|---|---|---|---|
| Cba PRP8 | Theo. | PRP8, pre-mRNA | Fne PRP8 | Cryptococcus | Yeast, human pathogen |
| | | splicing factor | (Cne | bacillisporus (aka | |
| | | | PRP8) | Cryptococcus neoformans | |
| | | | | gattii) | |
| Ceu ClpP | Exp. | ClpP protease | | Chlamydomonas | Green alga, taxon: 3053 |
| | | | | eugametos (chloroplast) | |
| CIV RIR1 | Theo. | Ribonucleoside- | Pfu RIR1-2 | Chilo iridescent virus | dsDNA eucaryotic virus, |
| | | diphosphate | | | taxon: 10488 |
| | | reductase, alpha | | | |
| | | subunit | | | |
| Ctr VMA | Exp. | Vacuolar ATPase | Sce VMA | Candida tropicalis | Yeast |
| | | (H+-transporting | | (nucleus) | |
| | | ATP synthase), | | | |
| | | subunit A | | | |
| Fne-A PRP8 | Theo. | PRP8, pre-mRNA | Fne PRP8 | Filobasidiella neoformans | Yeast, human pathogen |
| (Cne-A | | splicing factor | (Cne | (Cryptococcus | |
| PRP8) | | | PRP8) | neoformans) Serotype A, | |
| | | | | PHLS_8104 | |
| Fne-AD PRP8 | Theo. | PRP8, pre-mRNA | Fne PRP8 | Filobasidiella neoformans | Yeast, human pathogen, |
| (Cne-AD | | splicing factor | (Cne | (Cryptococcus | ATCC32045, taxon: 5207 |
| PRP8) | | | PRP8) | neoformans), Serotype | |
| | | | | AD, CBS132). | |
| Gth DnaB | Theo. | DnaB helicase | Ssp DnaB | Guillardia theta (plastid) | Cryptophyte Algae |
| Kla VMA | Theo. | Vacuolar ATPase | Sce VMA | Kluyveromyces lactis, | Yeast, taxon: 28985 |
| | | (H+-transporting | | strain CBS 683 | |
| | | ATP synthase), | | | |
| | | subunit A | | | |
| Kpo VMA | Theo. | Vacuolar ATPase | Sce VMA | Kluyveromyces | Yeast, taxon: 36033 |
| | | (H+-transporting | | polysporus, strain CBS | |
| | | ATP synthase), | | 2163 | |
| | | subunit A | | | |
| Ppu DnaB | Theo. | DnaB helicase | Ssp DnaB | Porphyra purpurea | Red Alga |
| | | | | (chloroplast) | |
| Sca VMA | Theo. | Vacuolar ATPase | Sce VMA | Saccharomyces castellii, | Yeast, taxon: 27288 |
| | | (H+-transporting | | strain CBS 4309 | |
| | | ATP synthase), | | | |
| | | subunit A | | | |
| Sce VMA | Exp. | Vacuolar ATPase | Sce VMA | Saccharomyces cerevisiae | Yeast |
| | | (H+-transporting | | (nucleus) | |
| | | ATP synthase), | | | |
| | | subunit A | | | |
| Sda VMA | Theo. | Vacuolar ATPase | Sce VMA | Saccharomyces | Yeast, taxon: 27289 |
| | | (H+-transporting | | dairenensis, strain CBS | |
| | | ATP synthase), | | 421 | |
| | | subunit A | | | |
| Sex VMA | Theo. | Vacuolar ATPase | Sce VMA | Saccharomyces exiguus, | Yeast, taxon: 34358 |
| | | (H+-transporting | | strain CBS 379 | |
| | | ATP synthase), | | | |
| | | subunit A | | | |
| Sun VMA | Theo. | Vacuolar ATPase | Sce VMA | Saccharomyces unisporus, | Yeast, taxon: 27294 |
| | | (H+-transporting | | strain CBS 398 | |
| | | ATP synthase), | | | |
| | | subunit A | | | |
| Tgl VMA | Theo. | Vacuolar ATPase | Sce VMA | Torulaspora globosa, | Yeast, taxon: 48254 |
| | | (H+-transporting | | strain CBS 764 | |
| | | ATP synthase), | | | |
| | | subunit A | | | |
| Tpr VMA | Theo. | Vacuolar ATPase | Sce VMA | Torulaspora pretoriensis, | Yeast, taxon: 35629 |
| | | (H+-transporting | | strain CBS 5080 | |
| | | ATP synthase), | | | |
| | | subunit A | | | |
| Zba VMA | Theo. | Vacuolar ATPase | Sce VMA | Zygosaccharomyces bailii, | Yeast, taxon: 4954 |
| | | (H+-transporting | | strain CBS 685 | |
| | | ATP synthase), | | | |
| | | subunit A | | | |
| Zbi VMA | Theo. | Vacuolar ATPase | Sce VMA | Zygosaccharomyces | Yeast, taxon: 4957 |
| | | (H+-transporting | | bisporus, strain CBS 702 | |
| | | ATP synthase), | | | |
| | | subunit A | | | |
| Zro VMA | Theo. | Vacuolar ATPase | Sce VMA | Zygosaccharomyces | Yeast, taxon: 4956 |
| | | (H+-transporting | | rouxii, strain CBS 688 | |
| | | ATP synthase), | | | |
| | | subunit A | | | |

Eubacteria

| | | | | | |
|---|---|---|---|---|---|
| Aae RIR2 | Theo. | Ribonucleoside- | | Aquifex aeolicus strain | Thermophilic |
| | | diphosphate | | VF5 | chemolithoautotroph, |
| | | reductase, beta | | | taxon: 63363 |
| | | subunit | | | |
| APSE1 dpol | Theo. | DNA polymerase I | | Bacteriophage of | Bacteriophage, taxon: 106199 |
| | | (Pol I family) | | endosymbiot of | |
| | | | | Acyrthosiphon pisum | |
| Bsu-M1918 | Theo. | Ribonucleoside- | Pfu RIR1-2 | B. subtilis M1918 | Prophage in B. subtilis M1918. |
| RIR1 | | diphosphate | | (prophage) | taxon: 157928 |
| | | reductase, alpha | | | |
| | | subunit | | | |
| Bsu-SPBc2 | Theo. | Ribonucleoside- | Pfu RIR1-2 | B. subtilis strain 168 Sp | B. subtilis taxon 1423. SPbeta |
| RIR1 | | diphosphate | | beta c2 prophage | c2 phage, taxon: 66797 |
| | | reductase, alpha | | | |
| | | subunit | | | |
| Cau Hyp | Theo. | Hypothetical protein | | Chloroflexus aurantiacus | Anoxygenic phototroph, Strain |
| | | | | | J-10-fl, taxon: 1108 |
| Cbu DnaB | Theo. | DnaB helicase | Ssp DnaB | Coxiella burnetii RSA 493 | Proteobacteria; Legionellales; |
| | | | | | taxon: 227377 |
| Cth Hyp | Theo. | Hypothetical protein | | Clostridium thermocellum | ATCC27405, taxon: 203119 |
| Dha RIR1 | Theo. | Ribonucleoside- | Pfu RIR1-2 | Desulfitobacterium | Anaerobic dehalogenating |
| | | diphosphate | | hafniense DCB-2 | bacteria, taxon: 49338 |
| | | reductase, alpha | | | |
| | | subunit | | | |
| Dra RIR1 | Theo. | Ribonucleoside- | Pfu RIR1-2 | Deinococcus radiodurans | Radiation resistant, taxon: 1299 |
| | | diphosphate | | R1, TIGR strain | |
| | | reductase, alpha | | | |
| | | subunit | | | |
| Dra Snf2 | Theo. | SNF2/Rad54 | Dra Snf2 | Deinococcus radiodurans | Radiation and DNA damage |
| | | helicase | | R1, TIGR strain | resistent, taxon: 1299 |
| Dra- | Exp. | SNF2/Rad54 | Dra Snf2 | Deinococcus radiodurans | Radiation and DNA damage |
| ATCC13939 | | helicase | | R1, ATCC13939/Brooks | resistent, taxon: 1299 |
| Snf2 | | | | &Murray strain | |
| Mav DnaB | Exp. | DnaB helicase | Mle DnaB | Mycobacterium avium | ATCC35712, taxon 1764 |
| Mbo DnaB | Theo. | DnaB helicase | Ssp DnaB | Mycobacterium bovis | strain = “AF2122/97”, |
| | | | | subsp. bovis | taxon: 233413 |
| Mbo Pps1 | Theo. | Pps1, YC24 family | Mtu Pps1 | Mycobacterium bovis | strain = “AF2122/97”, |
| | | of ABC transporters | | subsp. bovis | taxon: 233413 |
| Mch RecA | Theo. | RecA | Mle RecA | Mycobacterium chitae | IP14116003, taxon: 1792 |
| Mcjw1 | Theo. | DnaB helicase | | Mycobacteriophage CJW1 | Bacteriophage, taxon: 205869 |
| DnaB | | | | | |
| Mfa RecA | Theo. | RecA | Mle RecA | Mycobacterium fallax | CITP8139, taxon: 1793 |
| Mfl GyrA | Exp. | DNA gyrase subunit A | Mle GyrA | Mycobacterium flavescens | taxon: 1776, reference #930991 |
| Mfl RecA | Exp. | RecA | Mle RecA | Mycobacterium flavescens | strain = Fla0, taxon: 1776, ref. |
| | | | | Fla0 | #930991 |
| Mfl- | Theo. | RecA | Mle RecA | Mycobacterium | strain = ATCC14474, taxon: 1776 |
| ATCC14474 | | | | flavescens, ATCC14474 | ref #930991 |
| RecA | | | | | |
| Mga GyrA | Theo. | DNA gyrase subunit A | Mle GyrA | Mycobacterium gastri | HP4389, taxon: 1777 |
| Mga Pps1 | Exp. | Pps1, YC24 family | | Mycobacterium gastri | HP4389, taxon: 1777 |
| | | of ABC transporters | | | |
| Mga RecA | Theo. | RecA | Mle RecA | Mycobacterium gastri | HP4389, taxon: 1777 |
| Mgo GyrA | Theo. | DNA gyrase subunit A | Mle GyrA | Mycobacterium gordonae | taxon: 1778, reference number |
| | | | | | 930835 |
| Min DnaB | Theo. | DnaB helicase | Mle DnaB | Mycobacterium | strain 1442, taxon: 1767 |
| | | | | intracellulare | |
| Mka GyrA | Theo. | DNA gyrase subunit A | Mle GyrA | Mycobacterium kansasii | taxon: 1768 |
| Mle DnaB | Exp. | DnaB helicase | Mle DnaB | Mycobacterium leprae, | Human pathogen, taxon: 1769 |
| | | | | strain TN | |
| Mle GyrA | Theo. | DNA gyrase subunit A | Mle GyrA | Mycobacterium leprae TN | Human pathogen, |
| | | | | | STRAIN = TN, taxon: 1769 |
| Mle Pps1 | Theo. | Pps1 (xheA), YC24 | | Mycobacterium leprae | Human pathogen, taxon: 1769 |
| | | family of ABC | | | |
| | | transporters | | | |
| Mle RecA | Exp. | RecA | Mle RecA | Mycobacterium leprae, | Human pathogen, taxon: 1769 |
| | | | | strain TN | |
| Mma GyrA | Theo. | DNA gyrase subunit A | Mle GyrA | Mycobacterium | taxon: 1780 |
| | | | | malmoense | |
| Momega | Theo. | DnaB helicase | | Mycobacteriophage | Bacteriophage, taxon: 205879 |
| DnaB | | | | Omega | |
| Msh RecA | Theo. | RecA | Mle RecA | Mycobacterium shimodei | ATCC27962, taxon: 29313 |
| Msm DnaB-1 | Theo. | DnaB helicase | Mle DnaB | Mycobacterium | MC2 155 |
| | | | | smegmatis MC2 155 | |
| Msm DnaB-2 | Theo. | DnaB helicase | Ssp DnaB | Mycobacterium | MC2 155 |
| | | | | smegmatis MC2 155 | |
| Mthe RecA | Theo. | RecA | Mle RecA | Mycobacterium | ATCC19527, taxon: 1797 |
| | | | | thermoresistibile | |
| Mtu Pps1 | Theo. | Pps1, YC24 family | Mtu Pps1 | Mycobacterium | Human pathogen, taxon: 83332 |
| | | of ABC transporters | | tuberculosis strains | |
| | | | | H37Rv &CDC1551 | |
| Mtu- | Exp. | DnaB helicase | Ssp DnaB | Mycobacterium | Human pathogen, taxon: 83332 |
| CDC1551 | | | | tuberculosis, CDC1551 | |
| DnaB | | | | | |
| Mtu-H37Rv | Exp. | DnaB helicase | Ssp DnaB | Mycobacterium | Human pathogen, taxon: 83332 |
| DnaB | | | | tuberculosis H37Rv | |
| Mtu-H37Rv | Exp. | RecA | | Mycobacterium | Human pathogen, taxon: 83332 |
| RecA | | | | tuberculosis H37Rv | |
| Mxe GyrA | Exp. | DNA gyrase subunit A | Mle GyrA | Mycobacterium xenopi | taxon: 1789 |
| Npu DnaB | Theo. | DnaB helicase | Ssp DnaB | Nostoc punctiforme | Cyanobacterium, taxon: 63737 |
| Npu DnaE | Theo. | DnaE pol subunit, | Ssp DnaE | Nostoc punctiforme | Cyanobacterium, taxon: 63737 |
| | | DNA polymerase III | | | |
| | | alpha subunit | | | |
| Npu GyrB | Theo. | DNA gyrase subunit B | | Nostoc punctiforme | Cyanobacterium, taxon: 63737 |
| Nsp DnaB | Theo. | DnaB helicase | Ssp DnaB | Nostoc species PCC7120, | Cyanobacterium, Nitrogen- |
| | | | | (Anabaena sp. PCC7120) | fixing, taxon: 103690 |
| Nsp DnaE | Theo. | DnaE pol subunit, | Ssp DnaE | Nostoc species PCC7120, | Cyanobacterium, Nitrogen- |
| | | DNA polymerase III | | (Anabaena sp. PCC7120) | fixing, taxon: 103690 |
| | | alpha subunit | | | |
| Nsp RIR | Exp. | Ribonucleoside | | Nostoc species PCC7120, | Cyanobacterium, Nitrogen- |
| | | Triphosphate | | (Anabaena sp. PCC7120) | fixing, taxon: 103690 |
| | | Reductase | | | |
| Rma DnaB | Exp. | DnaB helicase | Ssp DnaB | Rhodothermus marinus | Thermophile, taxon: 29549 |
| Spl DnaX | Theo. | DNA polymerase III | Ssp DnaX | Spirulina platensis, strain | Cyanobacteria, taxon: 1156 |
| | | gamma and tau | | C1 | |
| | | subunits | | | |
| Ssp DnaB | Exp. | DnaB helicase | Ssp DnaB | Synechocystis spp. strain | Cyanobacterium, taxon: 1148 |
| | | | | PCC6803 | |
| Ssp DnaE | Exp. | DnaE pol subunit, | Ssp DnaE | Synechocystis spp. strain | Cyanobacterium, taxon: 1148 |
| | | DNA polymerase III | | PCC6803 | |
| | | alpha subunit | | | |
| Ssp DnaX | Exp. | DNA pol III Tau- | Ssp DnaX | Synechocystis spp. strain | Cyanobacterium, taxon: 1148 |
| | | subunit | | PCC6803 | |
| Ssp GyrB | Theo. | DNA gyrase subunit B | Ssp GyrB | Synechocystis spp. strain | Cyanobacterium, taxon: 1148 |
| | | | | PCC6803 | |
| Tel DnaE | Theo. | DnaE pol subunit, | Ssp DnaE | Thermosynechococcus | Cyanobacterium, |
| | | DNA polymerase III | | elongatus BP-1 | |
| | | alpha subunit | | | |
| Ter DnaE | Theo. | DnaE pol subunit, | Ssp DnaE | Trichodesmium | Cyanobacterium, taxon: 203124 |
| | | DNA polymerase III | | erythraeum IMS101 | |
| | | alpha subunit | | | |
| Ter GyrB | Theo. | DNA gyrase subunit B | Ssp GyrB | Trichodesmium | Cyanobacterium, taxon: 203124 |
| | | | | erythraeum IMS101 | |
| Ter Snf2 | Theo. | SNF2/Rad54 | Dra Snf2 | Trichodesmium | Cyanobacterium, taxon: 203124 |
| | | helicase | | erythraeum IMS101 | |
| Tfus RecA-1 | Theo. | RecA | | Thermobifida fusca YX | Thermophile, taxon: 2021 |
| Tfus RecA-2 | Theo. | RecA | Mle RecA | Thermobifida fusca YX | Thermophile, taxon: 2021 |

Archaea

| | | | | | |
|---|---|---|---|---|---|
| Ape Hyp | Theo. | Hypothetical protein | | Aeropyrum pernix | Thermophile, taxon: 56636 |
| Fac Pps1 | Theo. | Pps1, YC24 family | Mtu Pps1 | Ferroplasma acidarmanus | strain fer1, eats |
| | | of ABC transporters | | | iron, taxon: 97393 |
| Fac RIR1 | Theo. | Ribonucleoside- | Pfu RIR1-2 | Ferroplasma acidarmanus | strain fer1, eats iron |
| | | diphosphate | | | |
| | | reductase, alpha | | | |
| | | subunit | | | |
| Hsp-NRC1 | Theo. | Cell division control | Pho | Halobacterium sp. NRC-1 | Halophile, taxon: 64091 |
| CDC21 | | protein 21 | CDC21-1 | | |
| Hsp-NRC1 | Theo. | DNA polymerase II, | Pho Pol II | Halobacterium sp. NRC-1 | Halophile, taxon: 64091 |
| Pol II | | DP2 subunit | | | |
| Mja GF-6P | Theo. | Glutamine-fructose- | | Methanococcus jannaschii | Thermophile, DSM 2661, |
| | | 6-phosphate | | | taxon: 2190 |
| | | transaminase | | | |
| Mja Helicase | Theo. | Putative SKI2-family | | Methanococcus jannaschii | Thermophile, DSM 2661, |
| | | helicase. | | | taxon: 2190 |
| Mja Hyp-1 | Theo. | Hypothetical protein- | | Methanococcus jannaschii | Thermophile, DSM 2661, |
| | | 1, ansR 5′-region | | | taxon: 2190 |
| Mja IF2 | Theo. | Translation initiation | Mja IF-2 | Methanococcus jannaschii | Thermophile, DSM 2661, |
| | | factor | | | taxon: 2190 |
| Mja KlbA | Exp. | KlbA, kilB operon | Mja KlbA | Methanococcus jannaschii | Thermophile, DSM 2661, |
| | | ORF A | | | taxon: 2190 |
| Mja PEP | Exp. | Phosphoenolpyruvate | | Methanococcus jannaschii | Thermophile, DSM 2661, |
| | | synthase | | | taxon: 2190 |
| Mja Pol-1 | Theo. | DNA polymerase | Pko Pol-1 | Methanococcus jannaschii | Thermophile, DSM 2661, |
| | | (alpha family) | | | taxon: 2190 |
| Mja Pol-2 | Theo. | DNA polymerase | Tli Pol-1 | Methanococcus jannaschii | Thermophile, DSM 2661, |
| | | (alpha family) | | | taxon: 2190 |
| Mja r-Gyr | Theo. | Reverse gyrase | Mja r-Gyr | Methanococcus jannaschii | Thermophile, DSM 2661, |
| | | | | | taxon: 2190 |
| Mja RFC-1 | Theo. | Replication factor C | Mja RFC-1 | Methanococcus jannaschii | Thermophile, DSM 2661, |
| | | | | | taxon: 2190 |
| Mja RFC-2 | Theo. | Replication factor C | Mja RFC-2 | Methanococcus jannaschii | Thermophile, DSM 2661, |
| | | | | | taxon: 2190 |
| Mja RFC-3 | Theo. | Replication factor C | Mja RFC-3 | Methanococcus jannaschii | Thermophile, DSM 2661, |
| | | | | | taxon: 2190 |
| Mja RNR-1 | Theo. | Anaerobic rNTP | | Methanococcus jannaschii | Thermophile, DSM 2661, |
| | | reductase | | | taxon: 2190 |
| Mja RNR-2 | Theo. | Anaerobic rNTP | | Methanococcus jannaschii | Thermophile, DSM 2661, |
| | | reductase | | | taxon: 2190 |
| Mja Rpol A″ | Theo. | RNA polymerase | | Methanococcus jannaschii | Thermophile, DSM 2661, |
| | | subunit A″ | | | taxon: 2190 |
| Mja Rpol A′ | Exp. | RNA polymerase | | Methanococcus jannaschii | Thermophile, DSM 2661, |
| | | subunit A′ | | | taxon: 2190 |
| Mja RtcB | Theo. | RNA terminal | Mja RtcB | Methanococcus jannaschii | Thermophile, DSM 2661, |
| (Mja Hyp-2) | | phosphate cyclase | | | taxon: 2190 |
| | | operon orfB | | | |
| Mja TFIIB | Theo. | Transcription factor | | Methanococcus jannaschii | Thermophile, DSM 2661, |
| | | IIB | | | taxon: 2190 |
| Mja UDP | Theo. | UDP-glucose | | Methanococcus jannaschii | Thermophile, DSM 2661, |
| GD | | dehydrogenase | | | taxon: 2190 |
| Mkn ATPase | Theo. | ATPase of the | | Methanopyrus kandleri | Thermophile, taxon: 190192 |
| | | AAA+ class, Cdc48 | | AV19 | |
| Mkn NtpB | Theo. | Archaeal/vacuolar- | | Methanopyrus kandleri | Thermophile, taxon: 190192 |
| | | type H+-ATPase | | AV19 | |
| | | subunit B | | | |
| Mkn RFC | Theo. | Replication factor C | Mja RFC-2 | Methanopyrus kandleri | Thermophile, taxon: 190192 |
| | | | | AV19 | |
| Mkn RtcB | Theo. | RNA terminal | Mja RtcB | Methanopyrus kandleri | Thermophile, taxon: 190192 |
| | | phosphate cyclase | | AV19 | |
| | | operon orfB | | | |
| Mth RIR1 | Exp. | Ribonucleoside- | Pfu RIR1-2 | Methanobacterium | Thermophile, delta H strain |
| | | diphosphate | | thermoautotrophicum | |
| | | reductase, alpha | | | |
| | | subunit | | | |
| Pab CDC21-1 | Theo. | Cell division control | Pho | Pyrococcus abyssi | Thermophile, strain Orsay, |
| | | protein 21 | CDC21-1 | | taxon: 29292 |
| Pab CDC21-2 | Theo. | Cell division control | Pho | Pyrococcus abyssi | Thermophile, strain Orsay, |
| | | protein 21 | CDC21-2 | | taxon: 29292 |
| Pab IF2 | Theo. | Translation initiation | Mja IF-2 | Pyrococcus abyssi | Thermophile, strain Orsay, |
| | | factor | | | taxon: 29292 |
| Pab KlbA | Theo. | Klba, kilB operon | Mja KlbA | Pyrococcus abyssi | Thermophile, strain Orsay, |
| | | ORF A | | | taxon: 29292 |
| Pab Lon | Theo. | ATP dependent | Pho Lon | Pyrococcus abyssi | Thermophile, strain Orsay, |
| | | protease LA | | | taxon: 29292 |
| Pab Moaa | Theo. | Molybdenum | | Pyrococcus abyssi | Thermophile, strain Orsay, |
| | | cofactor biosynthesis | | | taxon: 29292 |
| | | homolog | | | |
| Pab Pol II | Theo. | DNA polymerase II, | Pho Pol II | Pyrococcus abyssi | Thermophile, strain Orsay, |
| | | DP2 subunit | | | taxon: 29292 |
| Pab RFC-1 | Theo. | Replication factor C | Mja RFC-1 | Pyrococcus abyssi | Thermophile, strain Orsay, |
| | | | | | taxon: 29292 |
| Pab RFC-2 | Theo. | Replication factor C | Mja RFC-3 | Pyrococcus abyssi | Thermophile, strain Orsay, |
| | | | | | taxon: 29292 |
| Pab RIR1-1 | Theo. | Ribonucleoside- | Pfu RIR1-1 | Pyrococcus abyssi | Thermophile, strain Orsay, |
| | | diphosphate | | | taxon: 29292 |
| | | reductase, alpha | | | |
| | | subunit | | | |
| Pab RIR1-2 | Theo. | Ribonucleoside | | Pyrococcus abyssi | Thermophile, strain Orsay, |
| | | diphosphate | | | taxon: 29292 |
| | | reductase | | | |
| Pab RIR1-3 | Theo. | Ribonucleoside- | Pfu RIR1-2 | Pyrococcus abyssi | Thermophile, strain Orsay, |
| | | diphosphate | | | taxon: 29292 |
| | | reductase, alpha | | | |
| | | subunit | | | |
| Pab RtcB | Theo. | RNA terminal | Mja RtcB | Pyrococcus abyssi | Thermophile, strain Orsay, |
| (Pab Hyp-2) | | phosphate cyclase | | | taxon: 29292 |
| | | operon orfB | | | |
| Pab VMA | Theo. | Vacuolar ATPase | Pho VMA | Pyrococcus abyssi | Thermophile, strain Orsay, |
| | | (H+-transporting | | | taxon: 29292 |
| | | ATP synthase), | | | |
| | | subunit A | | | |
| Pfu CDC21 | Theo. | Cell division control | Pho | Pyrococcus furiosus | Thermophile, taxon: 186497, |
| | | protein 21 | CDC21-2 | | DSM3638 |
| Pfu IF2 | Theo. | Translation initiation | Mja IF-2 | Pyrococcus furiosus | Thermophile, taxon: 186497, |
| | | factor | | | DSM3638 |
| Pfu KlbA | Theo. | KlbA, kilB operon | Mja KlbA | Pyrococcus furiosus | Thermophile, taxon: 186497, |
| | | ORF A | | | DSM3638 |
| Pfu Lon | Theo. | ATP-dependent | Pho Lon | Pyrococcus furiosus | Thermophile, taxon: 186497, |
| | | protease LA | | | DSM3638 |
| Pfu RFC | Theo. | Replication factor C | Mja RFC-1 | Pyrococcus furiosus | Thermophile, DSM3638, |
| | | | | | taxon: 186497 |
| Pfu RIR1-1 | Exp. | Ribonucleoside- | Pfu RIR1-1 | Pyrococcus furiosus | Thermophile, taxon: 186497, |
| | | diphosphate | | | DSM3638 |
| | | reductase, alpha | | | |
| | | subunit | | | |
| Pfu RIR1-2 | Exp. | Ribonucleoside- | Pfu RIR1-2 | Pyrococcus furiosus | Thermophile, taxon: 186497, |
| | | diphosphate | | | DSM3638 |
| | | reductase, alpha | | | |
| | | subunit | | | |
| Pfu RtcB | Theo. | RNA terminal | Mja RtcB | Pyrococcus furiosus | Thermophile, taxon: 186497, |
| (Pfu Hyp-2) | | phosphate cyclase | | | DSM3638 |
| | | operon orfB | | | |
| Pfu TopA | Theo. | DNA topoisomerase I | Mja r-Gyr | Pyrococcus furiosus | Thermophile, taxon: 186497, |
| | | | | | DSM3638 |
| Pfu VMA | Theo. | Vacuolar ATPase | Pho VMA | Pyrococcus furiosus | Thermophile, taxon: 186497, |
| | | (H+-transporting | | | DSM3638 |
| | | ATP synthase), | | | |
| | | subunit A | | | |
| Pho CDC21-1 | Theo. | Cell division control | Pho | Pyrococcus horikoshii | Thermophile, taxon: 53953 |
| | | protein 21 | CDC21-1 | OT3 | |
| Pho CDC21-2 | Theo. | Cell division control | Pho | Pyrococcus horikoshii | Thermophile, taxon: 53953 |
| | | protein 21 | CDC21-2 | OT3 | |
| Pho IF2 | Theo. | Translation initiation | Mja IF-2 | Pyrococcus horikoshii | Thermophile, taxon: 53953 |
| | | factor | | OT3 | |
| Pho KlbA | Theo. | KlbA, kilB operon | Mja KlbA | Pyrococcus horikoshii | Thermophile, taxon: 53953 |
| | | ORF A | | OT3 | |
| Pho LHR | Theo. | Large helicase | | Pyrococcus horikoshii | Thermophile, taxon: 53953 |
| | | related protein | | OT3 | |
| Pho Lon | Theo. | ATP-dependent | Pho Lon | Pyrococcus horikoshii | Thermophile, taxon: 53953 |
| | | protease LA | | OT3 | |
| Pho Pol I | Theo. | DNA polymerase | Tli Pol-1 | Pyrococcus horikoshii | Thermophile, taxon: 53953 |
| | | (alpha family) | | OT3 | |
| Pho Pol II | Theo. | DNA polymerase II, | Pho Pol II | Pyrococcus horikoshii | Thermophile, taxon: 53953 |
| | | DP2 subunit | | OT3 | |
| Pho r-Gyr | Theo. | Reverse gyrase | Mja r-Gyr | Pyrococcus horikoshii | Thermophile, taxon: 53953 |
| | | | | OT3 | |
| Pho RadA | Theo. | RadA DNA repair | | Pyrococcus horikoshii | Thermophile, taxon: 53953 |
| | | protein | | OT3 | |
| Pho RFC | Theo. | Replication factor C | Mja RFC-1 | Pyrococcus horikoshii | Thermophile, taxon: 53953 |
| | | | | OT3 | |
| Pho RIR1 | Theo. | Ribonucleoside- | Pfu RIR1-2 | Pyrococcus horikoshii | Thermophile, taxon: 53953 |
| | | diphosphate | | OT3 | |
| | | reductase, alpha | | | |
| | | subunit | | | |
| Pho RtcB | Theo. | RNA terminal | Mja RtcB | Pyrococcus horikoshii | Thermophile, taxon: 53953 |
| (Pho Hyp-2) | | phosphate cyclase | | OT3 | |
| | | operon orfB | | | |
| Pho VMA | Theo. | Vacuolar ATPase | Pho VMA | Pyrococcus horikoshii | Thermophile, taxon: 53953 |
| | | (H+-transporting | | OT3 | |
| | | ATP synthase), | | | |
| | | subunit A | | | |
| Psp-GBD | Exp. | DNA polymerase | Tli Pol-1 | Pyrococcus spp. GB-D | Thermophile |
| Pol | | (alpha family) | | | |
| Tac- | Exp. | Vacuolar ATPase | Pho VMA | Thermoplasma | Thermophile, taxon: 2303 |
| ATCC25905 | | (H+-transporting | | acidophilum, ATCC | |
| VMA | | ATP synthase), | | 25905 | |
| | | subunit A | | | |
| Tac- | Theo. | Vacuolar ATPase | Pho VMA | Thermoplasma | Thermophile, taxon: 2303 |
| DSM1728 | | (H+-transporting | | acidophilum, DSM1728 | |
| VMA | | ATP synthase), | | | |
| | | subunit A | | | |
| Tag Pol-1 | Exp. | DNA polymerase | Pko Pol-1 | Thermococcus aggregans | Thermophile, taxon: 110163 |
| (Tsp-TY | | (alpha family) | | | |
| Pol-1) | | | | | |
| Tag Pol-2 | Exp. | DNA polymerase | Tli Pol-1 | Thermococcus aggregans | Thermophile, taxon: 110163 |
| (Tsp-TY | | (alpha family) | | | |
| Pol-2) | | | | | |
| Tag Pol-3 | Exp. | DNA polymerase | Tli Pol-2 | Thermococcus aggregans | Thermophile, taxon: 110163 |
| (Tsp-TY | | (alpha family) | | | |
| Pol-3) | | | | | |
| Tfu Pol-1 | Exp. | DNA polymerase | Pko Pol-1 | Thermococcus fumicolans | Thermophilem, taxon: 46540 |
| | | (alpha family) | | | |
| Tfu Pol-2 | Exp. | DNA polymerase | Tli Pol-2 | Thermococcus fumicolans | Thermophile, taxon: 46540 |
| | | (alpha family) | | | |
| Thy Pol-1 | Exp. | DNA polymerase | Tli Pol-1 | Thermococcus | Thermophile, taxon: 46539 |
| | | (alpha family) | | hydrothermalis | |
| Thy Pol-2 | Exp. | DNA polymerase | Tli Pol-2 | Thermococcus | Thermophile, taxon: 46539 |
| | | (alpha family) | | hydrothermalis | |
| Tko Pol-1 | Exp. | DNA polymerase | Pko Pol-1 | Pyrococcus/Thermococcus | Thermophile, taxon: 69014 |
| (Pko Pol-1) | | (alpha family) | | kodakaraensis KOD1 | |
| Tko Pol-2 | Exp. | DNA polymerase | Tli Pol-1 | Pyrococcus/Thermococcus | Thermophile, taxon: 69014 |
| (Pko Pol-2) | | (alpha family) | | kodakaraensis KOD1 | |
| Tli Pol-1 | Exp. | DNA polymerase | Tli Pol-1 | Thermococcus litoralis | Thermophile, taxon: 2265 |
| | | (alpha family) | | | |
| Tli Pol-2 | Exp. | DNA polymerase | Tli Pol-2 | Thermococcus litoralis | Thermophile, taxon: 2265 |
| | | (alpha family) | | | |
| Tsp-GE8 | Exp. | DNA polymerase | Tli Pol-1 | Thermococcus sp. GE8 | Thermophile, taxon: 105583 |
| Pol-1 | | (alpha family) | | | |
| Tsp-GE8 | Exp. | DNA polymerase | Tli Pol-2 | Thermococcus sp. GE8 | Thermophile, taxon: 105583 |
| Pol-2 | | (alpha family) | | | |
| Tvo VMA | Theo. | Vacuolar ATPase | Pho VMA | Thermoplasma volcanium | Thermophile, taxon: 50339 |
| | | (H+-transporting | | GSS1 | |
| | | ATP synthase), | | | |
| | | subunit A | | | |

What is claimed is:

1. An isolated nucleic acid molecule comprising a nucleotide sequence encoding at least one modified intein, a functional derivative or homolog thereof, wherein said nucleotide sequence further comprises one or more recombination sites.

2. An isolated nucleic acid molecule comprising: (a) a nucleotide sequence encoding at least one modified intein, a functional derivative or homolog thereof, wherein said nucleotide sequence further comprises one or more recombination sites; and (b) one or more topoisomerase recognition sites and/or one or more topoisomerases.

3. An isolated nucleic acid molecule comprising a nucleotide sequence encoding at least one modified intein, a functional derivative or homolog thereof, wherein said nucleotide sequence further comprises one or more recombination sites.

4. An isolated nucleic acid molecule comprising: (a) a nucleotide sequence encoding at least one modified intein, a functional derivative or homolog thereof, wherein said nucleotide sequence further comprises one or more recombination sites; and (b) one or more topoisomerase recognition sites and/or one or more topoisomerases.

5. The nucleic acid molecule of claim 1 or 3, wherein said nucleic acid molecule is a linear or circular molecule.

6. The nucleic acid molecule of claim 1 or 3, wherein said nucleic acid molecule comprises two or more recombination sites.

7. The nucleic acid molecule of claim 1, 2. 3, or 4, wherein one recombination site is flanked on one end by a cloning site.

8. The nucleic acid molecule of claim 7, wherein said cloning site contains one or more one unique restriction enzyme sites

9. The nucleic acid molecule of claim 6, wherein at least one of said two or more recombination sites flanks one of the ends of a topoisomerase recognition site in said molecule.

10. The nucleic acid molecule of claim 1 or 3, wherein said recombination sites are selected from the group consisting of:
(a) attB sites,
(b) attP sites,
(c) attL sites,
(d) attR sites,
(e) lox sites,
(f) psi sites,
(g) dif sites,
(h) cer sites,
(i) frt sites,
and mutants, variants, and derivatives of the recombination sites of (a), (b), (c), (d), (e), (f), (g), (h) or (i) which retain the ability to undergo recombination.

11. The nucleic acid molecule of claim 1 or 3, wherein said topoisomerase recognition site is recognized and bound by a type I topoisomerase.

12. The nucleic acid molecule of claim 11, wherein said type I topoisomerase is a type IB topoisomerase.

13. The nucleic acid molecule of claim 12, wherein said type IB topoisomerase is selected from the group consisting of eukaryotic nuclear type I topoisomerase and a poxvirus topoisomerase.

14. The nucleic acid molecule of claim 13, wherein said poxvirus topoisomerase is produced by or isolated from a virus selected from the group consisting of vaccinia virus, Shope fibroma virus, ORF virus, fowlpox virus, molluscum contagiosum virus and Amsacta moorei entomopoxvirus.

15. The nucleic acid molecule of claim 1 or 3, wherein said nucleic acid molecule further comprises a nucleotide sequence encoding a selectable marker.

16. The nucleic acid molecule of claim 1 or 3, wherein said nucleic acid molecule further contains a prokaryiotic or eukaryotic origin of replication.

17. The nucleic acid molecule of claim 1, wherein the nucleotide sequence encoding the at least one modified intein is operably linked to a sequence which is capable of regulating transcription.

18. The nucleic acid molecule of claim 17, wherein the sequence which is capable of regulating transcription is selected from the group consisting of:
(a) a promoter;
(b) an enhancer; and
(c) a repressor.

19. The nucleic acid molecule of claim 18, wherein the promoter is either an inducible promoter or a constitutive promoter.

20. A vector comprising the nucleic acid molecule of claim 1 or 3.

21. The vector of claim 20, wherein said vector is an expression vector.

22. The expression vector of claim 21, wherein the expression vector is selected from the group consisting of pcDNAGW-DT(sc), Pentr-DT(sc), pcDNA-DEST41, Pentr/D-TOPO, Pentr/SD/D-TOPO, pcDNA3.2/V5/GWD-TOPO and pcDNA6.2/V5/GWD-TOPO.

23. A host cell comprising the isolated nucleic acid molecule of claim 1.

24. A host cell comprising the vector of claim 21.

25. A host cell comprising the vector of claim 22.

26. A method of cloning at least one hybrid nucleic acid molecule comprising:
(a) providing at least a first population of nucleic acid molecules comprising at least one modified intein nucleotide sequence or a functional derivative or homolog thereof wherein all or a portion of such molecules contain at least a first and a second recombination site;
(b) providing at least a second population of nucleic acid molecules encoding at least one negative selectable marker located between a third and a fourth recombination site, and wherein either the first or the second recombination site of the first population of nucleic acid molecules is capable of recombining with either the third or the fourth recombination site of the second population of nucleic acid molecules;
(c) conducting a recombination reaction such that all or a portion of the molecules in the first population comprising at least one modified intein nucleotide sequence or a functional derivative or homolog thereof is recombined with one or more molecules from the second population to form a third population of hybrid nucleic acid molecules;
(d) cloning the third population of hybrid nucleic acid molecules; and
(e) introducing the cloned hybrid nucleic acid molecules into a suitable host cell.

27. A method of cloning at least one hybrid nucleic acid molecule comprising:
(a) providing a first population of first nucleic acid segments comprising at least one modified intein nucleotide sequence or a functional derivative or homolog thereof, wherein the first nucleic acid segment contains a first and a second recombination site;
(b) providing a second population of nucleic acid molecules comprising a vector molecule containing a nucleic acid molecule encoding at least one negative selectable marker, wherein the selectable marker is located between a third and a fourth recombination site, wherein either the first or the second recombination site of the first nucleic acid segment is capable of recombining with either the third or the fourth recombination site of the second population of nucleic acid molecules;
(c) conducting a recombination reaction such that all or a portion of the first nucleic acid segments comprising at least one modified mutant intein nucleotide sequence or a functional derivative or homolog thereof are recombined with all or a portion of the vector molecules comprising at least one negative selectable marker to form a third population of hybrid nucleic acid molecules;
(d) cloning the third population of hybrid nucleic acid molecules; and
(e) introducing the cloned hybrid nucleic acid molecules into a suitable host cell.

28. The method of claim 26 or 27, wherein the recombination sites comprise one or more recombination sites selected from the group consisting of:
(a) lox sites;
(b) psi sites;
(c) dif sites;
(d) cer sites;
(e) frt sites;
(f) att sites; and
(g) mutants, variants, and derivatives of the recombination sites of (a), (b), (c), (d), (e), or (f) which retain the ability to undergo recombination.

29. The method of claim 26 or 27, wherein the recombination is caused by mixing the first population of nucleic acid molecules and the second population of nucleic acid molecules with one or more recombination proteins under conditions which favor the recombination.

30. The method of claim 29, wherein the recombination proteins comprise one or more proteins selected from the group consisting of:
(a) Cre;
(b) Int;
(c) IHF;
(d) Xis;
(e) Fis;
(f) Hin;
(g) Gin;
(h) Cin;
(i) Tn3 resolvase;
(j) TndX;
(k) XerC; and
(l) XerD.

31. The method of claim 26 or 27, further comprising selecting for the population of hybrid nucleic acid molecules.

32. The method of claim 31, further comprising selecting for the population of hybrid nucleic acid molecules and against the first population of nucleic acid molecules.

33. The method of claim 32, further comprising selecting against cointegrate molecules and byproduct molecules such that insertion of the first nucleic acid molecules and resolving of the resultant cointegrate results in excision or loss of the negative selectable marker and retention of the at least one intein nucleotide sequence or a functional derivative or homolog thereof.

34. The method of claim 26 or 27, wherein at least one of the nucleic acid molecules is operably linked to a sequence which is capable of regulating transcription.

35. The method of claim 34, wherein the sequence which is capable of regulating transcription is selected from the group consisting of:
(a) a promoter;
(b) an enhancer; and
(c) a repressor.

36. The method of claim 35, wherein the promoter is either an inducible promoter or a constitutive promoter.

37. The method of claim 26 or 27, wherein one or more of the nucleic acid molecules further encodes a selectable marker.

38. The method of claim 26 or 27, wherein one or more of the nucleic acid molecules contains a prokaryotic or eukaryotic origin of replication.

39. The method of claim 26 or 27, wherein translation of an RNA produced from the cloned nucleic acid molecules results in the production of a fusion protein.

40. The method of claim 26 or 27, wherein the second population of nucleic acid molecules further encodes a fusion protein.

41. The method of claim 26 or 27, wherein the second population of nucleic acid molecules further encodes a polypeptide selected from the group consisting of:
(a) the Fc portion of an immunoglobin;
(b) β-glucuronidase;
(c) a fluorescent protein;
(d) a purification tag; and
(e) an epitope tag.

42. The method of claim 41, wherein the second population of nucleic acid molecules further encodes a fluorescent protein selected from the group consisting of:
(a) green fluorescent protein;
(b) yellow fluorescent protein;
(c) red fluorescent protein; and
(d) cyan fluorescent protein.

43. The method of claim 41, wherein the second population of nucleic acid molecules further encodes an additional purification tag selected from the group consisting of:
(a) an epitope tag;
(b) maltose binding protein;
(c) a six histidine tag; and
(d) glutathione S-transferase.

44. The method of claim 26 or 27, wherein at least one of the nucleic acid molecules encodes all of part of an open reading frame and at least one of the nucleic acid molecules contains a sequence which is capable of regulating transcription.

45. The method of claim 26 or 27, wherein the nucleic acid molecules comprise nucleic acid molecules of one or more libraries.

46. The method of claim 45, wherein the one or more libraries comprise either Cdna or genomic DNA.

47. The method of claim 46, wherein the one or more libraries comprise nucleic acid molecules which encode variable domains of antibody molecules.

48. The method of claim 47, wherein the one or more libraries comprise nucleic acid molecules which encode variable domains of antibody light and heavy chains.

49. A composition comprising the population of hybrid nucleic acid molecules produced by the method of claim 26 or 27.

50. A population of recombinant host cells comprising the population of hybrid nucleic acid molecules produced by the method of claim 26 or 27.

51. A kit for cloning, joining, deleting, or replacing nucleic acid segments, the kit comprising (1) one or more recombination proteins or a composition comprising one or more recombination proteins, (2) at least one nucleic acid molecule comprising one or more recombination sites having at least two different recombination specificities, and (3) one or more components selected from the group consisting of:
(a) nucleic acid molecules comprising additional recombination sites;
(b) one or more enzymes having ligase activity;
(c) one or more enzymes having polymerase activity;
(d) one or more enzymes having reverse transcriptase activity;
(e) one or more enzymes having restriction endonuclease activity;
(f) one or more primers;
(g) one or more nucleic acid libraries;
(h) one or more supports;
(i) one or more buffers;
(j) one or more detergents or solutions containing detergents;
(k) one or more nucleotides;
(l) one or more terminating agents;
(m) one or more transfection reagents;
(n) one or more host cells; and
(o) instructions for using the kit components.

52. The kit of claim 51, wherein the recombination sites having at least three different recombination specificities each comprising att sites with different seven base pair overlap regions.

53. The kit of claim 51, wherein the composition comprising one or more recombination proteins is capable of catalyzing recombination between att sites.

54. The kit of claim 51, wherein the composition comprising one or more recombination proteins capable of catalyzing a BP reaction, an LR reaction, or both BP and LR reactions.
